SEQUENCE LISTING (78 sequences)

SEQ ID NO: 1 - protein, 20 aa, Unknown
  source: /note=“Description of Unknown Pf bacteriophage peptide”
MAAVPAEATSALTEAKADGV

SEQ ID NO: 2 - protein, 20 aa, Unknown
  source: /note=“Description of Unknown Pf bacteriophage peptide”
MAEVPAAATTALTDAQKDGV

SEQ ID NO: 3 - protein, 19 aa, Unknown
  source: /note=“Description of Unknown Pf bacteriophage peptide”
GVIDTSAVESAITDGQGDM

SEQ ID NO: 4 - protein, 19 aa, Unknown
  source: /note=“Description of Unknown Pf bacteriophage peptide”
GVIDTSAVEAAITEGKGDM

SEQ ID NO: 5 - protein, 19 aa, Unknown
  source: /note=“Description of Unknown Pf bacteriophage peptide”
GVIGTSAVESAITDGQGDM

SEQ ID NO: 6 - protein, 19 aa, Unknown
  source: /note=“Description of Unknown Pf bacteriophage peptide”
GVIDTSAVEAAITDGQGDM

SEQ ID NO: 7 - protein, 19 aa, Unknown
  source: /note=“Description of Unknown Pf bacteriophage peptide”
AVIDTSAVESAITDGQGDM

SEQ ID NO: 8 - protein, 19 aa, Unknown
  source: /note=“Description of Unknown Pf bacteriophage peptide”
SVIDTSSVEQAITDGKSDM

SEQ ID NO: 9 - protein, 17 aa, Unknown
  source: /note=“Description of Unknown Pf bacteriophage peptide”
GVIGAVALALAVGDGDM

SEQ ID NO: 10 - protein, 14 aa, Unknown
  source: /note=“Description of Unknown Pf bacteriophage peptide”
MAAEGDIDTTKALA

SEQ ID NO: 11 - protein, 20 aa, Unknown
  source: /note=“Description of Unknown Pf bacteriophage peptide”
AGWDYSSLTSDVDFSTIATG

SEQ ID NO: 12 - protein, 20 aa, Unknown
  source: /note=“Description of Unknown Pf bacteriophage peptide”
AGWDYSSLTSDVDFSTIATS

SEQ ID NO: 13 - protein, 20 aa, Unknown
  source: /note=“Description of Unknown Pf bacteriophage peptide”
AGWDYSSMTSDVDFSTIATG

SEQ ID NO: 14 - protein, 20 aa, Unknown
  source: /note=“Description of Unknown Pf bacteriophage peptide”
AGWDYSTLTSGIDFGTIATG

SEQ ID NO: 15 - protein, 20 aa, Unknown
  source: /note=“Description of Unknown Pf bacteriophage peptide”
AGWDYSVLTSDVDFGTIATG

SEQ ID NO: 16 - protein, 20 aa, Unknown
  source: /note=“Description of Unknown Pf bacteriophage peptide”
AGWDYSSLTSDVDYSTVATG

SEQ ID NO: 17 - protein, 20 aa, Unknown
  source: /note=“Description of Unknown Pf bacteriophage peptide”
AGWDYSGLTSDIDFSTIATG

SEQ ID NO: 18 - protein, 20 aa, Unknown
  source: /note=“Description of Unknown Pf bacteriophage peptide”
AGWDYSGMTKDVDFSTIATG

SEQ ID NO: 19 - protein, 20 aa, Unknown
  source: /note=“Description of Unknown Pf bacteriophage peptide”
AGWDYSGLTGDIDFSTIATG

SEQ ID NO: 20 - protein, 21 aa, Unknown
  source: /note=“Description of Unknown Pf bacteriophage peptide”
FADSLIDETTKEVLTQAGTDG

SEQ ID NO: 21 - protein, 21 aa, Unknown
  source: /note=“Description of Unknown Pf bacteriophage peptide”
FAESLLDETTKGVLAQASTDG

SEQ ID NO: 22 - protein, 19 aa, Unknown
  source: /note=“Description of Unknown Pf bacteriophage peptide”
ESLLDETTKGVLAQASTDG

SEQ ID NO: 23 - protein, 21 aa, Unknown
  source: /note=“Description of Unknown Pf bacteriophage peptide”
FAESLLDETTKGALAQASTDG

SEQ ID NO: 24 - protein, 21 aa, Unknown
  source: /note=“Description of Unknown Pf bacteriophage peptide”
FAESLIDETTKEVLTQAGTDG

SEQ ID NO: 25 - protein, 21 aa, Unknown
  source: /note=“Description of Unknown Pf bacteriophage peptide”
FAESLLDETTKEVLTQAGTDG

SEQ ID NO: 26 - protein, 19 aa, Unknown
  source: /note=“Description of Unknown Pf bacteriophage peptide”
MADIFGAVDFSSVSTWVVS

SEQ ID NO: 27 - protein, 19 aa, Unknown
  source: /note=“Description of Unknown Pf bacteriophage peptide”
MAEVPAAATTALADAQKDG

SEQ ID NO: 28 - protein, 19 aa, Unknown
  source: /note=“Description of Unknown Pf bacteriophage peptide”
MAEVPAAATTALTDAQKDG

SEQ ID NO: 29 - protein, 19 aa, Unknown
  source: /note=“Description of Unknown Pf bacteriophage peptide”
MAEVPAAATTALTDAQRDG

SEQ ID NO: 30 - protein, 19 aa, Unknown
  source: /note=“Description of Unknown Pf bacteriophage peptide”
MAEVPADATAALADAKADG

SEQ ID NO: 31 - protein, 19 aa, Unknown
  source: /note=“Description of Unknown Pf bacteriophage peptide”
MAAVPAEATSALTEAKADG

SEQ ID NO: 32 - protein, 19 aa, Unknown
  source: /note=“Description of Unknown Pf bacteriophage peptide”
AEGDLDFSSMISSVNGSAV

SEQ ID NO: 33 - protein, 19 aa, Unknown
  source: /note=“Description of Unknown Pf bacteriophage peptide”
SAAVPEEATKALTTAGTDV

SEQ ID NO: 34 - protein, 15 aa, Artificial Sequence
  source: /note=“Description of Artificial Sequence Synthetic peptide”
  MOD_RES (7)..(7): a neutral amino acid
  MOD_RES (11)..(11): an acidic amino acid
  MOD_RES (13)..(13): a neutral or basic amino acid
DTSAVEXAITXGXGD

SEQ ID NO: 35 - protein, 18 aa, Artificial Sequence
  source: /note=“Description of Artificial Sequence Synthetic peptide”
  MOD_RES (1)..(1): an acidic amino acid
  MOD_RES (4)..(4): a neutral amino acid
  MOD_RES (10)..(10): a neutral or acidic amino acid
  MOD_RES (13)..(13): a neutral amino acid
  MOD_RES (16)..(16): a neutral amino acid
XSLXDETTKXVLXQAXTD

SEQ ID NO: 36 - protein, 20 aa, Artificial Sequence
  source: /note=“Description of Artificial Sequence Synthetic peptide”
GVIDTSAVESAITDGQGDMC

SEQ ID NO: 37 - protein, 20 aa, Artificial Sequence
  source: /note=“Description of Artificial Sequence Synthetic peptide”
ESLLDETTKGVLAQASTDGC

SEQ ID NO: 38 - protein, 20 aa, Artificial Sequence
  source: /note=“Description of Artificial Sequence Synthetic peptide”
GVIDTSAVEAAITEGKGDMC

SEQ ID NO: 39 - protein, 20 aa, Unknown
  source: /note=“Description of Unknown Pf bacteriophage peptide”
GSVIDTSAVESAITDGQGDM

SEQ ID NO: 40 - protein, 19 aa, Unknown
  source: /note=“Description of Unknown Pf bacteriophage peptide”
GVIDTSAVESAITEGQGDM

SEQ ID NO: 41 - protein, 20 aa, Unknown
  source: /note=“Description of Unknown Pf bacteriophage peptide”
GSVIDTSAVEAAITEGKGDM

SEQ ID NO: 42 - protein, 19 aa, Unknown
  source: /note=“Description of Unknown Pf bacteriophage peptide”
GSVIDTSAVESAITDGQGD

SEQ ID NO: 43 - protein, 18 aa, Unknown
  source: /note=“Description of Unknown Pf bacteriophage peptide”
GVIDTSAVESAITDGQGD

SEQ ID NO: 44 - protein, 19 aa, Unknown
  source: /note=“Description of Unknown Pf bacteriophage peptide”
GSVIDTSAVEAAITEGKGD

SEQ ID NO: 45 - protein, 20 aa, Unknown
  source: /note=“Description of Unknown Pf bacteriophage peptide”
AESLLDETTKEVLTQAGTDG

SEQ ID NO: 46 - protein, 20 aa, Unknown
  source: /note=“Description of Unknown Pf bacteriophage peptide”
ADSLIDETTKEVLTQAGTDG

SEQ ID NO: 47 - protein, 20 aa, Unknown
  source: /note=“Description of Unknown Pf bacteriophage peptide”
AESLIDETTKGVLAQASTDG

SEQ ID NO: 48 - protein, 19 aa, Unknown
  source: /note=“Description of Unknown Pf bacteriophage peptide”
DSLIDETTKEVLTQAGTDG

SEQ ID NO: 49 - protein, 20 aa, Unknown
  source: /note=“Description of Unknown Pf bacteriophage peptide”
AESLLDETTKGVLAQASTDG

SEQ ID NO: 50 - protein, 17 aa, Unknown
  source: /note=“Description of Unknown Pf bacteriophage peptide”
DYSSLTSDVDFSTIATG

SEQ ID NO: 51 - protein, 19 aa, Unknown
  source: /note=“Description of Unknown Pf bacteriophage peptide”
GWDYSSLTSDVDFSTIATG

SEQ ID NO: 52 - protein, 19 aa, Unknown
  source: /note=“Description of Unknown Pf bacteriophage peptide”
AEGDIDTTKALAYIAGGLT

SEQ ID NO: 53 - protein, 20 aa, Unknown
  source: /note=“Description of Unknown Pf bacteriophage peptide”
CESLLDETTKGVLAQASTDG

SEQ ID NO: 54 - protein, 19 aa, Unknown
  source: /note=“Description of Unknown Pf bacteriophage peptide”
ESLLDETTKCVLAQASTDG

SEQ ID NO: 55 - protein, 20 aa, Artificial Sequence
  source: /note=“Description of Artificial Sequence Synthetic peptide”
DSLIDETTKEVLTQAGTDGK

SEQ ID NO: 56 - protein, 20 aa, Artificial Sequence
  source: /note=“Description of Artificial Sequence Synthetic peptide”
GVIDTSAVESAITDGQGDMK

SEQ ID NO: 57 - protein, 17 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (9)..(9): a neutral amino acid
  MOD_RES (13)..(13): an acidic amino acid
  MOD_RES (15)..(15): a neutral or basic amino acid
XXDTSAVEXAITXGXGD

SEQ ID NO: 58 - protein, 18 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (9)..(9): a neutral amino acid
  MOD_RES (13)..(13): an acidic amino acid
  MOD_RES (15)..(15): a neutral or basic amino acid
XXDTSAVEXAITXGXGDM

SEQ ID NO: 59 - protein, 18 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (3)..(3): a neutral amino acid
  MOD_RES (10)..(10): a neutral amino acid
  MOD_RES (14)..(14): an acidic amino acid
  MOD_RES (16)..(16): a neutral or basic amino acid
XXXDTSAVEXAITXGXGD

SEQ ID NO: 60 - protein, 19 aa, Artificial Sequence
  source: /note=“Description of Artificial Sequence Synthetic peptide”
  MOD_RES (1)..(1): an acidic amino acid
  MOD_RES (4)..(4): a neutral amino acid
  MOD_RES (10)..(10): a neutral or acidic amino acid
  MOD_RES (13)..(13): a neutral amino acid
  MOD_RES (16)..(16): a neutral amino acid
XSLXDETTKXVLXQAXTDG

SEQ ID NO: 61 - protein, 20 aa, Artificial Sequence
  source: /note=“Description of Artificial Sequence Synthetic peptide”
  MOD_RES (2)..(2): an acidic amino acid
  MOD_RES (5)..(5): a neutral amino acid
  MOD_RES (11)..(11): a neutral or acidic amino acid
  MOD_RES (14)..(14): a neutral amino acid
  MOD_RES (17)..(17): a neutral amino acid
AXSLXDETTKXVLXQAXTDG

SEQ ID NO: 62 - protein, 20 aa, Unknown
  source: /note=“Description of Unknown Pf bacteriophage peptide”
DSLIDETTKEVLTQAGTDGC

SEQ ID NO: 63 - protein, 21 aa, Unknown
  source: /note=“Description of Unknown Pf bacteriophage peptide”
AESLLDETTKGVLAQASTDGC

SEQ ID NO: 64 - protein, 20 aa, Unknown
  source: /note=“Description of Unknown Pf bacteriophage peptide”
GWDYSSLTSDVDFSTIATGC

SEQ ID NO: 65 - protein, 20 aa, Unknown
  source: /note=“Description of Unknown Pf bacteriophage peptide”
AEGDIDTTKALAYIAGGLTC

SEQ ID NO: 66 - protein, 18 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (3)..(3): a neutral amino acid
  MOD_RES (4)..(4): an acidic or neutral amino acid
  MOD_RES (5)..(5): a neutral amino acid
  MOD_RES (6)..(6): a neutral amino acid
  MOD_RES (7)..(7): a neutral amino acid
  MOD_RES (8)..(8): a neutral amino acid
  MOD_RES (9)..(9): an acidic amino acid
  MOD_RES (10)..(10): a neutral amino acid
  MOD_RES (11)..(11): a neutral amino acid
  MOD_RES (12)..(12): a neutral amino acid
  MOD_RES (13)..(13): a neutral amino acid
  MOD_RES (14)..(14): an acidic amino acid
  MOD_RES (15)..(15): a neutral amino acid
  MOD_RES (16)..(16): a neutral or basic amino acid
  MOD_RES (17)..(17): a neutral amino acid
  MOD_RES (18)..(18): an acidic amino acid
XXXXXXXXXXXXXXXXXX

SEQ ID NO: 67 - protein, 19 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (3)..(3): a neutral amino acid
  MOD_RES (4)..(4): acidic or neutral amino acid
  MOD_RES (5)..(5): a neutral amino acid
  MOD_RES (6)..(6): a neutral amino acid
  MOD_RES (7)..(7): a neutral amino acid
  MOD_RES (8)..(8): a neutral amino acid
  MOD_RES (9)..(9): an acidic amino acid
  MOD_RES (10)..(10): a neutral amino acid
  MOD_RES (11)..(11): a neutral amino acid
  MOD_RES (12)..(12): a neutral amino acid
  MOD_RES (13)..(13): a neutral amino acid
  MOD_RES (14)..(14): an acidic amino acid
  MOD_RES (15)..(15): a neutral amino acid
  MOD_RES (16)..(16): a neutral or basic amino acid
  MOD_RES (17)..(17): a neutral amino acid
  MOD_RES (18)..(18): an acidic amino acid
  MOD_RES (19)..(19): a neutral amino acid
XXXXXXXXXXXXXXXXXXX

SEQ ID NO: 68 - protein, 19 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (3)..(3): a neutral amino acid
  MOD_RES (4)..(4): a neutral amino acid
  MOD_RES (5)..(5): acidic or neutral amino acid
  MOD_RES (6)..(6): a neutral amino acid
  MOD_RES (7)..(7): a neutral amino acid
  MOD_RES (8)..(8): a neutral amino acid
  MOD_RES (9)..(9): a neutral amino acid
  MOD_RES (10)..(10): an acidic amino acid
  MOD_RES (11)..(11): a neutral amino acid
  MOD_RES (12)..(12): a neutral amino acid
  MOD_RES (13)..(13): a neutral amino acid
  MOD_RES (14)..(14): a neutral amino acid
  MOD_RES (15)..(15): an acidic amino acid
  MOD_RES (16)..(16): a neutral amino acid
  MOD_RES (17)..(17): a neutral or basic amino acid
  MOD_RES (18)..(18): a neutral amino acid
  MOD_RES (19)..(19): an acidic amino acid
XXXXXXXXXXXXXXXXXXX

SEQ ID NO: 69 - protein, 20 aa, Artificial Sequence
  source: /note=“Description of Artificial Sequence Synthetic peptide”
  MOD_RES (1)..(1): a neutral amino acid
  MOD_RES (2)..(2): an acidic amino acid
  MOD_RES (3)..(3): a neutral amino acid
  MOD_RES (4)..(4): a neutral amino acid
  MOD_RES (5)..(5): a neutral amino acid
  MOD_RES (6)..(6): an acidic amino acid
  MOD_RES (7)..(7): an acidic amino acid
  MOD_RES (8)..(8): a neutral amino acid
  MOD_RES (9)..(9): a neutral amino acid
  MOD_RES (10)..(10): a basic amino acid
  MOD_RES (11)..(11): an acidic or neutral amino acid
  MOD_RES (12)..(12): a neutral amino acid
  MOD_RES (13)..(13): a neutral amino acid
  MOD_RES (14)..(14): a neutral amino acid
  MOD_RES (15)..(15): a neutral amino acid
  MOD_RES (16)..(16): a neutral amino acid
  MOD_RES (17)..(17): a neutral amino acid
  MOD_RES (18)..(18): a neutral amino acid
  MOD_RES (19)..(19): an acidic amino acid
  MOD_RES (20)..(20): a neutral amino acid
XXXXXXXXXXXXXXXXXXXX

SEQ ID NO: 70 - protein, 21 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (3)..(3): an acidic amino acid
  MOD_RES (4)..(4): a neutral amino acid
  MOD_RES (5)..(5): a neutral amino acid
  MOD_RES (6)..(6): a neutral amino acid
  MOD_RES (7)..(7): an acidic amino acid
  MOD_RES (8)..(8): an acidic amino acid
  MOD_RES (9)..(9): a neutral amino acid
  MOD_RES (10)..(10): a neutral amino acid
  MOD_RES (11)..(11): a basic amino acid
  MOD_RES (12)..(12): an acidic or neutral amino acid
  MOD_RES (13)..(13): a neutral amino acid
  MOD_RES (14)..(14): a neutral amino acid
  MOD_RES (15)..(15): a neutral amino acid
  MOD_RES (16)..(16): a neutral amino acid
  MOD_RES (17)..(17): a neutral amino acid
  MOD_RES (18)..(18): a neutral amino acid
  MOD_RES (19)..(19): a neutral amino acid
  MOD_RES (20)..(20): an acidic amino acid
  MOD_RES (21)..(21): a neutral amino acid
XXXXXXXXXXXXXXXXXXXXX

SEQ ID NO: 71 - protein, 14 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (3)..(3): a neutral amino acid
  MOD_RES (4)..(4): an acidic amino acid
  MOD_RES (5)..(5): a neutral amino acid
  MOD_RES (6)..(6): an acidic amino acid
  MOD_RES (7)..(7): a neutral amino acid
  MOD_RES (8)..(8): an acidic amino acid
  MOD_RES (9)..(9): a neutral amino acid
  MOD_RES (10)..(10): a neutral amino acid
  MOD_RES (11)..(11): a basic amino acid
  MOD_RES (12)..(12): a neutral amino acid
  MOD_RES (13)..(13): a neutral amino acid
  MOD_RES (14)..(14): a neutral amino acid
XXXXXXXXXXXXXX

SEQ ID NO: 72 - protein, 20 aa, Artificial Sequence
  source: /note=“Description of Artificial Sequence Synthetic peptide”
  MOD_RES (1)..(1): a neutral amino acid
  MOD_RES (2)..(2): a neutral amino acid
  MOD_RES (3)..(3): an acidic amino acid
  MOD_RES (4)..(4): a neutral amino acid
  MOD_RES (5)..(5): a neutral amino acid
  MOD_RES (6)..(6): an acidic or neutral amino acid
  MOD_RES (7)..(7): a neutral amino acid
  MOD_RES (8)..(8): a neutral amino acid
  MOD_RES (9)..(9): a neutral amino acid
  MOD_RES (10)..(10): a neutral amino acid
  MOD_RES (11)..(11): a neutral amino acid
  MOD_RES (12)..(12): a neutral amino acid
  MOD_RES (13)..(13): a neutral amino acid
  MOD_RES (14)..(14): an acidic amino acid
  MOD_RES (15)..(15): a neutral amino acid
  MOD_RES (16)..(16): a neutral or basic amino acid
  MOD_RES (17)..(17): a neutral or basic amino acid
  MOD_RES (18)..(18): an acidic amino acid
  MOD_RES (19)..(19): a neutral amino acid
  MOD_RES (20)..(20): a neutral amino acid
XXXXXXXXXXXXXXXXXXXX

SEQ ID NO: 73 - protein, 19 aa, Artificial Sequence
  source: /note=“Description of Artificial Sequence Synthetic peptide”
  MOD_RES (3)..(3): an acidic amino acid
  VARIANT (7)..(7): /replace=“an acidic amino acid”
  VARIANT (10)..(10): /replace=“T” or “S”
  VARIANT (13)..(13): /replace=“T”
  MOD_RES (14)..(14): an acidic amino acid
  VARIANT (16)..(16): /replace=“E”
  VARIANT (17)..(17): /replace=“A”
  MOD_RES (18)..(18): an acidic amino acid
  SITE (1)..(19): /note=“Variant residues given in the sequence have no preference with respect to those in the annotations for variant positions”
MAXVPAAATAALAXAKKXG

SEQ ID NO: 74 - protein, 19 aa, Artificial Sequence
  source: /note=“Description of Artificial Sequence Synthetic peptide”
  MOD_RES (3)..(3): an acidic amino acid
  MOD_RES (9)..(9): an acidic amino acid
MAXIFGAVXFSSVSTWVVS

SEQ ID NO: 75 - protein, 20 aa, Artificial Sequence
  source: /note=“Description of Artificial Sequence Synthetic peptide”
  MOD_RES (4)..(4): an acidic amino acid
  VARIANT (7)..(7): /replace=“G” or “T” or “V”
  VARIANT (8)..(8): /replace=“M”
  VARIANT (10)..(10): /replace=“K” or “G”
  MOD_RES (11)..(11): an acidic amino acid
  VARIANT (12)..(12): /replace=“I”
  MOD_RES (13)..(13): an acidic amino acid
  VARIANT (14)..(14): /replace=“Y”
  VARIANT (15)..(15): /replace=“G”
  VARIANT (17)..(17): /replace=“V”
  VARIANT (20)..(20): /replace=“S”
  SITE (1)..(20): /note=“Variant residues given in the sequence have no preference with respect to those in the annotations for variant positions”
AGWXYSSLTSXVXFSTIATG

SEQ ID NO: 76 - protein, 19 aa, Artificial Sequence
  source: /note=“Description of Artificial Sequence Synthetic peptide”
  MOD_RES (2)..(2): an acidic amino acid
  MOD_RES (4)..(4): an acidic amino acid
  MOD_RES (6)..(6): an acidic amino acid
AXGXLXFSSMISSVDGSAV

SEQ ID NO: 77 - protein, 18 aa, Artificial Sequence
  source: /note=“Description of Artificial Sequence Synthetic peptide”
  VARIANT (1)..(1): /replace=“A” or “S”
  MOD_RES (4)..(4): acidic or neutral amino acid
  VARIANT (7)..(7): /replace=“S”
  MOD_RES (9)..(9): an acidic amino acid
  VARIANT (10)..(10): /replace=“A” or “E”
  MOD_RES (14)..(14): an acidic amino acid
  VARIANT (16)..(16): /replace=“K”
  VARIANT (17)..(17): /replace=“S”
  MOD_RES (18)..(18): an acidic amino acid
  SITE (1)..(18): /note=“Variant residues given in the sequence have no preference with respect to those in the annotations for variant positions”
GVIXTSAVXSAITXGEGX

SEQ ID NO: 78 - protein, 20 aa, Artificial Sequence
  source: /note=“Description of Artificial Sequence Synthetic peptide”
  MOD_RES (2)..(2): an acidic amino acid
  VARIANT (5)..(5): /replace=“I”
  MOD_RES (6)..(6): an acidic amino acid
  MOD_RES (7)..(7): an acidic amino acid
  MOD_RES (11)..(11): acidic or neutral amino acid
  VARIANT (12)..(12): /replace=“A”
  VARIANT (14)..(14): /replace=“A”
  VARIANT (17)..(17): /replace=“S”
  MOD_RES (19)..(19): an acidic amino acid
  SITE (1)..(20): /note=“Variant residues given in the sequence have no preference with respect to those in the annotations for variant positions”
AXSLLXXTTKXVLTEAGTXG

(19) United States
(12) Patent Application Publication
(10) Pub. No.: US 2024/0325517 A1
(43) Pub. Date: Oct. 3, 2024

(54) COMPOSITIONS AND METHODS TARGETING FILAMENTOUS BACTERIOPHAGE

(71) Applicants: THE UNIVERSITY OF MONTANA, Missoula, MT (US); INIMMUNE CORPORATION, Missoula, MT (US)

(72) Inventors: Patrick Secor, Missoula, MT (US); Jay Evans, Corvallis, MT (US); David Burkhart, Missoula, MT (US)

(21) Appl. No.: 18/245,233

(22) PCT Filed: Sep. 15, 2021
(86) PCT No.: PCT/US2021/050551

§ 371 Date: Mar. 14, 2023

Related U.S. Application Data
(60) Provisional application No. 63/078,777, filed on Sep. 15, 2020.

(51) Int. Cl. A61K 39/12; A61K 39/00; A61P 31/04

(52) U.S. Cl. CPC A61K 39/12; A61P 31/04; A61K 2039/575; A61K 2039/6037; A61K 2039/627; A61K 2039/70; C07K 2319/55; C12N 2795/14234

(57) ABSTRACT

Multivalent conjugates and conjugate compositions of peptides derived from the CoaB coat protein of Pseudomonas aeruginosa filamentous (Pf) bacteriophage and conjugates adjuvanted with a TLR 4 and/or TLR 7/8 agonist induce and/or enhance an immune response toward Pseudomonas aeruginosa filamentous (Pf) bacteriophage. The conjugates, compositions, and vaccines have utility to reduce, prevent and/or ameliorate the adverse effect of Pseudomonas aeruginosa infection, such as biofilm formation.

### RELATED APPLICATIONS

This application claims the benefit of priority to U.S. provisional application Ser. No. 63/078,777, filed Sep. 15, 2020, which is incorporated herein by reference in its entirety.

### STATEMENT OF GOVERNMENT INTEREST

This invention was made with government support under grant number R01AI138981 awarded by The National Institutes of Health. The government has certain rights in the invention.

### TECHNICAL FIELD

The present invention relates to compositions and methods for inducing or enhancing an immune response toward a Pseudomonas aeruginosa filamentous (Pf) bacteriophage to reduce, prevent and/or ameliorate the adverse effect of Pseudomonas aeruginosa infection (e.g., biofilm formation).

### BACKGROUND

Pseudomonas aeruginosa is a gram-negative bacterial pathogen responsible for numerous acute and chronic infections in humans. Due to P. aeruginosa's dramatic economic and health impact on individuals and societies and because of the increasing antibiotic resistance of P. aeruginosa, the World Health Organization identifies it as one of the three antibiotic-resistant pathogens that pose the greatest threat to human health. Similarly, the United States Center for Disease Control has listed P. aeruginosa as one of six drug-resistance pathogens, which are primary drivers of increasing rates of Hospital Acquired Infections. In spite of decades of efforts there are no approved pharmaceutical therapies to treat or prevent P. aeruginosa (Pa). There is a long felt need for immunogenic compositions and vaccines to treat and prevent Pa infection.

### SUMMARY

In one aspect, the present invention provides an immunogenic conjugate comprising two or more different Pf bacteriophage peptides, or derivatives thereof, coupled to a carrier. The invention further provides an immunogenic composition comprising the immunogenic conjugate or more than one of the immunogenic conjugates that are different. The invention further provides a method of inducing or enhancing an immune response comprising administering to a subject in need thereof, an effective amount of the immunogenic conjugate, the immunogenic composition, or a vaccine thereof.

In another aspect, the invention provides an immunogenic conjugate comprising two or more different Pf bacteriophage peptides, or derivatives thereof, coupled to a carrier, or immunogenic compositions thereof, for use in inducing or enhancing an immune response.

In another aspect, the invention provides the use of an immunogenic conjugate comprising two or more different Pf bacteriophage peptides, or derivatives thereof, coupled to a carrier, or immunogenic compositions thereof, for the manufacture of a medicament for inducing or enhancing an immune response.

Another aspect of the present invention provides an immunogenic composition comprising: an immunogenic conjugate comprising a Pf bacteriophage peptide, or a derivative thereof, coupled to a carrier; and one or more adjuvants, the one or more adjuvants comprising a TLR4 agonist and/or a TLR7/8 agonist. The invention further provides a method of inducing or enhancing an immune response comprising administering to a subject in need thereof, an effective amount of the immunogenic composition, or a vaccine thereof.

Another aspect of the invention provides an immunogenic composition comprising: an immunogenic conjugate comprising a Pf bacteriophage peptide, or a derivative thereof, coupled to a carrier; and one or more adjuvants, the one or more adjuvants comprising a TLR4 agonist and/or a TLR7/8 agonist, for use in inducing or enhancing an immune response.

Another aspect of the invention provides the use of an immunogenic composition comprising: an immunogenic conjugate comprising a Pf bacteriophage peptide, or a derivative thereof, coupled to a carrier; and one or more adjuvants, the one or more adjuvants comprising a TLR4 agonist and/or a TLR7/8 agonist, in the manufacture of a medicament for inducing or enhancing an immune response.

In another aspect, the invention provides a method of inducing or enhancing an immune response comprising administering to a subject in need thereof, an effective amount of an immunogenic conjugate comprising a Pf bacteriophage peptide, or a derivative thereof, coupled to a carrier; and one or more adjuvants, the one or more adjuvants comprising a TLR4 agonist and/or a TLR7/8 agonist.

Another aspect of the invention provides a pharmaceutical combination of an immunogenic conjugate comprising a Pf bacteriophage peptide, or a derivative thereof, coupled to a carrier; and one or more adjuvants, the one or more adjuvants comprising a TLR4 agonist and/or a TLR7/8 agonist, for use in inducing or enhancing an immune response.

Another aspect of the invention provides use of a pharmaceutical combination of an immunogenic conjugate comprising a Pf bacteriophage peptide, or a derivative thereof, coupled to a carrier; and one or more adjuvants, the one or more adjuvants comprising a TLR4 agonist and/or a TLR7/8 agonist, in the manufacture of a medicament for inducing or enhancing an immune response.

In another aspect, the invention provides an immunogenic composition comprising: a first Pf bacteriophage peptide, or derivative thereof; a second Pf bacteriophage peptide, or derivative thereof; wherein the second Pf bacteriophage peptide, or derivative thereof, is different from the first bacteriophage peptide or derivative thereof; and one or more carriers; wherein the Pf bacteriophage peptides, or derivatives thereof, are independently coupled to the one or more carriers. Another aspect of the invention provides an immunogenic composition comprising: a first conjugate comprising a first Pf bacteriophage peptide, or derivative, coupled to a first carrier; and a second conjugate comprising a second Pf bacteriophage peptide, or derivative, coupled to a second carrier; wherein the first and second carriers may be the same or different; wherein the first Pf bacteriophage peptide, or derivative, and the second Pf bacteriophage peptide, or derivative, are different and have low sequence identity, such as in different phylogenetically-defined bacteriophage clades. The invention further provides a method of inducing or enhancing an immune response comprising administering to a subject in need thereof, an effective amount of the immunogenic composition, or a vaccine thereof. A further aspect provides the immunogenic composition, or vaccine thereof, for use in inducing or enhancing an immune response. Another aspect provides use of the immunogenic composition, or vaccine thereof, in the manufacture of a medicament for inducing or enhancing an immune response.

In other aspects, the invention provides vaccines and kits of the conjugates and compositions disclosed herein.

The methods of inducing or inhibiting an immune response may reduce or prevent Pseudomonas aeruginosa biofilm formation in a subject.

### DETAILED DESCRIPTION

### 1. Definitions

The term “alkyl” as used herein, means a straight or branched chain saturated hydrocarbon. Representative examples of alkyl include, but are not limited to, methyl, ethyl, npropyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” as used herein, means a divalent group derived from a straight or branched chain saturated hydrocarbon. Representative examples of alkylene include, but are not limited to, —CH₂—, —CH₂CH₂—, —CH₂CH₂CH₂—, —CH₂CH(CH₃)CH₂—, and CH₂CH(CH₃)CH(CH₃)CH₂—.

The term “alkenyl” means a straight or branched, hydrocarbon chain containing at least one carbon-carbon double bond.

The term “halogen” or “halo” means a chlorine, bromine, iodine, or fluorine atom.

The term “haloalkyl,” as used herein, means an alkyl, as defined herein, in which one, two, three, four, five, six, or seven hydrogen atoms are replaced by halogen. For example, representative examples of haloalkyl include, but are not limited to, 2-fluoroethyl, difluoromethyl, trifluoromethyl, 2,2,2-trifluoroethyl, 2,2,2-trifluoro-1, 1-dimethylethyl, and the like.

The term “cycloalkyl” as used herein, means a monocyclic all-carbon ring containing zero heteroatoms as ring atoms, and zero double bonds. Examples of cycloalkyls include, but are not limited to, cyclopropyl, cyclobutyl, cyclopentyl, cyclohexyl, cycloheptyl, and cyclooctyl. The cycloalkyl groups described herein can be appended to the parent molecular moiety through any substitutable carbon atom.

The term “phenylene” refers to a divalent group derived from a benzene by removal of two hydrogen atoms from the ortho, meta, or para positions. A phenylene is used as a spacer element in a linker structure.

The term “cycloalkylene” refers to a divalent group derived from a cycloalkane by removal of two hydrogen atoms. A cycloalkylene is used as a spacer element in a linker structure.

The term “oxo” as used herein refers to an oxygen atom bonded to the parent molecular moiety. An oxo may be attached to a carbon atom or a sulfur atom by a double bond. Alternatively, an oxo may be attached to a nitrogen atom by a single bond, i.e., an N-oxide.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

Certain compounds of the invention have the stereochemical configurations around the core sugar as specifically shown in formula (I) below. Apart from the core sugar stereochemistry, stereocenters located in any substituent appended to the core sugar include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Thus, included within the scope of the invention are tautomers of compounds of formula I. The structures also include zwitterioinc forms of the compounds or salts of formula I where appropriate.

A Pf bacteriophage peptide comprises a sequence of amino acids native to a coat protein of a filamentous bacteriophage. A Pf bacteriophage peptide derivative comprises a sequence of amino acids that is different from the wild-type sequence. Differences may include additions, deletions, or substitutions of one or more amino acids relative to the wild-type sequence. A Pf bacteriophage peptide derivative comprises a peptide having at least 70%, at least 75%, at least 80%, at least 85%, at least 90%, at least 94%, or at least about 95% sequence identity with a native sequence. Sequence identity (%) may be determined by alignment using the commercial algorithm ClustalW using default parameters (Pairwise Alignment: Alignment Speed=Slow, Open Gap Penalty=15, and Extend Gap Penalty=6.66; Multiple Alignment: Open Gap Penalty=15, and Extend Gap Penalty=6.66; Delay Divergent=30%, and Transitions=Weighted), as described by Thompson et al. in Current Protocols in Bioinformatics 2003, vol 00 (1) pp. 2.3.1-2.3.22 (doi.org/10.1002/0471250953.bi0203s00).

An acidic region of a CoaB coat protein of a Pf bacteriophage refers to a Pf bacteriophage peptide comprising one or more acidic amino acid residues.

The term “amino acid” refers to naturally occurring and synthetic/unnatural amino acids. Naturally occurring amino acids are those encoded by the genetic code as well as those amino acids that are later modified, e.g., hydroxyproline, γ-carboxyglutamate, and O-phosphoserine. For example, the term “natural amino acid” refers to any one of the common, naturally occurring L-amino acids found in proteins, including glycine (Gly), alanine (Ala), valine (Val), leucine (Leu), isoleucine (Ile), lysine (Lys), arginine (Arg), histidine (His), proline (Pro), serine (Ser), threonine (Thr), phenylalanine (Phe), tyrosine (Tyr), tryptophan (Trp), aspartic acid (Asp), glutamic acid (Glu), asparagine (Asn), glutamine (Gln), cysteine (Cys), and methionine (Met).

As used herein, the term “unnatural amino acid” refers to all amino acids that are not natural amino acids as described above. Such amino acids include the D-isomers of any of the naturally occurring amino acids described above. Unnatural amino acids also include homoserine, ornithine, norleucine, thyroxine, selenocysteine, selenomethionine, citrulline, penicillamine, mono-iodo-tyrosine. Unnatural amino acids as part of a carrier may contain an alkyne or azide group capable of undergoing cycloaddition with a corresponding functional group on a linker precursor to form a triazole (Click chemistry). Additional unnatural amino acids are well known to one of ordinary skill in the art and are available from commercial sources (e.g., www.sigmaaldrich.com/chemistry/chemistry-products.html?TablePage=16274965). An unnatural amino acid may be a D- or L-isomer. An unnatural amino acid may also be an alpha amino acid or a beta amino acid. An unnatural amino acid may also be a post-translationally modified amino acid such as a phosphorylated serine, threonine or tyrosine, an acylated lysine, or an alkylated lysine or arginine. Many forms of post-translationally modified amino acids are known. Other unnatural amino acids include 3-substituted alanine derivatives, glycine derivatives, N-methyl amino acids, ring-substituted phenylalanine and tyrosine derivatives, leucine/isoleucine derivatives, glutamic or aspartic acid derivatives (e.g., alpha-methyl glutamic acid, 3-methyl glutamic acid, 4-methyl glutamic acid, 4-fluoro glutamic acid, 1-(2-amino-2-carboxyethyl)cyclopropane-1-carboxylic acid, alpha-methyl aspartic acid, 3-methyl aspartic acid, 3-fluoro aspartic acid).

An “acidic amino acid” is an amino acid having an acidic side chain at neutral pH and includes, for example, glutamic acid and aspartic acid and derivatives thereof.

A “basic amino acid” is an amino acid having a basic side chain at neutral pH and includes, for example, lysine, arginine, and histidine, and derivatives thereof.

“A neutral amino acid” is an amino acid that is neither acidic or basic at neutral pH and includes, for example, glycine, proline, amino acids with hydrophobic side chains such as alanine, valine, leucine, isoleucine, phenylalanine, methionine, tryptophan, and tyrosine, and derivatives, and amino acids with polar neutral side chains such as asparagine, cysteine, glutamine, serine, and threonine, and derivatives.

The term “inducing” such as in the phrase “inducing an immune response” means directly or indirectly stimulating an immune response and/or enhancing an existing immune response to obtain a desired physiologic effect.

As used herein, the terms “administration” or “administering” typically refer to the step of introducing the vaccine and/or immunogenic compositions, as herein described, into a subject's body so that the subject's immune system mounts an immune response.

### 2. Immunogenic Conjugates and Compositions

In the following, embodiments of the invention are disclosed. The first embodiment is denoted E1, and other embodiments are denoted E1.1., E1.2, E2, etc.
E1. An immunogenic conjugate comprising two or more different Pf bacteriophage peptides, or derivatives thereof, coupled to a carrier.
E1.1. The immunogenic conjugate of E1 comprising two different Pf bacteriophage peptides, or derivatives thereof, coupled to the carrier.
E1.2. The immunogenic conjugate of E1 comprising three different Pf bacteriophage peptides, or derivatives thereof, coupled to the carrier.
E1.3. The immunogenic conjugate of E1 comprising four different Pf bacteriophage peptides, or derivatives thereof, coupled to the carrier.
E1.4. The immunogenic conjugate of E1 comprising five different Pf bacteriophage peptides, or derivatives thereof, coupled to the carrier.
E1.5. The immunogenic conjugate of E1 comprising six different Pf bacteriophage peptides, or derivatives thereof, coupled to the carrier.
E1.6. The immunogenic conjugate of E1 comprising seven different Pf bacteriophage peptides, or derivatives thereof, coupled to the carrier.
E1.7. The immunogenic conjugate of E1 comprising eight different Pf bacteriophage peptides, or derivatives thereof, coupled to the carrier.
E1.8. The immunogenic conjugate of E1 comprising nine different Pf bacteriophage peptides, or derivatives thereof, coupled to the carrier.
E1.9. The immunogenic conjugate of E1 comprising ten different Pf bacteriophage peptides, or derivatives thereof, coupled to the carrier.
E1.10. The immunogenic conjugate of any of E1-E1.9 comprising a plurality of each of the different Pf bacteriophage peptides, or derivatives thereof, coupled to the carrier.
E1.11. The immunogenic conjugate of any of E1-E1.10 having 2-100 Pf bacteriophage peptides, or derivatives thereof, coupled to the carrier.
E1.12. The immunogenic conjugate of any of E1-E1.10 having 2-20 Pf bacteriophage peptides, or derivatives thereof, coupled to the carrier.
E1.13. The immunogenic conjugate of E1.11 having 10-15 Pf bacteriophage peptides, or derivatives thereof, coupled to the carrier.
E2. An immunogenic composition comprising: an immunogenic conjugate comprising a Pf bacteriophage peptide, or a derivative thereof, coupled to a carrier; and one or more adjuvants, the one or more adjuvants comprising a TLR4 agonist and/or a TLR7/8 agonist.
E2.1. The immunogenic composition of E2, wherein the immunogenic conjugate comprises a plurality of Pf bacteriophage peptides, or derivatives thereof, coupled to the carrier.
E2.2. The immunogenic composition of E2.1, wherein the immunogenic conjugate has 2-100 or 2-20 Pf bacteriophage peptides, or derivatives thereof, coupled to the carrier.
E2.3. The immunogenic composition of E2.1, wherein the immunogenic conjugate has 10-15 Pf bacteriophage peptides, or derivatives thereof, coupled to the carrier.
E3. The immunogenic conjugate or immunogenic composition of any of E1-E2.3, wherein at least one of the Pf bacteriophage peptides is a Pf4 bacteriophage peptide.
E3.1. The immunogenic conjugate or immunogenic composition of any of E1-E3, wherein at least one of the Pf bacteriophage peptides is a Pf1 bacteriophage peptide.
E3.2. The immunogenic conjugate or immunogenic composition of any of E1-E3.1, wherein at least one of the Pf bacteriophage peptides is a Pf5 bacteriophage peptide.
E3.3. The immunogenic conjugate or immunogenic composition of any of E1-E3.2, wherein at least one of the Pf bacteriophage peptides is a Pf6 bacteriophage peptide.
E4. The immunogenic conjugate or immunogenic composition of any of E1-E3.3, wherein the Pf bacteriophage peptide comprises an acidic region of a CoaB coat protein of a Pf bacteriophage.
E5. The immunogenic conjugate or immunogenic composition of any of E1-E4, wherein each Pf bacteriophage peptide, or derivative thereof, independently comprises from 20-25% acidic residues.
E5.1. The immunogenic conjugate or immunogenic composition of E5, wherein the acidic residues are independently selected from aspartic acid and glutamic acid.
E5.2. The immunogenic conjugate or immunogenic composition of E5 or E5.1, wherein each Pf bacteriophage peptide, or derivative thereof, independently contains 2, 3, 4, or 5 acidic residues.
E6. The immunogenic conjugate or immunogenic composition of any of E1-E5.2, wherein each Pf bacteriophage peptide, or derivative thereof, forms an α-helix.
E6.1. The immunogenic conjugate or immunogenic composition of any of E1-E6, wherein one or more of the Pf bacteriophage peptides, or derivative thereof, has acidic residues at positions 2, 6, 11, and optionally position 16 from the point of attachment (i.e., the amino acid at the point of attachment to the linker is position 1).
E6.2. The immunogenic conjugate or immunogenic composition of any of E1-E6, wherein one or more of the Pf bacteriophage peptides, or derivative thereof, has acidic residues at positions 2, 14, 15, 19, and optionally position 10 from the point of attachment.
E6.3. The immunogenic conjugate or immunogenic composition of any of E1-E6, wherein one or more of the Pf bacteriophage peptides, or derivative thereof, has acidic residues at positions 7, 9, and 11 from the point of attachment.
E6.4. The immunogenic conjugate or immunogenic composition of any of E1-E6, wherein one or more of the Pf bacteriophage peptides, or derivative thereof, has acidic residues at positions 3, 7, 18, and optionally position 15 from the point of attachment.
E6.5. The immunogenic conjugate or immunogenic composition of any of E1-E6, wherein one or more of the Pf bacteriophage peptides, or derivative thereof, has acidic residues at positions 2, 6, 17, and optionally position 13 from the point of attachment.
E6.6. The immunogenic conjugate or immunogenic composition of any of E1-E6, wherein one or more of the Pf bacteriophage peptides, or derivative thereof, has acidic residues at positions 11 and 17 from the point of attachment.
E6.7. The immunogenic conjugate or immunogenic composition of any of E1-E6, wherein one or more of the Pf bacteriophage peptides, or derivative thereof, has acidic residues at positions 8, 10, and 17 from the point of attachment.
E6.8. The immunogenic conjugate or immunogenic composition of any of E1-E6, wherein one or more of the Pf bacteriophage peptides, or derivative thereof, has acidic residues at positions 14, 16, and 18 from the point of attachment.
E6.9. The immunogenic conjugate or immunogenic composition of any of E1-E6, wherein one or more of the Pf bacteriophage peptides, or derivative thereof, has acidic residues at positions 3, 7, and optionally 14 and/or 18 from the point of attachment.
E6.10. The immunogenic conjugate or immunogenic composition of any of E1-E6, wherein one or more of the Pf bacteriophage peptides, or derivative thereof, has acidic residues at positions 2, 6, and 17 from the point of attachment.
E6.11. The immunogenic conjugate or immunogenic composition of any of E1-E6, wherein one or more of the Pf bacteriophage peptides, or derivative thereof, has acidic residues at positions 2, 6, 13, and optionally 17 from the point of attachment.
E6.12. The immunogenic conjugate or immunogenic composition of any of E1-E6, wherein one or more of the Pf bacteriophage peptides, or derivative thereof, has acidic residues at positions 11 and 17 from the point of attachment.
E7. The immunogenic conjugate or immunogenic composition of any of E1-E6.12, wherein each Pf bacteriophage peptide, or derivative thereof, is between 5-45, 7-35, 12-25, or 14 to 22 amino acids in length.
E7.1. The immunogenic conjugate or immunogenic composition of E7, wherein at least one Pf bacteriophage peptide, or derivative thereof, is 14 amino acids in length.
E7.2. The immunogenic conjugate or immunogenic composition of E7 or E7.1, wherein at least one Pf bacteriophage peptide, or derivative thereof, is 15 amino acids in length.
E7.3. The immunogenic conjugate or immunogenic composition of E7-E7.2, wherein at least one Pf bacteriophage peptide, or derivative thereof, is 16 amino acids in length.
E7.4. The immunogenic conjugate or immunogenic composition of E7-E7.3, wherein at least one Pf bacteriophage peptide, or derivative thereof, is 17 amino acids in length.
E7.5. The immunogenic conjugate or immunogenic composition of E7-E7.4, wherein at least one Pf bacteriophage peptide, or derivative thereof, is 18 amino acids in length.
E7.6. The immunogenic conjugate or immunogenic composition of E7-E7.5, wherein at least one Pf bacteriophage peptide, or derivative thereof, is 19 amino acids in length.
E7.7. The immunogenic conjugate or immunogenic composition of E7-E7.6, wherein at least one Pf bacteriophage peptide, or derivative thereof, is 20 amino acids in length.
E7.8. The immunogenic conjugate or immunogenic composition of E7-E7.7, wherein at least one Pf bacteriophage peptide, or derivative thereof, is 21 amino acids in length.
E7.9. The immunogenic conjugate or immunogenic composition of E7-E7.8, wherein at least one Pf bacteriophage peptide, or derivative thereof, is 22 amino acids in length.
E7.10a. The immunogenic conjugate or immunogenic composition of E7, wherein at least one Pf bacteriophage peptide, or derivative, comprises or has formula (A)

A1-A2-A3-A4-A5-A6-A7-A8-A9-A10-A11-A12-A13-A14-A15-A16-A17-A18- (A)
wherein
A9, A14, and A18 are each independently an acidic amino acid residue;
A4 is an acidic or neutral amino acid residue;
A1-A3, A5-A8, A10-A13, A15, and A17 are each independently a neutral amino acid residue; and
A16 is a neutral or basic amino acid residue;
wherein the Pf bacteriophage peptide, or derivative, is attached at A18.
   A7.11a. The immunogenic conjugate or immunogenic composition of any of E7.10a, wherein
A1-A3, A5-A8, A10-A13, A15, and A17 are each independently a glycine, alanine, serine, valine, isoleucine, threonine, or glutamine residue; and
A16 is a glutamine or lysine residue.
   E7.12a. The immunogenic conjugate or immunogenic composition of E7.10a, wherein
A1 is a glycine, alanine, or serine residue;
A2 is a valine residue;
A3 is an isoleucine residue;
A5 is a threonine residue;
A6 is a serine residue;
A7 is an alanine or serine residue;
A8 is a valine residue;
A10 is a serine, alanine, or a glutamine residue;
A11 is an alanine residue;
A12 is an isoleucine residue;
A13 is a threonine residue;
A15 is a glycine residue;
A16 is a glutamine or lysine residue; and
A17 is a glycine or serine residue.
   E7.13a. The immunogenic conjugate or immunogenic composition of any of E7.10a-E7.12a, wherein A4 is an acidic amino acid residue.
   E7.14a. The immunogenic conjugate or immunogenic composition of any of E7.10a-E7.12a, wherein A4 is a neutral amino acid residue.
   E7.15a. The immunogenic conjugate or immunogenic composition of any of E7.10a-E7.14a, wherein A9, A14, and A18 are each independently an aspartic acid or glutamic acid residue.
   E7.16a. The immunogenic conjugate or immunogenic composition of any of E7.10a-E7.15a, wherein A9 is a glutamic acid residue.
   E7.17a. The immunogenic conjugate or immunogenic composition of any of E7.10a-E7.16a, wherein A14 is an aspartic acid residue.
   E7.18a. The immunogenic conjugate or immunogenic composition of any of E7.10a-E7.16a, wherein A14 is glutamic acid residue.
   E7.19a. The immunogenic conjugate or immunogenic composition of any of E7.10a-E7.18a, wherein A18 is an aspartic acid residue.
   E7.20a. The immunogenic conjugate or immunogenic composition of any of E7.10a-E7.19a, wherein A4 is an aspartic acid residue.
   E7.21a. The immunogenic conjugate or immunogenic composition of any of E7.10a-E7.19a, wherein A4 is a glycine residue.
   E7.10b. The immunogenic conjugate or immunogenic composition of E7, wherein at least one Pf bacteriophage peptide, or derivative, comprises or has formula (A-1)

A1-A2-A3-A4-A5-A6-A7-A8-A9-A10-A11-A12-A13-A14-A15-A16-A17-A18-A19- (A-1)
wherein
A9, A14, and A18 are each independently an acidic amino acid residue;
A4 is an acidic or neutral amino acid residue;
A1-A3, A5-A8, A10-A13, A15, A17, and A19 are each independently a neutral amino acid residue; and
A16 is a neutral or basic amino acid residue;
wherein the Pf bacteriophage peptide, or derivative, is attached at A19.
   A7.11b. The immunogenic conjugate or immunogenic composition of E7.10b, wherein
A1-A3, A5-A8, A10-A13, A15, A17, and A19 are each independently a glycine, alanine, serine, valine, isoleucine, threonine, glutamine, or methionine residue; and
A16 is a glutamine or lysine residue.
   E7.12b. The immunogenic conjugate or immunogenic composition of E7.10b, wherein
A1 is a glycine, alanine, or serine residue;
A2 is a valine residue;
A3 is an isoleucine residue;
A5 is a threonine residue;
A6 is a serine residue;
A7 is an alanine or serine residue;
A8 is a valine residue;
A10 is a serine, alanine, or a glutamine residue;
A11 is an alanine residue;
A12 is an isoleucine residue;
A13 is a threonine residue;
A15 is a glycine residue;
A16 is a glutamine or lysine residue;
A17 is a glycine or serine residue;
and
A19 is a methionine residue.
   E7.13b. The immunogenic conjugate or immunogenic composition of any of E7.10b-E7.12b, wherein A4 is an acidic amino acid residue.
   E7.14b. The immunogenic conjugate or immunogenic composition of any of E7.10b-E7.12b, wherein A4 is neutral amino acid residue.
   E7.15b. The immunogenic conjugate or immunogenic composition of any of E7.10b-E7.14b, wherein A9, A14, and A18 are each independently an aspartic acid or glutamic acid residue.
   E7.16ba. The immunogenic conjugate or immunogenic composition of any of E7.10b-E7.15b, wherein A9 is an aspartic acid residue.
   E7.16bb. The immunogenic conjugate or immunogenic composition of any of E7.10b-E7.15b, wherein A9 is a glutamic acid residue.
   E7.17b. The immunogenic conjugate or immunogenic composition of any of E7.0b-E7.16bb, wherein A14 is an aspartic acid residue.
   E7.18b. The immunogenic conjugate or immunogenic composition of any of E7.10b-E7.16bb, wherein A14 is glutamic acid residue.
   E7.19ba. The immunogenic conjugate or immunogenic composition of any of E7.10b-E7.18b, wherein A18 is a glutamic acid residue.
   E7.19bb. The immunogenic conjugate or immunogenic composition of any of E7.10b-E7.18b, wherein A18 is an aspartic acid residue.
   E7.20b. The immunogenic conjugate or immunogenic composition of any of E7.10b-E7.19bb, wherein A4 is a glutamic acid residue.
   E7.20b. The immunogenic conjugate or immunogenic composition of any of E7.10b-E7.19bb, wherein A4 is an aspartic acid residue.
   E7.21b. The immunogenic conjugate or immunogenic composition of any of E7.10b-E7.19bb, wherein A4 is a glycine residue.
   E7.10c. The immunogenic conjugate or immunogenic composition of E7, wherein at least one Pf bacteriophage peptide, or derivative, comprises or has formula (A-2)

A0-A1-A2-A3-A4-A5-A6-A7-A8-A9-A10-A11-A12-A13-A14-A15-A16-A17-A18- (A-2)
wherein
A9, A14, and A18 are each independently an acidic amino acid residue;
A4 is an acidic or neutral amino acid residue;
A0-A3, A5-A8, A10-A13, A15, and A17 are each independently a neutral amino acid residue; and
A16 is a neutral or basic amino acid residue;
wherein the Pf bacteriophage peptide, or derivative, is attached at A18.

A7.11c. The immunogenic conjugate or immunogenic composition of E7.10c, wherein
A0-A3, A5-A8, A10-A13, A15, and A17 are each independently a glycine, alanine, serine, valine, isoleucine, threonine, or glutamine residue; and
A16 is a glutamine or lysine residue.
   E7.12c. The immunogenic conjugate or immunogenic composition of E7.10c, wherein
A0 is a glycine, alanine, or serine residue;
A1 is a glycine, alanine, or serine residue;
A2 is a valine residue;
A3 is an isoleucine residue;
A5 is a threonine residue;
A6 is a serine residue;
A7 is an alanine or serine residue;
A8 is a valine residue;
A10 is a serine, alanine, or a glutamine residue;
A11 is an alanine residue;
A12 is an isoleucine residue;
A13 is a threonine residue;
A15 is a glycine residue;
A16 is a glutamine or lysine residue; and
A17 is a glycine or serine residue.
   E7.13c. The immunogenic conjugate or immunogenic composition of any of E7.10c-E7.12c, wherein A4 is an acidic amino acid residue.
   E7.14c. The immunogenic conjugate or immunogenic composition of any of E7.10c-E7.12c, wherein A4 is neutral amino acid residue.
   E7.15c. The immunogenic conjugate or immunogenic composition of any of E7.10c-E7.14c, wherein A9, A14, and A18 are each independently an aspartic acid or glutamic acid residue.
   E7.16c. The immunogenic conjugate or immunogenic composition of any of E7.0c-E7.15c, wherein A9 is a glutamic acid residue.
   E7.17c. The immunogenic conjugate or immunogenic composition of any of E7.10c-E7.16c, wherein A14 is an aspartic acid residue.
   E7.18c. The immunogenic conjugate or immunogenic composition of any of E7.0c-E7.16c, wherein A14 is glutamic acid residue.
   E7.19c. The immunogenic conjugate or immunogenic composition of any of E7.10c-E7.18c, wherein A18 is an aspartic acid residue.
   E7.20c. The immunogenic conjugate or immunogenic composition of any of E7.10c-E7.19c, wherein A4 is an aspartic acid residue.
   E7.21c. The immunogenic conjugate or immunogenic composition of any of E7.10c-E7.19c, wherein A4 is a glycine residue.
   E7.22a. The immunogenic conjugate or immunogenic composition of any of E7-E7.21, wherein at least one Pf bacteriophage peptide, or derivative, comprises or has formula (B)

B2-B3-B4-B5-B6-B7-B8-B9-B10-B11-B12-B13-B14-B15-B16-B17-B18-B19-B20-B21- (B)
wherein
B3, B7, B8, and B20 are independently an acidic amino acid residue;
B12 is an acidic or neutral amino acid residue;
B2, B4-B6, B9, B10, B13-B19, and B21 are each independently a neutral amino acid residue; and
B11 is a basic amino acid residue;
wherein the Pf bacteriophage peptide, or derivative, is attached at B21.
   E7.23a. The immunogenic conjugate or immunogenic composition of E7.22a, wherein
B2, B4-B6, B9, B10, B13-B19, and B21 are each independently a phenylalanine, alanine, serine, leucine, isoleucine, threonine, valine, glutamine, or glycine residue; and
B11 is a lysine residue.
   E7.24a. The immunogenic conjugate or immunogenic composition of E7.22a, wherein
B2 is an alanine residue;
B4 is a serine residue;
B5 is a leucine residue;
B6 is a leucine or isoleucine residue;
B9 is threonine residue;
B10 is a threonine residue;
B11 is a lysine residue;
B13 is a valine or alanine residue;
B14 is a leucine residue;
B15 is a threonine or alanine residue;
B16 is a glutamine residue;
B17 is an alanine residue;
B18 is a glycine or serine residue;
B19 is a threonine residue; and
B21 is a glycine residue.
   E7.25a. The immunogenic conjugate or immunogenic composition of any of E7.22a-E7.24a, wherein B12 is an acidic amino acid residue.
   E7.26a. The immunogenic conjugate or immunogenic composition of any of E7.22a-E7.24a, wherein B12 is a neutral amino acid residue.
   E7.27a. The immunogenic conjugate or immunogenic composition of any of E7.22a-E7.26a, wherein B3, B7, B8, and B20 are each independently an aspartic acid or glutamic acid residue.
   E7.28. The immunogenic conjugate or immunogenic composition of any of E7.22-E7.27, wherein B3 is an aspartic acid residue.
   E7.29a. The immunogenic conjugate or immunogenic composition of any of E7.22a-E7.27a, wherein B3 is a glutamic acid residue.
   E7.30a. The immunogenic conjugate or immunogenic composition of any of E7.22a-E7.29a, wherein B7 is an aspartic acid residue.
   E7.31a. The immunogenic conjugate or immunogenic composition of any of E7.22a-E7.30a, wherein B8 is a glutamic acid residue.
   E7.32a. The immunogenic conjugate or immunogenic composition of any of E7.22a-E7.31a, wherein B20 is an aspartic acid residue.
   E7.33a. The immunogenic conjugate or immunogenic composition of any of E7.22a-E7.32a, wherein B12 is a glutamic acid residue.
   E7.34a. The immunogenic conjugate or immunogenic composition of any of E7.22a-E7.32a, wherein B12 is a glycine residue.
   E7.22b. The immunogenic conjugate or immunogenic composition of any of E7-E7.21, wherein at least one Pf bacteriophage peptide, or derivative, comprises or has formula (B-1)

B1-B2-B3-B4-B5-B6-B7-B8-B9-B10-B11-B12-B13-B14-B15-B16-B17-B18-B19-B20-B21- (B-1)
wherein
B3, B7, B8, and B20 are independently an acidic amino acid residue;
B12 is an acidic or neutral amino acid residue;
B1, B2, B4-B6, B9, B10, B13-B19, and B21 are each independently a neutral amino acid residue; and
B11 is a basic amino acid residue;
wherein the Pf bacteriophage peptide, or derivative, is attached at B21.
   E7.23b. The immunogenic conjugate or immunogenic composition of E7.22b, wherein
B1, B2, B4-B6, B9, B10, B13-B19, and B21 are each independently a phenylalanine, alanine, serine, leucine, isoleucine, threonine, valine, glutamine, or glycine residue; and
B11 is a lysine residue.
   E7.24b. The immunogenic conjugate or immunogenic composition of E7.22b, wherein
B1 is a phenylalanine residue;
B2 is an alanine residue;
B4 is a serine residue;
B5 is a leucine residue;
B6 is a leucine or isoleucine residue;
B9 is threonine residue;
B10 is a threonine residue;
B11 is a lysine residue;
B13 is a valine or alanine residue;
B14 is a leucine residue;
B15 is a threonine or alanine residue;
B16 is a glutamine residue;
B17 is an alanine residue;
B18 is a glycine or serine residue;
B19 is a threonine residue; and
B21 is a glycine residue.
   E7.25b. The immunogenic conjugate or immunogenic composition of any of E7.22b-E7.24b, wherein B12 is an acidic amino acid residue.
   E7.26b. The immunogenic conjugate or immunogenic composition of any of E7.22b-E7.24b, wherein B12 is a neutral amino acid residue.
   E7.27b. The immunogenic conjugate or immunogenic composition of any of E7.22b-E7.26b, wherein B3, B7, B8, and B20 are each independently an aspartic acid or glutamic acid residue.
   E7.28b. The immunogenic conjugate or immunogenic composition of any of E7.22b-E7.27b, wherein B3 is an aspartic acid residue.
   E7.29b. The immunogenic conjugate or immunogenic composition of any of E7.22b-E7.27b, wherein B3 is a glutamic acid residue.
   E7.30b. The immunogenic conjugate or immunogenic composition of any of E7.22b-E7.29b, wherein B7 is an aspartic acid residue.
   E7.31b. The immunogenic conjugate or immunogenic composition of any of E7.22b-E7.30b, wherein B8 is a glutamic acid residue.
   E7.32b. The immunogenic conjugate or immunogenic composition of any of E7.22b-E7.31b, wherein B20 is an aspartic acid residue.
   E7.33b. The immunogenic conjugate or immunogenic composition of any of E7.22b-E7.32b, wherein B12 is a glutamic acid residue.
   E7.34b. The immunogenic conjugate or immunogenic composition of any of E7.22b-E7.32b, wherein B12 is a glycine residue.
   E7.35. The immunogenic conjugate or immunogenic composition of any of E7-E7.34, wherein at least one Pf bacteriophage peptide, or derivative, comprises or has formula (C1)

C1a-C2a-C3a-C4a-C5a-C6a-C7a-C8a-C9a-C10a-C11a-C12a-C13a-C14a- (C1)
wherein
C4a, C6a, and C8a are independently an acidic amino acid residue;
C11a is a basic amino acid residue; and
C1a-C3a, C5a, C7a, C9a, C10a, and C12a-C14 are independently a neutral amino acid residue;
wherein the Pf bacteriophage peptide, or derivative, is attached at C14a.
   E7.36. The immunogenic conjugate or immunogenic composition of E7.35, wherein
C1a-C3a, C5a, C7a, C9a, C10a, and C12a-C14a are each independently a methionine, alanine, glycine, isoleucine, threonine, or leucine residue; and
C11a is a lysine residue.
   E7.37. The immunogenic conjugate or immunogenic composition of E7.35, wherein
C1a is a methionine residue;
C2a is an alanine residue;
C3a is an alanine residue;
C5a a glycine residue;
C7a is an isoleucine residue;
C9a is a threonine residue;
C10a is a threonine residue;
C11a is a lysine residue;
C12a is an alanine residue;
C13a is a leucine residue; and
C14a is an alanine residue.
   E7.38. The immunogenic conjugate or immunogenic composition of any of E7.35-E7.37, wherein C4a is a glutamic acid residue.
   E7.39. The immunogenic conjugate or immunogenic composition of any of E7.35-E7.38, wherein C6a is an aspartic acid residue.
   E7.40. The immunogenic conjugate or immunogenic composition of any of E7.35-E7.39, wherein C8a is an aspartic acid residue.
   E7.41. The immunogenic conjugate or immunogenic composition of any of E7-E7.40, wherein at least one Pf bacteriophage peptide, or derivative, comprises or has formula (C2)

C1b-C2b-C3b-C4b-C5b-C6b-C7b-C8b-C9b-C10b-C11b-C12b-C13b-C14b-C15b-C16b-C17b-C18b-C19b-C20b- (C2)
wherein
C3b, C14b, and C18b are independently an acidic amino acid residue;
C1b, C2b, C4b, C5b, C7b-C13b, C15b, C19b, and C20b are independently a neutral amino acid residue;
C6b is an acidic or neutral amino acid residue; and
C16b and C17b are independently a neutral or basic amino acid residue;
wherein the Pf bacteriophage peptide, or derivative, is attached at C20b.
   E7.42. The immunogenic conjugate or immunogenic composition of E7.41, wherein
C1b, C2b, C4b, C5b, C7b-C13b, C15b, C19b, and C20b are each independently a methionine, alanine, valine, proline, threonine, leucine, or glycine residue; and
C16b and C17b are independently a glutamine, alanine, or lysine residue.
   E7.43. The immunogenic conjugate or immunogenic composition of E7.41, wherein
C1b is a methionine residue;
C2b is an alanine residue;
C4b is a valine residue;
C5b is a proline residue;
C7b is an alanine residue;
C8b is an alanine residue;
C9b is a threonine residue;
C10b is an alanine or threonine residue;
C11b is an alanine residue;
C12b is a leucine residue;
C13b is a threonine residue;
C15b is an alanine residue;
C16b is a glutamine or lysine residue;
C17b is an alanine or lysine residue;
C19b is a glycine residue; and
C20b is a valine residue.
   E7.44. The immunogenic conjugate or immunogenic composition of any of E7.41-E7.43, wherein C6b is an acidic amino acid residue.
   E7.45. The immunogenic conjugate or immunogenic composition of any of E7.41-E7.43, wherein C6b is neutral amino acid residue.
   E7.46. The immunogenic conjugate or immunogenic composition of any of E7.41-E7.45, wherein C3b, C14b, and C18b are each independently an aspartic acid or glutamic acid residue.
   E7.47. The immunogenic conjugate or immunogenic composition of any of E7.41-E7.46, wherein C3b is a glutamic acid residue.
   E7.48. The immunogenic conjugate or immunogenic composition of any of E7.41-E7.47, wherein C14b is an aspartic acid residue;
   E7.49. The immunogenic conjugate or immunogenic composition of any of E7.41-E7.47, wherein C14b is a glutamic acid residue.
   E7.50. The immunogenic conjugate or immunogenic composition of any of E7.41-E7.49, wherein C18b is an aspartic acid residue.
   E7.51. The immunogenic conjugate or immunogenic composition of any of E7.41-E7.50, wherein C6b is a glutamic acid residue.
   E7.52. The immunogenic conjugate or immunogenic composition of any of E7.41-E7.50, wherein C6b is an alanine residue.
   E7.53. The immunogenic conjugate or immunogenic composition of any of E7-E7.52, wherein at least one Pf bacteriophage peptide, or derivative, comprises or has formula (C3)

C1c-C2c-C3c-C4c-C5c-C6c-C7c-C8c-C9c-C10c-C11c-C12c-C13c-C14c-C15c-C16c-C17c-C18c-C19c- (C3)
wherein
C1c is a methionine residue;
C2c is an alanine residue;
C3c is an acidic amino acid residue;
C4c is a valine residue;
C5c is a proline residue;
C6c is an alanine residue;
C7c is an acidic amino acid residue or an alanine residue;
C8c is an alanine residue;
C9c is a threonine residue;
C10c is an alanine, threonine, or serine residue;
C11c is an alanine residue;
C12c is a leucine residue;
C13c is an alanine or threonine residue;
C14c is an acidic amino acid residue;
C15c is an alanine residue;
C16c is a lysine or glutamine residue;
C17c is a lysine or alanine residue;
C18c is an acidic amino acid residue; and
C19c is a glycine residue;
wherein the Pf bacteriophage peptide, or derivative, is attached at C19c.
   E7.54. The immunogenic conjugate or immunogenic composition of E7.53, wherein C3c is a glutamic acid residue.
   E7.55. The immunogenic conjugate or immunogenic composition of E7.53 or E7.54, wherein C7c is a glutamic acid residue.
   E7.56. The immunogenic conjugate or immunogenic composition of E7.53 or E7.54, wherein C7c is an aspartic acid residue.
   E7.57. The immunogenic conjugate or immunogenic composition of E7.53 or E7.54, wherein C7c is an alanine residue.
   E7.58. The immunogenic conjugate or immunogenic composition of any of E7.53-E7.57, wherein C14c is an aspartic acid residue.
   E7.59. The immunogenic conjugate or immunogenic composition of any of E7.53-E7.58, wherein C18c is an aspartic acid residue.
   E7.60. The immunogenic conjugate or immunogenic composition of any of E7-E7.59, wherein at least one Pf bacteriophage peptide, or derivative, comprises or has formula (C4)

C1d-C2d-C3d-C4d-C5d-C6d-C7d-C8d-C9d-C10d-C11d-C12d-C13d-C14d-C15d-C16d-C17d-C18d-C19d- (C4)
wherein
C1d is a methionine residue;
C2d is an alanine residue;
C3d is an acidic amino acid residue;
C4d is an isoleucine residue;
C5d is a phenyl alanine residue;
C6d is a glycine residue;
C7d is an alanine residue;
C8d is a valine residue;
C9d is an acid amino acid residue;
C10d is a phenyl alanine residue;
C11d is a serine residue;
C12d is a serine residue;
C13d is a valine residue;
C14d is a serine residue;
C15d is a threonine residue;
C16d is a tryptophan residue;
C17d is a valine residue;
C18d is a valine residue; and
C19d is a serine residue;
wherein the Pf bacteriophage peptide, or derivative, is attached at C19d.
E7.61. The immunogenic conjugate or immunogenic composition of E7.60, wherein C3d is an aspartic acid residue.
E7.62. The immunogenic conjugate or immunogenic composition of E7.60 or E7.61, wherein C9d is an aspartic acid residue.
E7.63. The immunogenic conjugate or immunogenic composition of any of E7-E7.62, wherein at least one Pf bacteriophage peptide, or derivative, comprises or has formula (D1)

D1a-D2a-D3a-D4a-D5a-D6a-D7a-D8a-D9a-D10a-D11a-D12a-D13a-D14a-D15a-D16a-D17a-D18a-D19a-D20a- (D1)
wherein
D1a is an alanine residue;
D2a is a glycine residue;
D3a is a tryptophan residue;
D4a is an acid amino acid residue;
D5a is a tyrosine residue;
D6a is a serine residue;
D7a is a serine, glycine, threonine, or valine residue;
D8a is a leucine or methionine residue.
D9a is a threonine residue;
D10a is a serine, lysine, or glycine residue;
D11a is an acidic amino acid residue;
D12a is a valine or isoleucine residue;
D13a is an acidic amino acid residue;
D14a is a phenyl alanine or tyrosine residue;
D15a is a serine or glycine residue;
D16a is a threonine residue;
D17a is an isoleucine or valine residue;
D18a is an alanine residue;
D19a is a threonine residue; and
D20a is a glycine or serine residue;
wherein the Pf bacteriophage peptide, or derivative, is attached at D20a.
   E7.64. The immunogenic conjugate or immunogenic composition of E7.63, wherein D4a is an aspartic acid residue.
   E7.65. The immunogenic conjugate or immunogenic composition of E7.63 or E7.64, wherein D11a is an aspartic acid residue.
   E7.66. The immunogenic conjugate or immunogenic composition of any of E7.63-E7.65, wherein D13a is an aspartic acid residue.
   E7.67. The immunogenic conjugate or immunogenic composition of any of E7-E7.66, wherein at least one Pf bacteriophage peptide, or derivative, comprises or has formula (D2)

D1b-D2b-D3b-D4b-D5b-D6b-D7b-D8b-D9b-D10b-D11b-D12b-D13b-D14b-D15b-D16b-D17b-D18b-D19b- (D2)
wherein
D1b is an alanine residue;
D2b is an acidic amino acid residue;
D3b is a glycine residue;
D4b is an acid amino acid residue;
D5b is a leucine residue;
D6b is an acid amino acid residue;
D7b is a phenyl alanine residue;
D8b is a serine residue.
D9b is a serine residue;
D10b is a methionine residue;
D11b is an isoleucine residue;
D12b is a serine residue;
D13b is a serine residue;
D14b is a valine residue;
D15b is an asparagine residue;
D16b is a glycine residue;
D17b is a serine residue;
D18b is an alanine residue; and
D19b is a valine residue;
wherein the Pf bacteriophage peptide, or derivative, is attached at D19b.
   E7.68. The immunogenic conjugate or immunogenic composition of E7.67, wherein D2b is a glutamic acid residue.
   E7.69. The immunogenic conjugate or immunogenic composition of E7.67 or E7.68, wherein D4b is an aspartic acid residue.
   E7.70. The immunogenic conjugate or immunogenic composition of any of E7.67-E7.69, wherein D6b is an aspartic acid residue.

In E7.0a-E7.70, the position of attachment is a designation of which end of the sequence formula (e.g., formula (A)) attaches to the remainder of the conjugate. A Pf bacteriophage peptide that comprises the sequence formula may include still other amino acid residues on either end of the sequence formula.
E7.100a. The immunogenic conjugate or immunogenic composition of any of E1-E2.1, wherein at least one Pf bacteriophage peptide, or derivative comprises, or consists of, a peptide independently selected from the group consisting of SEQ ID NO: 1 to SEQ ID NO: 33.

| SEQ ID # | Peptide Sequence |
|---|---|
| SEQ ID NO: 1 | MAAVPAEATSALTEAKADGV |
| SEQ ID NO: 2 | MAEVPAAATTALTDAQKDGV |
| SEQ ID NO: 3 | GVIDTSAVESAITDGQGDM |
| SEQ ID NO: 4 | GVIDTSAVEAAITEGKGDM |
| SEQ ID NO: 5 | GVIGTSAVESAITDGQGDM |
| SEQ ID NO: 6 | GVIDTSAVEAAITDGQGDM |
| SEQ ID NO: 7 | AVIDTSAVESAITDGQGDM |
| SEQ ID NO: 8 | SVIDTSSVEQAITDGKSDM |
| SEQ ID NO: 9 | GVIGAVALALAVGDGDM |
| SEQ ID NO: 10 | MAAEGDIDTTKALA |
| SEQ ID NO: 11 | AGWDYSSLTSDVDESTIATG |
| SEQ ID NO: 12 | AGWDYSSLTSDVDESTIATS |
| SEQ ID NO: 13 | AGWDYSSMTSDVDESTIATG |
| SEQ ID NO: 14 | AGWDYSTLTSGIDEGTIATG |
| SEQ ID NO: 15 | AGWDYSVLTSDVDEGTIATG |
| SEQ ID NO: 16 | AGWDYSSLTSDVDYSTVATG |
| SEQ ID NO: 17 | AGWDYSGLTSDIDESTIATG |
| SEQ ID NO: 18 | AGWDYSGMTKDVDESTIATG |
| SEQ ID NO: 19 | AGWDYSGLTGDIDESTIATG |
| SEQ ID NO: 20 | FADSLIDETTKEVLTQAGTDG |
| SEQ ID NO: 21 | FAESLLDETTKGVLAQASTDG |
| SEQ ID NO: 22 | ESLLDETTKGVLAQASTDG |
| SEQ ID NO: 23 | FAESLLDETTKGALAQASTDG |
| SEQ ID NO: 24 | FAESLIDETTKEVLTQAGTDG |
| SEQ ID NO: 25 | FAESLLDETTKEVLTQAGTDG |
| SEQ ID NO: 26 | MADIFGAVDESSVSTWVVS |
| SEQ ID NO: 27 | MAEVPAAATTALADAQKDG |
| SEQ ID NO: 28 | MAEVPAAATTALTDAQKDG |
| SEQ ID NO: 29 | MAEVPAAATTALTDAQRDG |
| SEQ ID NO: 30 | MAEVPADATAALADAKADG |
| SEQ ID NO: 31 | MAAVPAEATSALTEAKADG |
| SEQ ID NO: 32 | AEGDLDESSMISSVNGSAV |
| SEQ ID NO: 33 | SAAVPEEATKALTTAGTDV |

E7.100b. The immunogenic conjugate or immunogenic composition of any of E1-E2.1, wherein at least one Pf bacteriophage peptide, or derivative comprises, or consists of, a peptide independently selected from the group consisting of SEQ ID NO: 39 to SEQ ID NO: 56.

| SEQ ID # | Peptide Sequence |
|---|---|
| SEQ ID NO: 39 | GSVIDTSAVESAITDGQGDM |
| SEQ ID NO: 40 | GVIDTSAVESAITEGQGDM |
| SEQ ID NO: 41 | GSVIDTSAVEAAITEGKGDM |
| SEQ ID NO: 42 | GSVIDTSAVESAITDGQGD |
| SEQ ID NO: 43 | GVIDTSAVESAITDGQGD |
| SEQ ID NO: 44 | GSVIDTSAVEAAITEGKGD |
| SEQ ID NO: 45 | AESLLDETTKEVLTQAGTDG |
| SEQ ID NO: 46 | ADSLIDETTKEVLTQAGTDG |
| SEQ ID NO: 47 | AESLIDETTKGVLAQASTDG |
| SEQ ID NO: 48 | DSLIDETTKEVLTQAGTDG |
| SEQ ID NO: 49 | AESLLDETTKGVLAQASTDG |
| SEQ ID NO: 50 | DYSSLTSDVDESTIATG |
| SEQ ID NO: 51 | GWDYSSLTSDVDESTIATG |
| SEQ ID NO: 52 | AEGDIDTTKALAYIAGGLT |
| SEQ ID NO: 53 | CESLLDETTKGVLAQASTDG |
| SEQ ID NO: 54 | ESLLDETTKCVLAQASTDG |
| SEQ ID NO: 55 | DSLIDETTKEVLTQAGTDGK |
| SEQ ID NO: 56 | GVIDTSAVESAITDGQGDMK |

E7.100c. The immunogenic conjugate or immunogenic composition of any of E1-E2.1, wherein at least one Pf bacteriophage peptide, or derivative comprises, or consists of, a peptide independently selected from the group consisting of SEQ ID NO: 3-4, 6-8 and 39-44.

| SEQ ID # | Peptide Sequence |
|---|---|
| SEQ ID NO: 3 | GVIDTSAVESAITDGQGDM |
| SEQ ID NO: 4 | GVIDTSAVEAAITEGKGDM |
| SEQ ID NO: 6 | GVIDTSAVEAAITDGQGDM |
| SEQ ID NO: 7 | AVIDTSAVESAITDGQGDM |
| SEQ ID NO: 8 | SVIDTSSVEQAITDGKSDM |
| SEQ ID NO: 39 | GSVIDTSAVESAITDGQGDM |
| SEQ ID NO: 40 | GVIDTSAVESAITEGQGDM |
| SEQ ID NO: 41 | GSVIDTSAVEAAITEGKGDM |
| SEQ ID NO: 42 | GSVIDTSAVESAITDGQGD |
| SEQ ID NO: 43 | GVIDTSAVESAITDGQGD |
| SEQ ID NO: 44 | GSVIDTSAVEAAITEGKGD |

E7.100d. The immunogenic conjugate or immunogenic composition of any of E1-E2.1, wherein at least one Pf bacteriophage peptide, or derivative comprises, or consists of, a peptide independently selected from the group consisting of SEQ ID NO: 20-25 and 45-47.

| SEQ ID # | Peptide Sequence |
|---|---|
| SEQ ID NO: 20 | FADSLIDETTKEVLTQAGTDG |
| SEQ ID NO: 21 | FAESLLDETTKGVLAQASTDG |
| SEQ ID NO: 22 | ESLLDETTKGVLAQASTDG |
| SEQ ID NO: 23 | FAESLLDETTKGALAQASTDG |
| SEQ ID NO: 24 | FAESLIDETTKEVLTQAGTDG |
| SEQ ID NO: 25 | FAESLLDETTKEVLTQAGTDG |
| SEQ ID NO: 45 | AESLLDETTKEVLIQAGTDG |
| SEQ ID NO: 46 | ADSLIDETTKEVLTQAGTDG |
| SEQ ID NO: 47 | AESLIDETTKGVLAQASTDG |

E7.101. The immunogenic conjugate or immunogenic composition of any of E1-E2.1, wherein the Pf bacteriophage peptide, or derivative, at each occurrence, comprises, or consists of, a peptide independently selected from the group consisting of SEQ ID NO: 1 to SEQ ID NO: 33.
E7.102. The immunogenic conjugate or immunogenic composition of any of E1-E2.1, wherein the conjugate comprises a Pf bacteriophage peptide, or derivative, comprising, or consisting of, any of SEQ ID NO: 3 to SEQ ID NO: 9.
E7.103. The immunogenic conjugate or immunogenic composition of E7.102, wherein the conjugate comprises a Pf bacteriophage peptide, or derivative, comprising, or consisting of, SEQ ID NO: 3.
E7.104. The immunogenic conjugate or immunogenic composition of any of E7.102 or E7.103, wherein the conjugate comprises a Pf bacteriophage peptide, or derivative, comprising, or consisting of, SEQ ID NO: 4.
E7.105. The immunogenic conjugate or immunogenic composition of any of E1-E2.1 or E7.102-E7.104, wherein the conjugate comprises a Pf bacteriophage peptide, or derivative, comprising, or consisting of, any of SEQ ID NO: 20 to SEQ ID NO: 25.
E7.106. The immunogenic conjugate or immunogenic composition of E7.105, wherein the conjugate comprises a Pf bacteriophage peptide, or derivative, comprising, or consisting of SEQ ID NO: 21.
E7.107. The immunogenic conjugate or immunogenic composition of E7.105, wherein the conjugate comprises a Pf bacteriophage peptide, or derivative, comprising, or consisting of SEQ ID NO: 22.
E7.108. The immunogenic conjugate or immunogenic composition of any of E1-E2.1 or E7.102-E7.107, wherein the conjugate comprises a Pf bacteriophage peptide, or derivative, comprising, or consisting of, SEQ ID NO: 10.
E7.109. The immunogenic conjugate or immunogenic composition of any of E1-E2.1 or E7.102-E7.108, wherein the conjugate comprises a Pf bacteriophage peptide, or derivative, comprising, or consisting of, any of SEQ ID NO: 1 to SEQ ID NO: 2.
E7.110. The immunogenic conjugate or immunogenic composition of any of E1-E2.1 or E7.102-E7.109, wherein the conjugate comprises a Pf bacteriophage peptide, or derivative, comprising, or consisting of, any of SEQ ID NO: 27 to SEQ ID NO: 31.
E7.111. The immunogenic conjugate or immunogenic composition of any of E1-E2.1 or E7.102-E7.110, wherein the conjugate comprises a Pf bacteriophage peptide, or derivative, comprising, or consisting of, SEQ ID NO: 26.
E7.112. The immunogenic conjugate or immunogenic composition of any of E1-E2.1 or E7.102-E7.111, wherein the conjugate comprises a Pf bacteriophage peptide, or derivative, comprising, or consisting of, any of SEQ ID NO: 11 to SEQ ID NO: 19.
E7.113. The immunogenic conjugate or immunogenic composition of any of E1-E2.1 or E7.102-E7.112, wherein the conjugate comprises a Pf bacteriophage peptide, or derivative, comprising, or consisting of, SEQ ID NO: 32.
E7.114. The immunogenic conjugate or immunogenic composition of any of E1-E2.1 or E7.102-E7.113, wherein the conjugate comprises a Pf bacteriophage peptide, or derivative, comprising, or consisting of, SEQ ID NO: 33.
E7.115. The immunogenic conjugate or immunogenic composition of any of E1-E2.1, wherein the conjugate comprises first and second Pf bacteriophage peptides, or derivatives, the first Pf bacteriophage peptide or derivative comprising, or consisting of, any of SEQ ID NO: 3 to SEQ ID NO: 9 and the second Pf bacteriophage peptide or derivative comprising, or consisting of, any of SEQ ID NO: 20 to SEQ ID NO: 25.
E7.116. The immunogenic conjugate or immunogenic composition of any of E1-E2.1, wherein the conjugate comprises first, second, and third Pf bacteriophage peptides, or derivatives, the first Pf bacteriophage peptide or derivative comprising, or consisting of, any of SEQ ID NO: 3 to SEQ ID NO: 9, the second Pf bacteriophage peptide or derivative comprising, or consisting of, any of SEQ ID NO: 20 to SEQ ID NO: 25, and the third bacteriophage peptide, or derivative, comprising, or consisting of, any of SEQ ID NO: 3 to SEQ ID NO: 9 different from the first bacteriophage peptide, or derivative.
E7.117. The immunogenic conjugate or immunogenic composition of any of E1-E2.1, wherein the conjugate comprises first and second Pf bacteriophage peptides, or derivatives, the first Pf bacteriophage peptide or derivative comprising, or consisting of, SEQ ID NO: 3 and the second Pf bacteriophage peptide or derivative comprising, or consisting of, SEQ ID NO: 21.
E7.118. The immunogenic conjugate or immunogenic composition of any of E1-E2.1, wherein the conjugate comprises first, second, and third Pf bacteriophage peptides or derivatives, the first Pf bacteriophage peptide or derivative comprising, or consisting of, SEQ ID NO: 3, the second Pf bacteriophage peptide or derivative comprising, or consisting of, SEQ ID NO: 21, and the third Pf bacteriophage peptide or derivative comprising, or consisting of SEQ ID NO: 4.
E7.119. The immunogenic conjugate or immunogenic composition of any of E1-E2.1, wherein the conjugate comprises first and second Pf bacteriophage peptides, or derivatives, the first Pf bacteriophage peptide or derivative comprising, or consisting of, SEQ ID NO: 3 and the second Pf bacteriophage peptide or derivative comprising, or consisting of, SEQ ID NO: 22.
E7.120. The immunogenic conjugate or immunogenic composition of any of E1-E2.1, wherein the conjugate comprises first, second, and third Pf bacteriophage peptides or derivatives, the first Pf bacteriophage peptide or derivative comprising, or consisting of, SEQ ID NO: 3, the second Pf bacteriophage peptide or derivative comprising, or consisting of, SEQ ID NO: 22, and the third Pf bacteriophage peptide or derivative comprising, or consisting of SEQ ID NO: 4.
E7.204. The immunogenic conjugate or immunogenic composition of any of E1-E2.1, wherein at least one Pf bacteriophage peptide or derivative, comprises, or consists of, a peptide independently selected from the group consisting of SEQ ID NO: 34 to SEQ ID NO: 35:

| SEQ ID # | Peptide Sequence |
|---|---|
| SEQ ID NO: 34 | DTSAVEX¹AITX²GX³GD |
| SEQ ID NO: 35 | X⁴SLX⁵DETTKX⁶VLX⁷QAX⁸TD |

wherein
X¹, X⁵, X⁷, and X⁸ are independently a neutral amino acid residue;
X² and X⁴ and are independently an acidic amino acid residue;
X³ is a neutral or basic amino acid residue; and
X⁶ is a neutral or acidic amino acid residue.
   E7.205. The immunogenic conjugate or immunogenic composition of E7.204, wherein
X¹ is a serine, alanine, or glutamine residue;
X² is an aspartic acid or glutamic acid residue;
X³ is a glutamine or lysine residue;
X⁴ is an aspartic acid or glutamic acid residue;
X⁵ is a leucine or isoleucine residue;
X⁶ is glycine or glutamic acid residue;
X⁷ is threonine or alanine residue; and
X⁸ is glycine or serine residue.
   E7.206. The immunogenic conjugate or immunogenic composition of E7.204 or E7.205, wherein the Pf bacteriophage peptide, or derivative, at each occurrence, comprises, or consists of, a peptide independently selected from the group consisting of SEQ ID NO: 34 and SEQ ID NO: 35.
   E7.207. The immunogenic conjugate or immunogenic composition of E7.204 or E7.205, wherein the conjugate comprises a Pf bacteriophage peptide, or derivative, comprising, or consisting of, SEQ ID NO: 34.
   E7.208. The immunogenic conjugate or immunogenic composition of any of E7.204-E7.207, wherein the conjugate comprises a Pf bacteriophage peptide, or derivative, comprising, or consisting of, SEQ ID NO:35.
   E7.209. The immunogenic conjugate or immunogenic composition of E7.204 or E7.205, wherein the conjugate comprises first and second Pf bacteriophage peptides, or derivatives, the first Pf bacteriophage peptide or derivative comprising, or consisting of, SEQ ID NO: 34 and the second Pf bacteriophage peptide or derivative comprising, or consisting of, SEQ ID NO: 35.
   E7.210. The immunogenic conjugate or immunogenic composition of E7.204 or E7.205, wherein at least one Pf bacteriophage peptide or derivative, comprises, or consists of, a peptide independently selected from the group consisting of SEQ ID NO: 34.
   E7.211. The immunogenic conjugate or immunogenic composition of E7.210, wherein at least one Pf bacteriophage peptide or derivative, comprises, or consists of, a peptide independently selected from the group consisting of SEQ ID NO: 57:

| SEQ ID # | Peptide Sequence |
|---|---|
| SEQ ID NO: 57 | X^{a}X^{b}DTSAVEX¹AITX²GX³GD |

wherein X^{a} and X^{b} are each independently a neutral amino acid residue.
   E7.212. The immunogenic conjugate or immunogenic composition of E7.210, wherein at least one Pf bacteriophage peptide or derivative, comprises, or consists of, a peptide independently selected from the group consisting of SEQ ID NO: 58:

| SEQ ID # | Peptide Sequence |
|---|---|
| SEQ ID NO: 58 | X^{a}X^{b}DTSAVEX¹AITX²GX³GDM |

wherein X^{a} and X^{b} are each independently a neutral amino acid residue.
   E7.213. The immunogenic conjugate or immunogenic composition of E7.210, wherein at least one Pf bacteriophage peptide or derivative, comprises, or consists of, a peptide independently selected from the group consisting of SEQ ID NO: 59:

| SEQ ID # | Peptide Sequence |
|---|---|
| SEQ ID NO: 59 | YX^{a}X^{b}DTSAVEX¹AITX²GX³GD |

wherein Y, X^{a} and X^{b} are each independently a neutral amino acid residue.
   E7.214. The immunogenic conjugate or immunogenic composition of E7.213, wherein Y is a glycine, alanine, or serine residue.
   E7.215. The immunogenic conjugate or immunogenic composition of E7.213, wherein Y is a glycine-serine diamino acid residue.
   E7.216. The immunogenic conjugate or immunogenic composition of any of E7.211-E7.215, wherein X^{a} and X^{b} are each independently a valine or isoleucine residue.
   E7.217. The immunogenic conjugate or immunogenic composition of E7.216, wherein X^{a} is a valine residue and X^{b} is an isoleucine residue.
   E7.220. The immunogenic conjugate or immunogenic composition of any of E7.204, E7.205, or E7.210-E7.217, wherein at least one Pf bacteriophage peptide or derivative, comprises, or consists of, a peptide independently selected from the group consisting of SEQ ID NO: 35.
   E7.221. The immunogenic conjugate or immunogenic composition of E7.220 wherein at least one Pf bacteriophage peptide or derivative, comprises, or consists of, a peptide independently selected from the group consisting of SEQ ID NO: 60:

| SEQ ID # | Peptide Sequence |
|---|---|
| SEQ ID NO: 60 | X⁴SLX⁵DETTKX⁶VLX⁷QAX⁸TDG |

E7.222. The immunogenic conjugate or immunogenic composition of E7.220 wherein at least one Pf bacteriophage peptide or derivative, comprises, or consists of, a peptide independently selected from the group consisting of SEQ ID NO: 61:

| SEQ ID # | Peptide Sequence |
|---|---|
| SEQ ID NO: 61 | AX⁴SLX⁵DETTKX⁶VLX⁷QAX⁸TDG |

E7.230. An immunogenic composition comprising:
a first Pf bacteriophage peptide, or derivative thereof;
a second Pf bacteriophage peptide, or derivative thereof; wherein the second Pf bacteriophage peptide, or derivative thereof, is different from the first bacteriophage peptide or derivative thereof; and
one or more carriers;
wherein the Pf bacteriophage peptides, or derivatives thereof, are independently coupled to the one or more carriers.
   E7.231. The immunogenic composition of E7.230, comprising:
a first conjugate comprising the first Pf bacteriophage peptide, or derivative, coupled to a first carrier; and
a second conjugate comprising the second Pf bacteriophage peptide, or derivative, coupled to a second carrier.
   E7.232. The immunogenic composition of E7.230, comprising an immunogenic conjugate comprising the first and second Pf bacteriophage peptides, or derivatives thereof, coupled to a first carrier.
   E7.240. The immunogenic composition of any of E7.230-E7.232, wherein the first Pf bacteriophage peptide, or derivative, comprises, or consists of, a peptide defined according to the Pf bacteriophage peptide described in any of E3-E6.12.
   E7.241. The immunogenic composition of any of E7.230-E7.232, wherein the first Pf bacteriophage peptide, or derivative, comprises, or consists of, a peptide defined according to the Pf bacteriophage peptide described in any of E7.10a-E7.21c.
   E7.242a. The immunogenic composition of any of E7.230-E7.232, wherein the first Pf bacteriophage peptide, or derivative, comprises, or consists of, a peptide defined according to the Pf bacteriophage peptide described in E7.100c.
   E7.242b. The immunogenic composition of any of E7.230-E7.232, wherein the first Pf bacteriophage peptide, or derivative, comprises, or consists of, a peptide selected from the group consisting of SEQ ID NO: 42-44.
   E7.243. The immunogenic composition of any of E7.230-E7.232, wherein the first Pf bacteriophage peptide, or derivative, comprises, or consists of, a peptide defined according to the Pf bacteriophage peptide described in any of E7.210-E7.217.
   E7.244. The immunogenic composition of any of E7.230-E7.243, wherein the second Pf bacteriophage peptide, or derivative, comprises, or consists of, a peptide defined according to the Pf bacteriophage peptide described in any of E3-E6.12.
   E7.245. The immunogenic composition of any of E7.230-E7.243, wherein the second Pf bacteriophage peptide, or derivative, comprises, or consists of, a peptide defined according to the Pf bacteriophage peptide described in any of E7.22a-E7.34b.
   E7.246a. The immunogenic composition of any of E7.230-E7.243, wherein the second Pf bacteriophage peptide, or derivative, comprises, or consists of, a peptide defined according to the Pf bacteriophage peptide described in E7.100d.
   E7.246b. The immunogenic composition of any of E7.230-E7.243, wherein the second Pf bacteriophage peptide, or derivative, comprises, or consists of, a peptide selected from the group consisting of SEQ ID. NO: 45, 46, and 47.
   E7.246c. The immunogenic composition of any of E7.230-E7.243, wherein the second Pf bacteriophage peptide, or derivative, comprises, or consists of, a peptide selected from the group consisting of SEQ ID. NO: 46, 47, and 49.
   E7.247. The immunogenic composition of any of E7.230-E7.243, wherein the second Pf bacteriophage peptide, or derivative, comprises, or consists of, a peptide defined according to the Pf bacteriophage peptide described in any of E7.220-E7.222.
   E7.248. The immunogenic composition of any of E7.230-E7.232, wherein the first and second Pf bacteriophage peptides each independently comprise, or consist of, a peptide selected from the group consisting of SEQ ID NO: 1-33 and 39-56.
   E7.250. An immunogenic composition comprising:
a first conjugate comprising a first Pf bacteriophage peptide, or derivative, coupled to a first carrier; and
a second conjugate comprising a second Pf bacteriophage peptide, or derivative, coupled to a second carrier;
wherein the first and second carriers may be the same or different (i.e., same or different type of carrier);
wherein the first Pf bacteriophage peptide, or derivative, is selected from a Pf bacteriophage peptide described in any one of E6.1-E6.12;
wherein the second Pf bacteriophage peptide, or derivative, is selected from a Pf bacteriophage peptide described in a different embodiment than the first Pf bacteriophage peptide in any one of E6.1-E6.12.
For example, when the first Pf bacteriophage peptide is selected from E6.1, the second Pf bacteriophage peptide may be selected from any one of E6.2-E6.12.
   E7.251. The immunogenic composition of E7.250, wherein the first and second Pf bacteriophage peptides independently have the sequence length described in any of E7-E7.9.
   E7.260. An immunogenic composition comprising:
a first conjugate comprising a first Pf bacteriophage peptide, or derivative, coupled to a first carrier; and
a second conjugate comprising a second Pf bacteriophage peptide, or derivative, coupled to a second carrier;
wherein the first and second carriers may be the same or different (i.e., same or different type of carrier);
wherein the first Pf bacteriophage peptide, or derivative, comprises or consists of a Pf bacteriophage peptide of formula (A), (A-1), (A-2), (B), (B-1), (C1), (C2), (C3), (C4), (D1), or (D2), as described in any one of E7.0a-E7.70;
wherein the second Pf bacteriophage peptide, or derivative, comprises or consists of a Pf bacteriophage peptide of a different formula than the first Pf bacteriophage peptide among formulas (A), (A-1), (A-2), (B), (B-1), (C1), (C2), (C3), (C4), (D1), and (D2), as described in any one of E7.10a-E7.70.
   E7.261. The immunogenic composition of E7.260 further comprising a third conjugate comprising a third Pf bacteriophage peptide, or derivative, coupled to a third carrier, wherein the third carrier may be the same or different from the first or second carriers, wherein the third Pf bacteriophage peptide, or derivative, comprises or consists of a Pf bacteriophage peptide of formula (A), (A-1), (A-2), (B), (B-1), (C1), (C2), (C3), (C4), (D1), or (D2), as described in any one of E7.0a-E7.70.
   E.7.262. The immunogenic composition of E7.260 or E7.261, wherein the first Pf bacteriophage peptide, or derivative, comprises or consists of a Pf bacteriophage peptide of formula (A), (A-1), or (A-2), and the second Pf bacteriophage peptide, or derivative, comprises or consists of a Pf bacteriophage peptide of formula (B) or (B-1).
   E.7.263. The immunogenic composition of E7.261 or E7.262, wherein the third Pf bacteriophage peptide, or derivative, comprises or consists of a Pf bacteriophage peptide of formula (A), (A-1), or (A-2).
   E7.270. An immunogenic composition comprising:
a first conjugate comprising a first Pf bacteriophage peptide, or derivative, coupled to a first carrier; and
a second conjugate comprising a second Pf bacteriophage peptide, or derivative, coupled to a second carrier;
wherein the first and second carriers may be the same or different;
wherein the first Pf bacteriophage peptide, or derivative, comprises or consists of any of SEQ ID NO: 3 to SEQ ID NO: 9; and
the second Pf bacteriophage peptide, or derivative, comprises or consists of any of the following options:
any of SEQ ID NO: 1 to SEQ ID NO: 2;
SEQ ID NO: 10;
any of SEQ ID NO: 11 to SEQ ID NO: 19;
any of SEQ ID NO: 20 to SEQ ID NO: 25;
SEQ ID NO: 26;
any of SEQ ID NO: 27 to SEQ ID NO: 31;
SEQ ID NO: 32; or
SEQ ID NO: 33.
   E7.271. The immunogenic composition of E7.270 further comprising a third conjugate comprising a third Pf bacteriophage peptide, or derivative, coupled to a third carrier, wherein the third carrier may be the same or different from the first or second carriers, wherein the third Pf bacteriophage peptide, or derivative, comprises or consists of a Pf bacteriophage peptide of any of SEQ ID NO: 1 to SEQ ID NO: 33.
   E7.272. The immunogenic composition of E7.270 or E7.271, wherein the second Pf bacteriophage peptide, or derivative, comprises or consists of any of SEQ ID NO: 20 to SEQ ID NO: 25.
   E.7.273. The immunogenic composition of E7.271 or E7.272, wherein the third Pf bacteriophage peptide, or derivative, comprises or consists of any of SEQ ID NO: 3 to SEQ ID NO: 9, wherein the first and third Pf bacteriophage peptides are different.
   E.7.274. The immunogenic composition of any of E7.270-E7.273, wherein the first Pf bacteriophage peptide, or derivative, comprises or consists of SEQ ID NO: 3.
   E7.275. The immunogenic composition of any of E7.270-E7.274, wherein the second Pf bacteriophage peptide, or derivative, comprises or consists of SEQ ID NO: 21 or SEQ ID NO: 22.
   E7.276. The immunogenic composition of any of E7.271-E7.275, wherein the third Pf bacteriophage peptide, or derivative, comprises or consists of SEQ ID NO: 4.
   E7.277. An immunogenic composition comprising:
a first Pf bacteriophage peptide, or derivative, coupled to a first carrier; and
a second Pf bacteriophage peptide, or derivative, coupled to a second carrier;
wherein the first and second carriers may be the same or different;
wherein the first Pf bacteriophage peptide, or derivative, comprises or consists of any of SEQ ID NO: 20 to SEQ ID NO: 25; and
the second Pf bacteriophage peptide, or derivative, comprises or consists of any of the following options:
any of SEQ ID NO: 1 to SEQ ID NO: 2;
any of SEQ ID NO: 3 to SEQ ID NO: 9;
SEQ ID NO: 10;
any of SEQ ID NO: 11 to SEQ ID NO: 19;
SEQ ID NO: 26;
any of SEQ ID NO: 27 to SEQ ID NO: 31;
SEQ ID NO: 32; or
SEQ ID NO: 33.
   E7.278. An immunogenic composition comprising:
a first Pf bacteriophage peptide, or derivative, coupled to a first carrier; and
a second Pf bacteriophage peptide, or derivative, coupled to a second carrier;
wherein the first and second carriers may be the same or different;
wherein the first Pf bacteriophage peptide, or derivative, comprises or consists of SEQ ID NO: 10; and
the second Pf bacteriophage peptide, or derivative, comprises or consists of any of the following options:
any of SEQ ID NO: 1 to SEQ ID NO: 2;
any of SEQ ID NO: 3 to SEQ ID NO: 9;
any of SEQ ID NO: 11 to SEQ ID NO: 19;
any of SEQ ID NO: 20 to SEQ ID NO: 25;
SEQ ID NO: 26;
any of SEQ ID NO: 27 to SEQ ID NO: 31;
SEQ ID NO: 32; or
SEQ ID NO: 33.
   E7.279. An immunogenic composition comprising:
a first Pf bacteriophage peptide, or derivative, coupled to a first carrier; and
a second Pf bacteriophage peptide, or derivative, coupled to a second carrier;
wherein the first and second carriers may be the same or different;
wherein the first Pf bacteriophage peptide, or derivative, comprises or consists of any of SEQ ID NO: 1 to SEQ ID NO: 2; and
the second Pf bacteriophage peptide, or derivative, comprises or consists of any of the following options:
any of SEQ ID NO: 3 to SEQ ID NO: 9;
SEQ ID NO: 10;
any of SEQ ID NO: 11 to SEQ ID NO: 19;
any of SEQ ID NO: 20 to SEQ ID NO: 25;
SEQ ID NO: 26;
any of SEQ ID NO: 27 to SEQ ID NO: 31;
SEQ ID NO: 32; or
SEQ ID NO: 33.
   E7.280. An immunogenic composition comprising:
a first Pf bacteriophage peptide, or derivative, coupled to a first carrier; and
a second Pf bacteriophage peptide, or derivative, coupled to a second carrier;
wherein the first and second carriers may be the same or different;
wherein the first Pf bacteriophage peptide, or derivative, comprises or consists of any of SEQ ID NO: 27 to SEQ ID NO: 31; and
the second Pf bacteriophage peptide, or derivative, comprises or consists of any of the following options:
any of SEQ ID NO: 1 to SEQ ID NO: 2;
any of SEQ ID NO: 3 to SEQ ID NO: 9;
SEQ ID NO: 10;
any of SEQ ID NO: 11 to SEQ ID NO: 19;
any of SEQ ID NO: 20 to SEQ ID NO: 25;
SEQ ID NO: 26;
SEQ ID NO: 32; or
SEQ ID NO: 33.
   E7.281. An immunogenic composition comprising:
a first Pf bacteriophage peptide, or derivative, coupled to a first carrier; and
a second Pf bacteriophage peptide, or derivative, coupled to a second carrier;
wherein the first and second carriers may be the same or different;
wherein the first Pf bacteriophage peptide, or derivative, comprises or consists of SEQ ID NO: 26; and
the second Pf bacteriophage peptide, or derivative, comprises or consists of any of the following options:
any of SEQ ID NO: 1 to SEQ ID NO: 2;
any of SEQ ID NO: 3 to SEQ ID NO: 9;
SEQ ID NO: 10;
any of SEQ ID NO: 11 to SEQ ID NO: 19;
any of SEQ ID NO: 20 to SEQ ID NO: 25;
SEQ ID NO: 26;
any of SEQ ID NO: 27 to SEQ ID NO: 31;
SEQ ID NO: 32; or
SEQ ID NO: 33.
   E7.282. An immunogenic composition comprising:
a first Pf bacteriophage peptide, or derivative, coupled to a first carrier; and
a second Pf bacteriophage peptide, or derivative, coupled to a second carrier;
wherein the first and second carriers may be the same or different;
wherein the first Pf bacteriophage peptide, or derivative, comprises or consists of any of SEQ ID NO: 11 to SEQ ID NO: 19; and
the second Pf bacteriophage peptide, or derivative, comprises or consists of any of the following options:
any of SEQ ID NO: 1 to SEQ ID NO: 2;
any of SEQ ID NO: 3 to SEQ ID NO: 9;
SEQ ID NO: 10;
any of SEQ ID NO: 20 to SEQ ID NO: 25;
SEQ ID NO: 26;
any of SEQ ID NO: 27 to SEQ ID NO: 31;
SEQ ID NO: 32; or
SEQ ID NO: 33.
   E7.283. An immunogenic composition comprising:
a first Pf bacteriophage peptide, or derivative, coupled to a first carrier; and
a second Pf bacteriophage peptide, or derivative, coupled to a second carrier;
wherein the first and second carriers may be the same or different;
wherein the first Pf bacteriophage peptide, or derivative, comprises or consists of SEQ ID NO: 32; and
the second Pf bacteriophage peptide, or derivative, comprises or consists of any of the following options:
any of SEQ ID NO: 1 to SEQ ID NO: 2;
any of SEQ ID NO: 3 to SEQ ID NO: 9;
SEQ ID NO: 10;
any of SEQ ID NO: 11 to SEQ ID NO: 19;
any of SEQ ID NO: 20 to SEQ ID NO: 25;
SEQ ID NO: 26;
any of SEQ ID NO: 27 to SEQ ID NO: 31; or
SEQ ID NO: 33.
   E7.290. The immunogenic conjugate or immunogenic composition of any of E1-E7.283, wherein the Pf bacteriophage peptide derivative, at each occurrence, independently comprises, or consists of, a peptide having at least 80% identity with a Pf bacteriophage peptide selected from the group consisting of SEQ ID NO: 1 to SEQ ID NO: 33.
   E7.291. The immunogenic conjugate or immunogenic composition of any of E1-E7.283, wherein the Pf bacteriophage peptide derivative, at each occurrence, independently comprises, or consists of, a peptide having at least 85% identity with a Pf bacteriophage peptide selected from the group consisting of SEQ ID NO: 1 to SEQ ID NO: 33.
   E7.292. The immunogenic conjugate or immunogenic composition of any of E1-E7.283, wherein the Pf bacteriophage peptide derivative, at each occurrence, independently comprises, or consists of, a peptide having at least 90% identity with a Pf bacteriophage peptide selected from the group consisting of SEQ ID NO: 1 to SEQ ID NO: 33.
   E7.293. The immunogenic conjugate or immunogenic composition of any of E1-E7.283, wherein the Pf bacteriophage peptide derivative, at each occurrence, independently comprises, or consists of, a peptide having at least 94 or 95% identity with a Pf bacteriophage peptide selected from the group consisting of SEQ ID NO: 1 to SEQ ID NO: 33.
   E7.300. The immunogenic conjugate or immunogenic composition of any of E1-E7.293, wherein the Pf bacteriophage peptide, or derivative thereof attaches at the C-terminus.
   E8. The immunogenic conjugate or immunogenic composition of any of E1-E7.300, wherein each carrier is independently a carrier protein, virus-like particle (VLP), protein nanoparticle (NP), phage carrier, or other organic or inorganic particle suitable for generating an immune response against a hapten.
   E9. The immunogenic conjugate or immunogenic composition of E8, wherein each carrier is a carrier protein.
   E9.1. The immunogenic conjugate or immunogenic composition of E9, wherein the carrier protein is selected from the group consisting of human serum albumin, keyhole limpet hemocyanin, immunoglobulin molecules, thyroglobulin, ovalbumin, influenza hemagglutinin, PAN-DR binding peptide (PADRE polypeptide), malaria circumsporozite (CS) protein, hepatitis B surface antigen, Heat Shock Protein (HSP) 65, Bacillus Calmette-Guerin (BCG), cholera toxin, cholera toxin mutants with reduced toxicity, diphtheria toxin, CRM197 protein that is cross-reactive with diphtheria toxin, recombinant Streptococcal C5a peptidase, Streptococcus pyogenes ORF1224, Streptococcus pyogenes ORF1664, Streptococcus pyogenes ORF 2452, Chlamydia pneumoniae ORF T367, Chlamydia pneumoniae ORF T858, Tetanus toxoid, HIV gp120 T1, microbial surface components recognizing adhesive matrix molecules (MSCRAMMS), growth factor/hormone, cytokines and chemokines.
   E10. The immunogenic conjugate or immunogenic composition of E9, wherein the carrier protein is independently keyhole limpet hemocyanin (KLH), cross-reacting material (CRM) of diphtheria toxin, tetanus toxoid, meningococcal outer membrane protein complex, diphtheria toxoid, ExoProteinA (ExoA), or H. influenzae protein D.
   E10.1. The immunogenic conjugate or immunogenic composition of any of E9-E10, wherein the carrier protein is immunogenic.
   E10.2. The immunogenic conjugate or immunogenic composition of any of E9-E10.1, wherein the carrier protein is between about 20 kDa and 100 kDa.
   E10.3. The immunogenic conjugate or immunogenic composition of any of E9-E10.1, wherein the carrier protein is about 58 kDa.
   E10.4. The immunogenic conjugate or immunogenic composition of any of E9-E10.3, wherein the carrier protein is CRM197.
   E11. The immunogenic conjugate or immunogenic composition of any of E1-E10.4, wherein each Pf bacteriophage peptide, or derivative thereof, is coupled to the carrier by a linker.

The term “linker” refers to a divalent chemical group or combination of divalent chemical groups, where the divalent groups may comprise rings or chains. The divalent chemical group(s) are composed of atoms or combinations of atoms connected by covalent bonds. Linkers connect separate chemical moieties such as, for example, connecting a carrier binding moiety to a Pf bacteriophage peptide. The linker length may vary depending on the particular application. Generally, the linker contains at least a linear arrangement of from 2 to 50 atoms, through a combination of chain(s) and/or ring(s), where the linear arrangement may include side branching and/or substitution. The linkers may include one or more heteroatoms such as oxygen, nitrogen, sulfur, or phosphorus. Linkers may contain oxo groups, amino groups, alkyl groups, halogens and nitro groups. Linkers may also contain aryl groups. Linkers may comprise divalent moieties such as —C₁₋₁₂alkylene-, —(C₂₋₆alkylene-O)ₓ—C₁₋₆alkylene-, —C₁₋₆alkylene-, —C₂₋₆alkylene-O—, C₃₋₈cycloalkylene, —C(O)—, —O—, —S—, —S(O)—, —S(O)₂—, —NR²⁰—, —C(R²¹)═N—NH—, —CH(CO₂H)—,

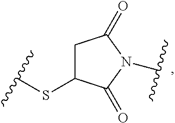 [SMILES: *SC1CC(=O)N(*)C1=O]

an amino acid moiety, a protected amino acid moiety, and phenylene; wherein the C₃₋₈cycloalkylene and phenylene are optionally independently substituted with 1-4 substituents independently selected from the group consisting of C₁₋₄alkyl, C₁₋₄haloalkyl, C₁₋₄alkoxy, halo, cyano, and hydroxy; R²⁰ and R²¹ at each occurrence are independently hydrogen or C₁₋₄alkyl; and x is an integer from 1 to 20.

Linkers may link to a carrier with divalent moieties such as

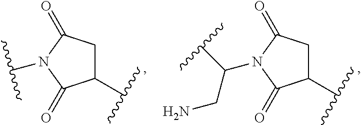 [SMILES: *C1CC(=O)N(*)C1=O ; *C1CC(=O)N(C(*)CN)C1=O]

—C(O)CH₂—, —NHC(S)—, —NHC(O)—, —C(O)—, —CH(SO₃H)—C(O)—,

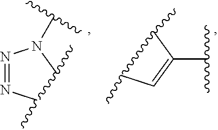

—NH—, or —N(C₁₋₆alkyl)-. The divalent moiety may be bonded to a carrier through a sulfhydryl group (e.g., cysteine), through an amino (e.g., lysine), through a carbonyl moiety (derived from a carboxyl), or through a ring formed by click chemistry (e.g., a triazole). For example, the moiety

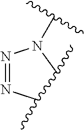

may bond to

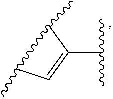

to form a triazole moiety

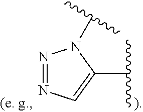 [SMILES: *c1cnnn1*]

The triazole is formed by [3+2] cycloaddition of an azide to an alkyne, where the azide may originate from a linker precursor and the alkyne from the carrier, or vice versa.

It is well known in the art that certain functional groups on proteins can facilitate conjugation of other molecules to the protein with cross-linking reagents. A variety of means of attaching the protein/peptide carriers to peptide immunogens are possible. Peptide immunogen-carrier conjugates have been successfully generated using various cross-linking reagents such as zero-length, homobifunctional or heterobifunctional cross linkers. The smallest available reagent systems for bioconjugation are the so-called zero-length cross-linkers. These compounds mediate the conjugation of two molecules by forming a bond containing no additional atoms. Homobifunctional reagents, which were the first cross-linking reagents used for modification and conjugation of macromolecules, consisted of bireactive compounds containing the same functional group at both ends. These reagents could tie one protein to another by covalently reacting with the same common groups on both molecules. Thus, the lysine amines or N-terminal amines of one protein could be cross-linked to the same functional groups on a second protein simply by mixing the two together in the presence of the homobifunctional reagent. Heterobifunctional conjugation reagents contain two different reactive groups that can couple to two different functional targets on proteins and other macromolecules. For example, one part of a cross-linker may contain an amine-reactive group, while another portion may consist of a sulfhydryl-reactive group. The result is the ability to direct the cross-linking reaction to selected parts of target molecules, thus garnering better control over the conjugation process. Heterobifunctional reagents are used to cross-link proteins and other molecules in a two- or three-step process that limits the degree of polymerization often obtained using homobifunctional cross-linkers.

Many methods are currently available for coupling of peptide immunogens to protein/polypeptide carriers using zero-length, homobifunctional or heterobifunctional crosslinkers. Most methods create amine, amide, urethane, isothiourea, or disulfide bonds, or in some cases thioethers. The more general method of coupling proteins or peptides to peptides utilizes bifunctional crosslinking reagents. These are small spacer molecules having active groups at each end. The spacer molecules can have identical or different active groups at each end. The most common active functionalities, coupling groups, and bonds formed are:
1. Aldehyde—amino→secondary amine
2. Maleimido—sulfhydryl→thioether
3. Succinimido—amino→amide
4. Imidate esters—amino→amide
5. Phenyl azides—amino→phenyl amine
6. Acyl halide—sulfhydryl→thioether
7. Pyridyldisulfides—sulfhydryl→disulfide
8. Isothiocyanate—amino→isothiourea.

Common homobifunctional linker reagents include:

Adipic acid dihydrazide
1,4-Bis[3-(2-pyridyldithio)propionamido]butane
4,4′-Diisothiocyanatostilbene-2,2′-disulfonic acid disodium salt hydrate
Dimethyl 3,3′-dithiopropionimidate dihydrochloride
Dimethyl pimelimidate dihydrochloride
3,3′-Dithiodipropionic acid di(N-hydroxysuccinimide ester)
Ethylene glycol-bis(succinic acid N-hydroxysuccinimide ester)
Sebacic acid bis(N-succinimidyl) ester
Suberic acid bis(N-hydroxysuccinimide ester)
Suberic acid bis(3-sulfo-N-hydroxysuccinimide ester) sodium salt

Common heterobifunctional linker reagents include:
Sulfosuccinimidyl 4(maleimidomethyl)cyclohexane-1-carboxylate (Sulfo-SMCC)
N-Succinimidyl 4-maleimidobutyrate (GMBS)
S-Acetylthioglycolic acid N-hydroxysuccinimide ester
5-Azido-2-nitrobenzoic acid N-hydroxysuccinimide ester
Bromoacetic acid N-hydroxysuccinimide ester
Maleimidoacetic acid N-hydroxysuccinimide ester
3-Maleimidobenzoic acid N-hydroxysuccinimide ester
4-(N-Maleimido)benzophenone
6-Maleimidohexanoic acid N-hydroxysuccinimide ester
4-(N-Maleimidomethyl)cyclohexanecarboxylic acid N-hydroxysuccinimide ester
4-(N-Maleimidomethyl)cyclohexane-1-carboxylic acid 3-sulfo-N-hydroxysuccinimide ester sodium salt
3-(2-Pyridyldithio)propionic acid N-hydroxysuccinimide ester

In some instances, optimal conjugation is more complex and may be advantageously accomplished through crosslinkers that are trifunctional, such as tris-(succinimidyl) aminotriacetate (TSAT), or otherwise multi or polyfunctional. Similarly, dendrimers such as (poly)amidoamine (PAMAM), may be employed to provide multifunctional conjugates.

The reactivity of a given carrier protein, in terms of its ability to be modified by a cross-linking agent such that it can be conjugated to a peptide immunogen, is determined by its amino acid composition and the sequence location of the individual amino acids in the three dimensional structure of the molecule, as well as by the amino acid composition of the peptide immunogen.

To facilitate the conjugation of a peptide immunogen with a carrier, additional amino acids can be added to the termini of the peptide. The additional residues can also be used for modifying the physical or chemical properties of the peptide immunogen. Amino acids such as tyrosine, cysteine, lysine, glutamic or aspartic acid, or the like, can be introduced at the C- or N-terminus of the peptide immunogen. Additionally, peptide linkers containing amino acids such as glycine and alanine can also be introduced. In addition, the antigenic determinants can differ from the natural sequence by being modified by terminal NH2-group acylation, e.g., by alkanoyl (C1-C20) or thioglycolyl acetylation, terminal-carboxy amidation, e.g., ammonia, methylamine, etc. In some instances, these modifications may provide sites for linking to a support or other molecule.

The peptide immunogen is linked to the protein/peptide carrier either directly or via a linker either at the amino or carboxy terminus of the peptide immunogen. The amino terminus of either the peptide immunogen or the protein/peptide carrier may be acylated.

Peptide immunogens can be linked to a carrier by chemical crosslinking. Techniques for linking an immunogen to a carrier include the formation of disulfide linkages using N-succinimidyl-3-(2-pyridyl-thio) propionate (SPDP) (Carlsson, J et al. (1978) Biochem J, 173: 723,) and succinimidyl 4-(N-maleimidomethyl) cyclohexane-1-carboxylate (SMCC) (if the peptide lacks a sulfhydryl group, this can be provided by addition of a cysteine residue to the hapten). These reagents create a disulfide linkage between themselves and peptide cysteine resides on one protein and an amide linkage through the amino on a lysine, or other free amino group in other amino acids. A variety of such disulfide/amide-forming agents are described in Immune. Rev. 62: 85 (1982). Other bifunctional coupling agents form a thioether rather than a disulfide linkage. The thioether forming agents include reactive ester of 6-maleimidocaproic acid, 2-bromoacetic acid, and 2-iodoacetic acid, 4-(N-maleimido-methyl) cyclohexane-1-carboxylic acid. The carboxyl groups can be activated by combining them with succinimide or 1-hydroxyl-2-nitro-4-sulfonic acid, sodium salt.

Most frequently, lysine residues are the most abundant amino acid residues found on carrier proteins, and these residues are modified using cross-linking reagents to generate nucleophilic sites that are then coupled to a hapten. This coupling is achieved via any of the hydrophilic side chains on the hapten molecules that are chemically active. These include the guanidyl group of arginine, the carboxyl groups of glutamate and aspartic acid, the sulfhydryl group of cysteine, and the amino group of lysine, to name a few. Modification of proteins such that they can now be coupled to other moieties is achieved using crosslinking reagents, which react with any of the side chains on the protein carrier or hapten molecules. In one aspect of the present invention, the carrier protein with or without a linker molecule is functionalized (derivatized) with a reagent that introduces reactive sites into the carrier protein molecule that are amenable to further modification to introduce nucleophilic groups. In one embodiment, the carrier is reacted with a haloacetylating reagent, which preferentially reacts with a number of functional groups on amino acid residues of proteins such as the sulfhydryl group of cysteine, the primary amine group of lysine residue, the a terminal of α-amines, the thioether of methionine and both imidazoyl side chain nitrogens of histidine (Gurd, 1967). The primary amine groups on lysine residues of the carrier protein may be derivatized with N-hydroxysuccinimidyl bromoacetate to generate a bromoacetylated carrier. Conjugation of peptide immunogen and the activated protein carrier is carried out by slowly adding the activated carrier to the solution containing the peptide immunogen.

Certain carriers have reducible interchain disulfides, i.e. cysteine bridges. Carriers may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into carriers through the reaction of lysines with 2-iminothiolane (Traut's reagent) resulting in conversion of an amine into a thiol.

In general, to prepare conjugates of a partially reduced carrier, the relevant carrier is reduced using a reducing agent such as dithiothreitol (DTT) or tricarbonyl ethylphosphine (TCEP) (about 1.8 equivalents) in PBS with 1 mM DTPA, adjusted to pH 8 with 50 mM borate. The solution is incubated at 37° C. for 1 hour, purified using a 50 mL G25 desalting column equilibrated in PBS/1 mM DTPA at 4° C. The thiol concentration, the protein concentration, and the ratio of thiol to carrier can be determined using procedures disclosed in U.S. Pat. No. 7,829,531.

Alternatively, free thiol groups may be introduced into a carrier through reaction of lysines of the carrier with 2-iminothiolane. Initially, the carrier to be conjugated may be buffer exchanged into 0.1 M phosphate buffer pH 8.0 containing 50 mM NaCl, 2 mM DTPA, pH 8.0 and concentrated to 5-10 mg/mL. Thiolation may be achieved through addition of 2-iminothiolane to the carrier.

Carriers contain functionalities suitable for derivatization with cross-linking reagents. Exemplary derivatized carrier functionalities include:

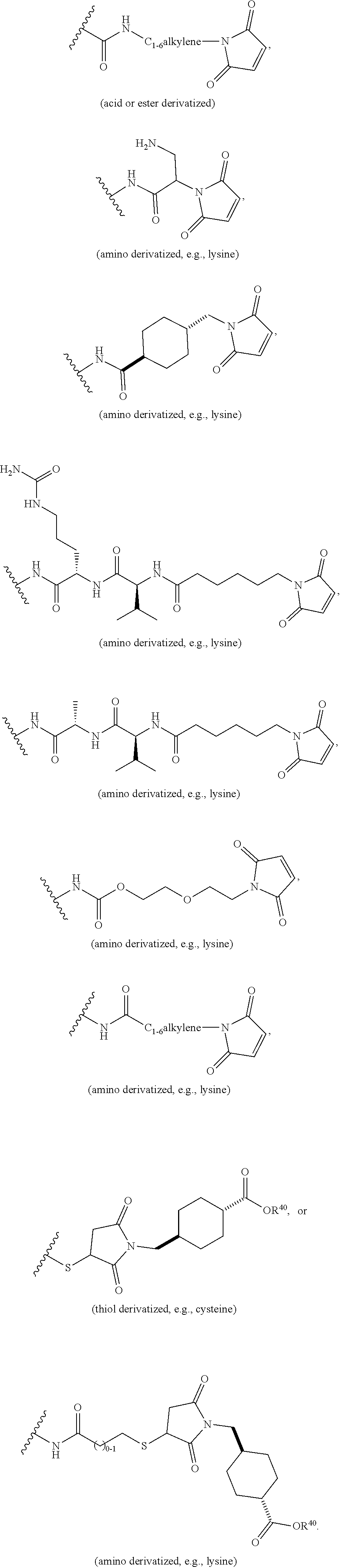

E12. The immunogenic conjugate or immunogenic composition of E11, wherein each linker consists of a linker moiety bonded to a linker-binding moiety, each linker-binding moiety also being bonded to a terminal amino acid of a Pf bacteriophage peptide, or derivative thereof.
E12.1. The immunogenic conjugate or immunogenic composition of E12, wherein at least one linker moiety is

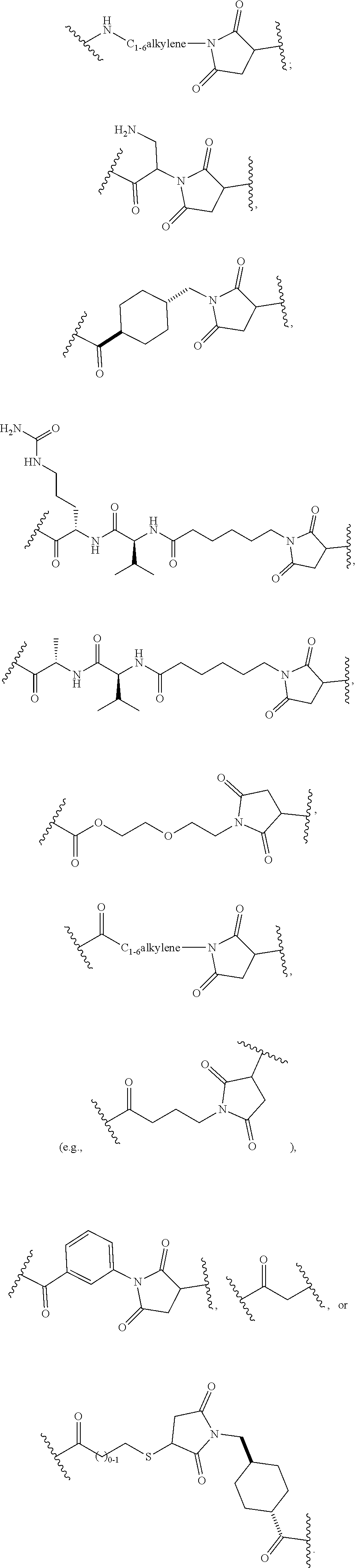

E12.2. Linker binding moieties of E12-12.1 include amino acids containing side or terminal functionality that is bonded to the terminus of the linker moiety by reaction of the amino acid functionality with a derivatized carrier. In practice, derivatized carriers may be further reacted with Pf bacteriophage peptides derivatized with linker-binding moiety functionalities (e.g., cysteine) to form the immunogenic conjugates of the invention, as illustrated below.

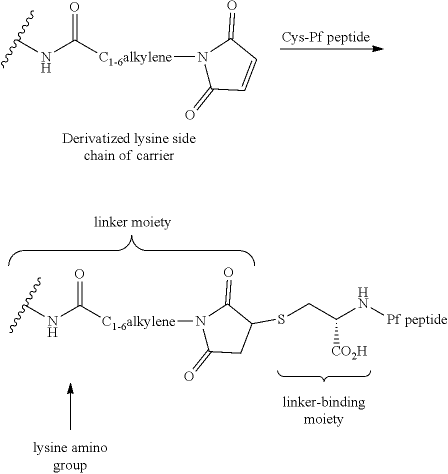

E13. The immunogenic conjugate or immunogenic composition of E12, wherein each linker moiety is a heterobifunctional linker moiety.
E14. The immunogenic conjugate or immunogenic composition of any of E11-E13, wherein each linker is bonded to a lysine of the carrier.
E15. The immunogenic conjugate or immunogenic composition of any of E12-E14, wherein each linker-binding moiety is a cysteine and the thiol of the cysteine is bonded to the linker moiety.
E15.1. The immunogenic conjugate or immunogenic composition of E15, wherein the linker having a cysteine linker-binding moiety is

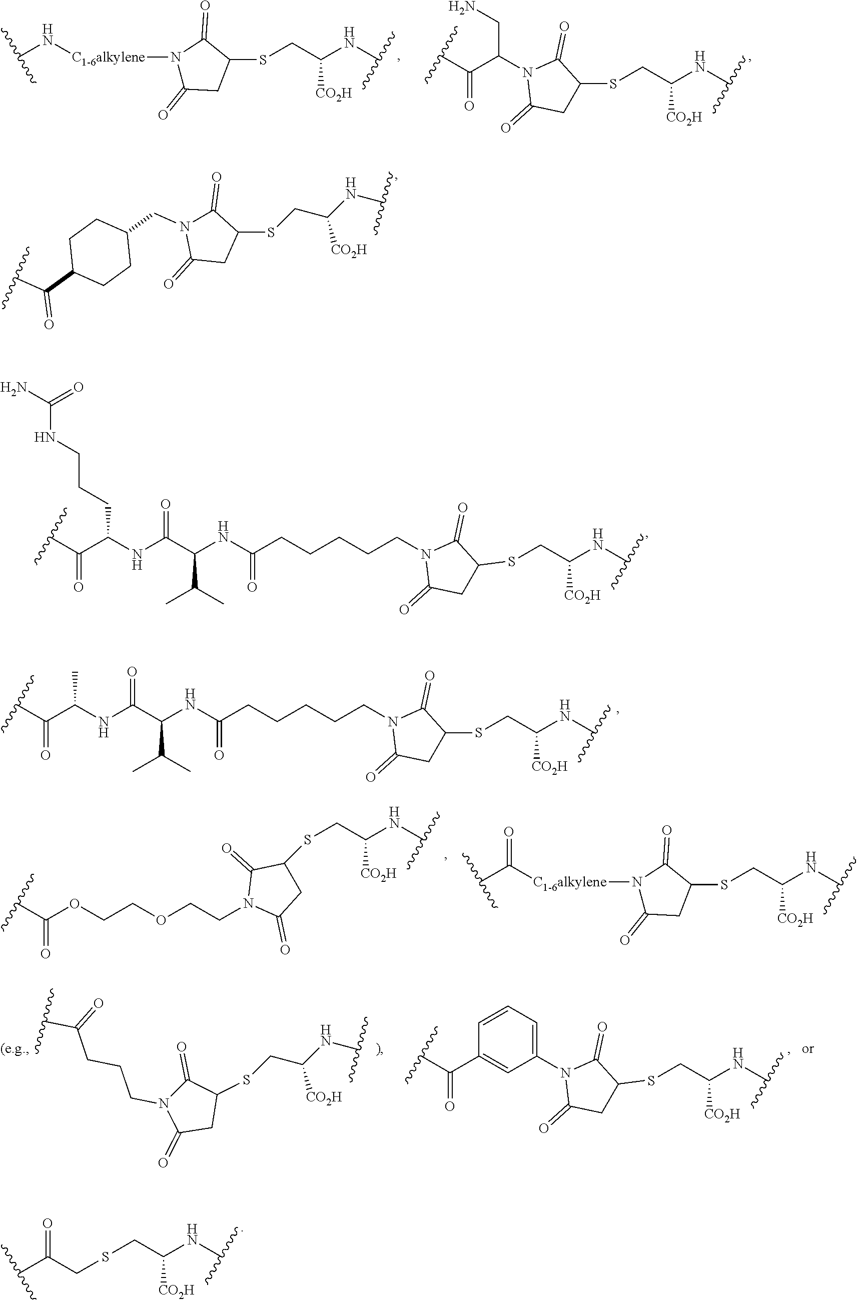

E16. The immunogenic conjugate or immunogenic composition of any of E11-E15, wherein at least one linker moiety is

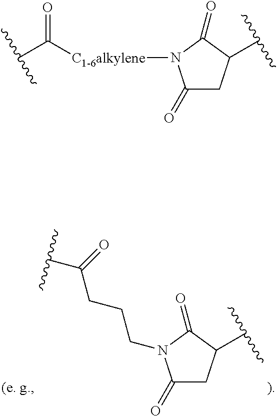

E17. The immunogenic conjugate or immunogenic composition of E16, wherein the at least one linker moiety bonds to the linker-binding moiety at the 3-position of the succinimide.
E18. An immunogenic composition comprising one or more than one different immunogenic conjugate of any of E1-E1.13 or E3-E17.
E18.1. The immunogenic composition of E18, comprising one immunogenic conjugate.
E18.2. The immunogenic composition of E18 comprising two or more different immunogenic conjugates.
E19. The immunogenic composition of any of E7.230-E7.293 or E18-E18.2, further comprising one or more adjuvants.
E20. A vaccine comprising the immunogenic conjugate or immunogenic composition of any of E1-E19.
E21a. A method of inducing or enhancing an immune response comprising administering to a subject in need thereof, an effective amount of the immunogenic conjugate of any of E1-E1.13 or E3-E17, the immunogenic composition of any of E7.230-E7.293 or E18-E18.2, or a vaccine thereof.
E21b. A method of inhibiting or reducing the capacity of a Pf bacteriophage to directly modulate an immune response (of a subject), the method comprising administering to a subject in need thereof, an effective amount of the immunogenic conjugate of any of E1-E1.13 or E3-E17, the immunogenic composition of any of E7.230-E7.293 or E18-E18.2, or a vaccine thereof.
E21c. A method of facilitating opsonization of Pa, the method comprising administering to a subject in need thereof, an effective amount of the immunogenic conjugate of any of E1-E1.13 or E3-E17, the immunogenic composition of any of E7.230-E7.293 or E18-E18.2, or a vaccine thereof.
E21d. A method of facilitating opsonization of Pa by binding virions associated with the Pa with an anti-Pf antibody, the method comprising administering to a subject in need thereof, an effective amount of the immunogenic conjugate of any of E1-E1.13 or E3-E17, the immunogenic composition of any of E7.230-E7.293 or E18-E18.2, or a vaccine thereof.
E21e. A method of binding virions associated with the Pa with an anti-Pf antibody, the method comprising administering to a subject in need thereof, an effective amount of the immunogenic conjugate of any of E1-E1.13 or E3-E17, the immunogenic composition of any of E7.230-E7.293 or E18-E18.2, or a vaccine thereof.
E21f. A method of interrupting or blocking Pf lifecycle associated with Pa, the method comprising administering to a subject in need thereof, an effective amount of the immunogenic conjugate of any of E1-E1.13 or E3-E17, the immunogenic composition of any of E7.230-E7.293 or E18-E18.2, or a vaccine thereof.
E22. The method of any of E21a-E21f, further comprising administering an effective amount of one or more adjuvants to the subject.
E23. A method of inducing or enhancing an immune response comprising administering to a subject in need thereof, an effective amount of the immunogenic composition of any of E2-E17 or E19, or a vaccine thereof.
E24. A method of inducing or enhancing an immune response comprising administering to a subject in need thereof,
   a) an effective amount of an immunogenic conjugate, or an immunogenic composition thereof, the immunogenic conjugate comprising a Pf bacteriophage peptide, or a derivative thereof, coupled to a carrier; and
   b) an effective amount of one or more adjuvants, the one or more adjuvants comprising a TLR4 agonist and/or a TLR7/8 agonist.
E24.1. The method of E24, wherein the immunogenic conjugate comprises a plurality of Pf bacteriophage peptides, or derivatives thereof, coupled to the carrier.
E24.2. The method of E24 or E24.1, wherein the Pf bacteriophage peptide, the coupling, and the carrier are as defined in any of E3-E17.
E24.3. A method of inducing or enhancing an immune response comprising administering to a subject in need thereof, an effective amount of the first and second, and optionally third immunogenic conjugate, or an immunogenic composition thereof, described in any of E7.230-E7.293.
E24.4. The method of E24.3, further comprising administering an effective amount of one or more adjuvants to the subject.
E25. The method of any of E21-E24.4, wherein the method reduces or prevents Pseudomonas aeruginosa biofilm formation in the subject.
E26. A kit comprising the immunogenic conjugate of any of E1-E17, the immunogenic composition of E18, or vaccine thereof, and one or more adjuvants.
E27. A kit comprising the immunogenic composition of any of E2-E17 or E19, or a vaccine thereof.
E28. The immunogenic composition, vaccine, method, or kit of any of E2-E17, E19-E20, or E22-E27, wherein the one or more adjuvants comprises a TLR4 agonist.
E28.1. The immunogenic composition, vaccine, method, or kit of any of E2-E17 or E28, wherein the TLR4 agonist is monophosphoryl lipid A (MPLA), glucopyranosyl lipid A (GLA), aminoalkyl glucosamide 4-phosphate (AGP), or Diamino allose phosphate (DAP), or a pharmaceutically acceptable salt thereof.
E28.2. The immunogenic composition, vaccine, method, or kit of any of E2-E17 or E28, wherein the TLR4 agonist is

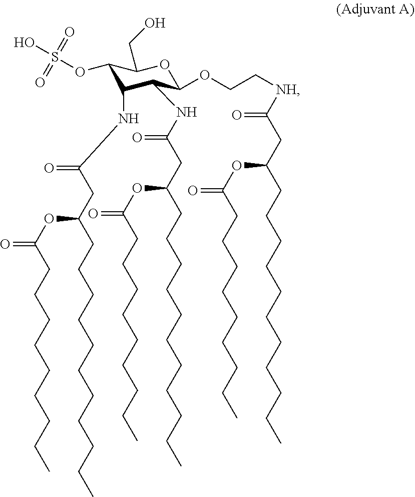

or a pharmaceutically acceptable salt thereof.
E29. The immunogenic composition, vaccine, method, or kit of any of E2-E17, E19-E20, or E22-E28.2, wherein the one or more adjuvants comprises a TLR7/8 agonist.
E29.1. The immunogenic composition, vaccine, method, or kit of any of E2-E17 or E29, wherein the TLR7/8 agonist is a compound of formula (I)

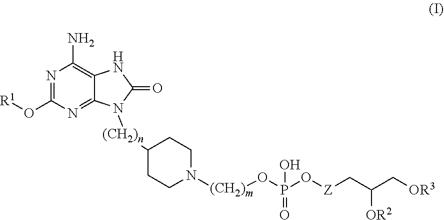

or a pharmaceutically acceptable salt thereof, wherein
R¹ is C₁-C₈ alkyl;
R² is H, C₆-C₂₀ alkyl, C₆-C₂₀ alkenyl, or C(O)R⁴;
R³ is C₆-C₂₀ alkyl, C₆-C₂₀ alkenyl, or C(O)R⁴;
R⁴, at each occurrence, is independently selected from C₆-C₂₀ alkyl and C₆-C₂₀ alkenyl;
n is 1, 2, 3, 4, 5, or 6;
m is 2, 3, 4, 5, or 6;
Z is (C₂-C₆ alkylene-O)_{q}; and
q is 1, 2, 3, 4, 5, 6, 7, 8, 9, 10, 11, 12, 13, 14, 15, 16, 17, 18, 19 or 20.
E29.2. The immunogenic composition, vaccine, method, or kit of E29.1, wherein R² is hydrogen or C(O)R⁴.
E29.3. The immunogenic composition, vaccine, method, or kit of any of E29.1 or E29.2, wherein R³ is C(O)R⁴.
E29.4. The immunogenic composition, vaccine, method, or kit of any of E29.1-E29.3, wherein R⁴, at each occurrence, is independently selected from (CH₂)₁₀CH₃, (CH₂)₁₂CH₃, (CH₂)₁₄CH₃, (CH₂)₁₆CH₃, and (CH₂)₇CH═CH(CH₂)₇CH₃.
E29.5. The immunogenic composition, vaccine, method, or kit of any of E29.1-E29.4, wherein R⁴ is (CH₂)₁₄CH₃.
E29.6. The immunogenic composition, vaccine, method, or kit of any of E29.1-E29.5, where n is 1.
E29.7. The immunogenic composition, vaccine, method, or kit of any of E29.1-E29.6, wherein m is 2.
E29.8. The immunogenic composition, vaccine, method, or kit of any of E29.1-E29.7, wherein Z is (C₂ alkylene-O)_{q}—.
E29.9. The immunogenic composition, vaccine, method, or kit of any of E29.1-E29.8, wherein q is 3, 6, 9, 12, or 16.
E29.10. The immunogenic composition, vaccine, method, or kit of any of E29.1-E29.9, wherein q is 3.
E29.11. The immunogenic composition, vaccine, method, or kit of E29.1, wherein the compound of formula (I) is a compound of formula (Ia):

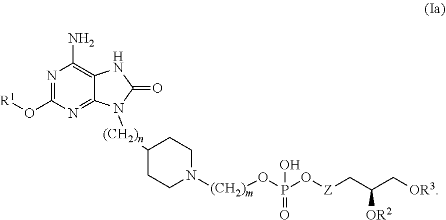

E29.12. The immunogenic composition, vaccine, method, or kit of E29.1, wherein the compound of formula (I) is selected from the group consisting of:

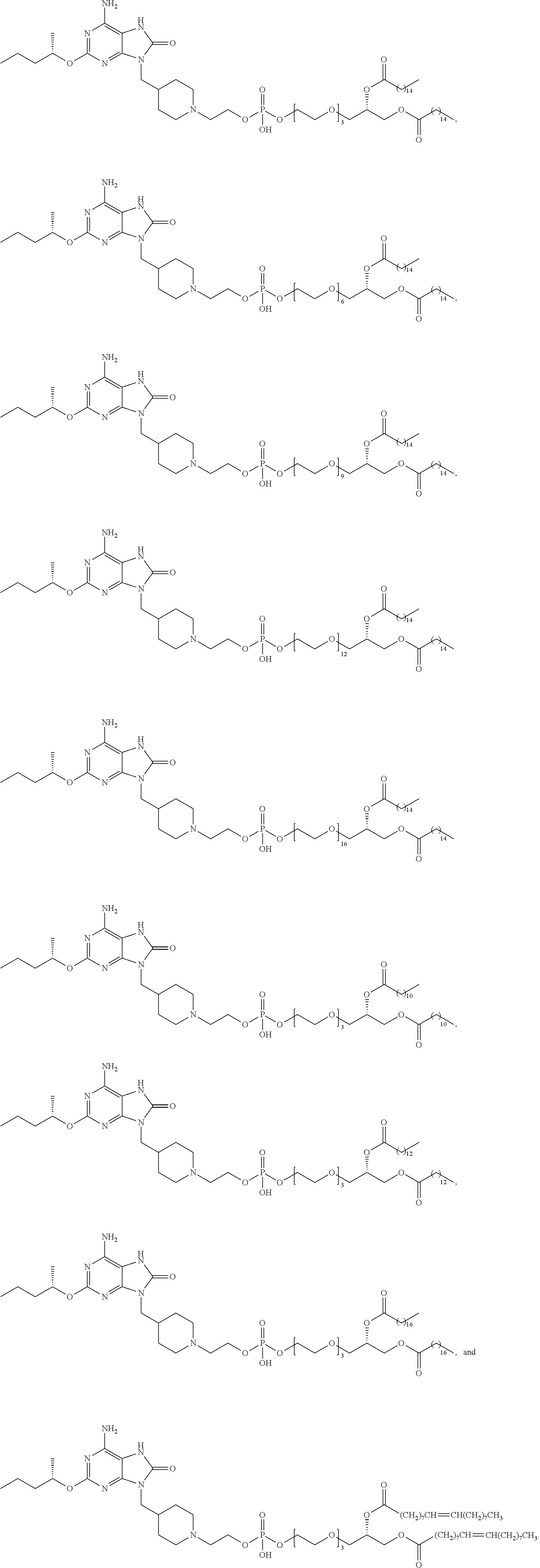

E29.13. The immunogenic composition, vaccine, method, or kit of E29.1, wherein the compound of formula (I) is

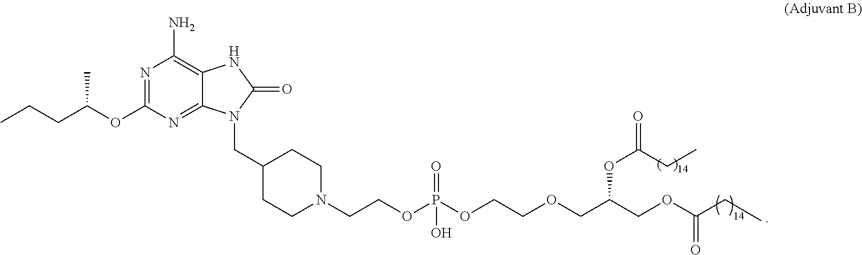

E29.14. The immunogenic composition, vaccine, method, or kit of any one of E29.1-E29.13, wherein the pharmaceutically acceptable salt is choline salt.
E29.15. The immunogenic composition, vaccine, method, or kit of any of E2-E17 or E29, wherein the TLR7/8 agonist is oxoadenine or an imidazoquinoline, such as resiquimod or imiquimod.
E30. The immunogenic composition, vaccine, method, or kit of any of E2-E17, E19-E20, or E22-E29.15, wherein the one or more adjuvants further comprises alum, saponin or a derivative thereof, QS-21, TQL1055, and/or isotucaresol.

### 3. Adjuvants

The immunogenic compositions and vaccines may include an adjuvant. Adjuvants are additives that enhance humoral and/or cell mediated immune responses to a vaccine antigen. Any adjuvant may be useful in the pharmaceutical compositions and formulations described herein. The adjuvant may interact with a member or members of the TLR family. The adjuvant may be a TLR4 or TLR7/8 ligand.

TLR4 ligands include CRX-601, monophosphoryl lipid A (MPLA), glucopyranosyl lipid A (GLA), CRX-547,

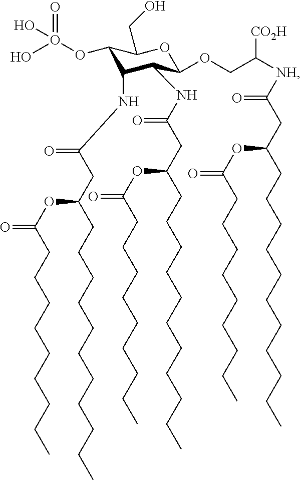

aminoalkyl glucosamide 4-phosphate (AGP) class lipid A mimetics, and other TLR4 ligands described in Khalaf et al., Bioorg. Med Chem. Lett. (2015) 25(3), 547-553, and U.S. Pat. Nos. 7,960,522 and 7,063,967, which are incorporated herein by reference. Other TLR4 ligands include Diamnio-allose phosphate compounds described in WO2019/157509, which is incorporated herein by reference. For example, the TLR4 ligand may be

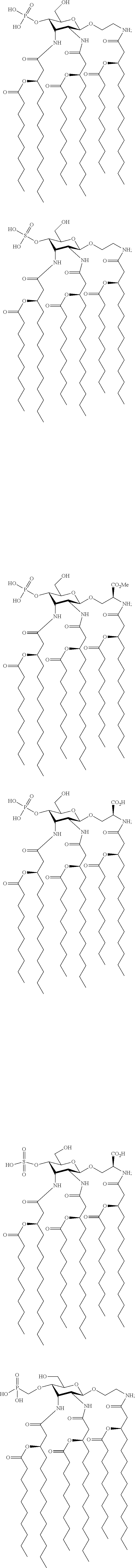

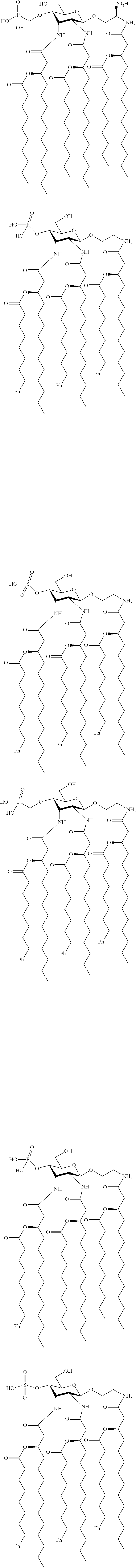

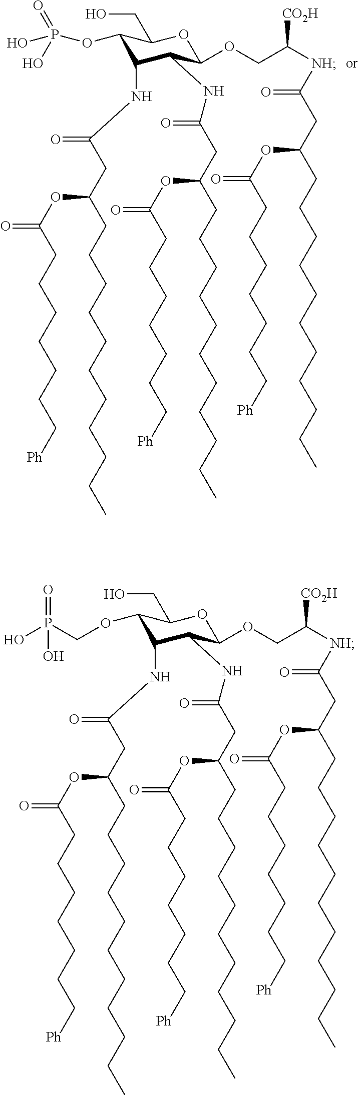

or a pharmaceutically acceptable salt thereof.

TLR7/8 ligands include oxoadenine or an imidazoquinoline, such as resiquimod or imiquimod. Other TLR7/8 ligands include compounds of formula (I).

Suitable adjuvants are, for example, saponin or a derivative thereof, such as QS-21 or TQL1055 (as described in WO2019/079160, which is incorporated herein by reference), isotucaresol, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham); mineral salts (for example, aluminum, silica, kaolin, and carbon); aluminum salts such as aluminum hydroxide gel (alum), AlK(SO₄)₂, AlNa(SO₄)₂, AlNH₄(SO₄), and Al(OH)₃; salts of calcium (e.g., Ca₃(PO₄)₂), iron or zinc; an insoluble suspension of acylated tyrosine; acylated sugars; cationically or anionically derivatized polysaccharides; polynucleotides (for example, poly IC and poly AU acids); polyphosphazenes; cyanoacrylates; polymerase-(DL-lactide-co-glycoside); biodegradable microspheres; liposomes; lipid A and its derivatives; monophosphoryl lipid A; wax D from Mycobacterium tuberculosis, as well as substances found in Corynebacterium parvum, Bordetella pertussis, and members of the genus Brucella); bovine serum albumin; diphtheria toxoid; tetanus toxoid; edestin; keyhole-limpet hemocyanin; Pseudomonal Toxin A; choleragenoid; cholera toxin; pertussis toxin; viral proteins; and Quil A. Aminoalkyl glucosamine phosphate compounds can also be used (see, e.g., WO 98/50399, U.S. Pat. No. 6,113,918 (which issued from U.S. Ser. No. 08/853,826), and U.S. Ser. No. 09/074,720). In addition, adjuvants such as cytokines (e.g., GM-CSF or interleukin-2, -7, or -12), interferons, or tumor necrosis factor, may also be used as adjuvants. Protein and polypeptide adjuvants may be obtained from natural or recombinant sources according to methods well known to those skilled in the art. When obtained from recombinant sources, the adjuvant may comprise a protein fragment comprising at least the immunostimulatory portion of the molecule. Other known immunostimulatory macromolecules which can be used include, but are not limited to, polysaccharides, tRNA, non-metabolizable synthetic polymers such as polyvinylamine, polymethacrylic acid, polyvinylpyrrolidone, mixed polycondensates (with relatively high molecular weight) of 4′,4-diaminodiphenylmethane-3,3′-dicarboxylic acid and 4-nitro-2-aminobenzoic acid (See, Sela, M., Science 166: 1365-1374 (1969)) or glycolipids, lipids or carbohydrates.

In some embodiments, the adjuvant is an aluminum salt. The aluminum salt may include phosphate, sulfate, hydroxide, or a combination thereof. In some embodiments, the aluminum salt is potassium aluminum sulphate, which may be in hydrated form. The alumimun salt may also be an aluminum hydroxide gel. The alumimun salt may also be an aluminium phosphate wet gel. Aluminum salt adjuvants may be referred to as alum. In some embodiments, a compound as disclosed herein is adsorbed to the aluminum salt.

“Saponin,” as the term is used herein, encompasses natural and synthetic glycosidic triterpenoid compounds and pharmaceutically acceptable salts, derivatives, mimetics (e.g., isotucaresol and its deriviatives) and/or biologically active fragments thereof, which possess immune adjuvant activity.

In one illustrative embodiment, saponins employed in the vaccine compositions of the present invention can be purified from Quillaja saponaria Molina bark, as described in U.S. Pat. No. 5,057,540, the disclosure of which is incorporated herein by reference in its entirety.

The adjuvant properties of saponins were first recognized in France in the 1930's. (see, Bomford et al., Vaccine 1992, 10: 572-577). Two decades later the saponin from the bark of the Quillaja saponaria Molina tree found wide application in veterinary medicine, but the variability and toxicity of these crude preparations precluded their use in human vaccines. (see, Kensil et al., In Vaccine Design: The Subunit and Adjuvant Approach; Powell, M. F., Newman, J. J., Eds.; Plenum Press: New York, 1995 pp. 525-541).

In the 1970's a partially purified saponin fraction known as Quil A was shown to give reduced local reactions and increased potency (see, Kensil et al., 1995). Further fractionation of Quil A, which consisted of at least 24 compounds by HPLC, demonstrated that the four most prevalent saponins, QS-7, QS-17, QS-18, and QS-21, were potent adjuvants (see, Kensil, C. R. Crit Rev. Ther. Drug Carrier Syst. 1996, 13, 1-55; Kensil et al., 1995). QS-21 and QS-7 were the least toxic of these. Partly because of its reduced toxicity, highly purified state (though still a mixture of no less than four compounds), (see, Soltysik, S.; Bedore, D. A.; Kensil, C. R. Ann. N.Y. Acad. Sci. 1993, 690: 392-395) and more complete structural characterization, QS-21 (3) was the first saponin selected to enter human clinical trials. (see, Kensil, 1996; Kensil et al., 1995).

QS-21 and other Quillaja saponins increase specific immune responses to both soluble T dependent and T-independent antigens, promoting an Ig subclass switch in B-cells from predominantly IgG1 or IgM to the IgG2a and IgG2b subclasses (Kensil et al., 1995). The IgG2a and IgG2b isotypes are thought to be involved in antibody dependent cellular cytotoxicity and complement fixation (Snapper and Finkelman, In Fundamental Immunology, 4th ed.; Paul, W. E., Ed.: Lippincott-Raven: Philadelphia, Pa., 1999, pp. 831-861). These antibody isotypes also correlate with a Th-1 type response and the induction of IL-2 and IFN-γ-cytokines which play a role in CTL differentiation and maturation (Constant and Bottomly, Annu. Rev. Immunology 1997, 15: 297-322). As a result, QS-21 and other Quillaja saponins are potent inducers of class I MHC-restricted CD8+ CTLs to subunit antigens (Kensil, 1996; Kensil et al., 1995).

According to an aspect of the present invention, a saponin employed in the immunostimulant composition comprises a Quillaja saponin. In one preferred embodiment of this aspect of the invention, the Quillaja saponin comprises QS-7, QS-17, QS-18 and/or QS-21.

According to another aspect of the present invention, a saponin employed in the immunostimulant composition comprises a triterpene saponin-lipophile conjugate comprising a nonacylated or desacylated triterpene saponin that includes a 3-glucuronic acid residue; and a lipophilic moiety; wherein said saponin and said lipophilic moiety are covalently attached to one another, either directly or through a linker group, and wherein said direct attachment or attachment to said linker occurs through a covalent bond between the carboxyl carbon of said 3-glucuronic acid residue, and a suitable functional group on the lipophilic residue or linker group.

The triterpene saponin can have a triterpene aglycone core structure with branched sugar chains attached to positions 3 and 28, and an aldehyde group linked or attached to position 4; and is either originally non-acylated, or require removal of an acyl or acyloyl group that is bound to a saccharide at the 28-position of the triterpene aglycone. The triterpene saponin can have a quillaic acid or gypsogenin core structure. Some saponin-lipophile conjugates useful in this invention, including GPI-0100, a quilaja saponin-lipophile conjugate, are disclosed in U.S. Pat. Nos. 5,977,081 and 6,080,725, each of which is incorporated herein by reference in its entirety. The desacylsaponin or nonacylated saponin can be selected from the group consisting of Quillaja desacylsaponin, S. jenisseensis desacylsaponin, Gypsophila saponin, Saponaria saponin, Acanthophyllum saponin and lucyoside P saponin.

The lipophilic moiety can comprise one or more residues of a fatty acid, terpenoid, aliphatic amine, aliphatic alcohol, aliphatic mercapto mono- or poly-C 2-C4 alkyleneoxy derivative of a fatty acid, mono- or poly-C2-C4 alkyleneoxy derivative of a fatty alcohol, glycosyl-fatty acid, glycolipid, phospholipid or a mono-, or di-acylglycerol.

In another aspect of the present invention, the saponin employed in the immunostimulant composition comprises a saponin/antigen covalent conjugate composition.

QS-21 and other Quillaja saponins can be purified from Quillaja saponaria using standard biochemical methodologies. Briefly, aqueous extracts of Quillaja saponaria Molina bark are dialyzed against water. The dialyzed extract is lyophilized to dryness, extracted with methanol, and the methanol-soluble extract is further fractionated on silica gel chromatography and by reverse phase high pressure liquid chromatography (RP-HPLC). The individual saponins are then be separated by reverse phase HPLC. At least 22 peaks (denominated QA-1 to QA-22, also referred to herein as QS-1 to QS-21) are separable using this approach, with each peak corresponding to a carbohydrate peak and exhibiting a single band on reverse phase thin layer chromatography. The individual components can be specifically identified by their retention times on a C4 HPLC column, for example.

Preferably, the Quillaja saponins employed according to this embodiment of the invention correspond to peaks QS-7, QS-17, QS-18, and/or QS-21, as described in U.S. Pat. No. 5,057,540. In one specific embodiment of the invention, QS-21 saponin is used in accordance with this disclosure.

The substantially pure QS-7 saponin is characterized as having immune adjuvant activity and containing about 35% carbohydrate (as assayed by anthrone) per dry weight. QS-7 has a UV absorption maxima of 205-210 nm, a retention time of approximately 9-10 minutes on RP-HPLC on a Vydac C 4 column having 5 m particle size, 330 angstrom pore, 4.6 mm ID×25 cm L in a solvent of 40 mM acetic acid in methanol/water (58/42; v/v) at a flow rate of 1 ml/min, eluting with 52-53% methanol from a Vydac C4 column having 5 m particle size, 330 angstrom pore, 10 mM ID×25 cm L in a solvent of 40 mM acetic acid with gradient elution from 50 to 80% methanol, having a critical micellar concentration of approximately 0.06% in water and 0.07% in phosphate buffered saline, causing no detectable hemolysis of sheep red blood cells at concentrations of 200 μg/ml or less, and containing the monosaccharide residues terminal rhamnose, terminal xylose, terminal glucose, terminal galactose, 3-xylose, 3,4-rhamnose, 2,3-fucose, and 2,3-glucuronic acid, and apiose.

The substantially pure QS-17 saponin is characterized as having adjuvant activity and containing about 29% carbohydrate (as assayed by anthrone) per dry weight. QS-17 has a UV absorption maxima of 205-210 nm, a retention time of approximately 35 minutes on RP-HPLC on a Vydac C 4 column having 5 m particle size, 330 angstrom pore, 4.6 mm ID×25 cm L in a solvent of 40 mM acetic acid in methanol-water (58/42; v/v) at a flow rate of 1 ml/min, eluting with 63-64% methanol from a Vydac C4 column having 5 m particle size, 330 angstrom pore, 10 mm ID×25 cm L in a solvent of 40 mM acetic acid with gradient elution from 50 to 80% methanol, having a critical micellar concentration of 0.06% (w/v) in water and 0.03% (w/v) in phosphate buffered saline, causing hemolysis of sheep red blood cells at 25 μg/ml or greater, and containing the monosaccharide residues terminal rhamnose, terminal xylose, 2-fucose, 3-xylose, 3,4-rhamnose, 2,3-glucuronic acid, terminal glucose, 2-arabinose, terminal galactose and apiose.

The substantially pure QS-18 saponin is characterized as having immune adjuvant activity and containing about 25-26% carbohydrate (as assayed by anthrone) per dry weight. QS-18 has a UV absorption maxima of 205-210 nm, a retention time of approximately 38 minutes on RP-HPLC on a Vydac C 4 column having 5 m particle size, 330 angstrom pore, 4.6 mm ID×25 cm L in a solvent of 40 mM acetic acid in methanol/water (58/42; v/v) at a flow rate of 1 ml/min, eluting with 64-65% methanol from a Vydac C4 column having 5 m particle size, 330 angstrom pore, 10 mm ID×25 cm L in a solvent of 40 mM acetic acid with gradient elution from 50 to 80% methanol, having a critical micellar concentration of 0.04% (w/v) in water and 0.02% (w/v) in phosphate buffered saline, causing hemolysis of sheep red blood cells at concentrations of 25 g/ml or greater, and containing the monosaccharides terminal rhamnose, terminal arabinose, terminal apiose, terminal xylose, terminal glucose, terminal galactose, 2-fucose, 3-xylose, 3,4-rhamnose, and 2,3-glucuronic acid.

The substantially pure QS-21 saponin is characterized as having immune adjuvant activity and containing about 22% carbohydrate (as assayed by anthrone) per dry weight. The QS-21 has a UV absorption maxima of 205-210 nm, a retention time of approximately 51 minutes on RP-HPLC on a Vydac C 4 column having 5 m particle size, 330 angstrom pore, 4.6 mm ID×25 cm L in a solvent of 40 mM acetic acid in methanol/water (58/42; v/v) at a flow rate of 1 ml/min, eluting with 69 to 70% methanol from a Vydac C4 column having 5 m particle size, 330 angstrom pore, 10 mm ID×25 cm L in a solvent of 40 mM acetic acid with gradient elution from 50 to 80% methanol, with a critical micellar concentration of about 0.03% (w/v) in water and 0.02% (w/v) in phosphate buffered saline, causing hemolysis of sheep red blood cells at concentrations of 25 μg/ml or greater, and containing the monosaccharides terminal rhamnose, terminal arabinose, terminal apiose, terminal xylose, 4-rhamnose, terminal glucose, terminal galactose, 2-fucose, 3-xylose, 3,4-rhamnose, and 2,3-glucuronic acid.

### 4. Uses and Methods

When a foreign antigen challenges the immune system, it responds by launching a protective response that is characterized by the coordinated interaction of both the innate and acquired immune systems. These two interdependent systems fulfill two mutually exclusive requirements: speed (contributed by the innate system) and specificity (contributed by the adaptive system).

The innate immune system serves as the first line of defense against invading pathogens, holding the pathogen in check while the adaptive responses are matured. It is triggered within minutes of infection in an antigen-independent fashion, responding to broadly conserved patterns in the pathogens (though it is not non-specific, and can distinguish between self and pathogens). Crucially, it also generates the inflammatory and co-stimulatory milieu (sometimes referred to as the danger signal) that potentiates the adaptive immune system and steers (or polarizes it) towards the cellular or humoral responses most appropriate for combating the infectious agent. The development of TLR modulators for therapeutic targeting of innate immunity has been reviewed (see Nature Medicine, 2007, 13, 552-559; Drug Discovery Today: Therapeutic Strategies, 2006, 3, 343-352 and Journal of Immunology, 2005, 174, 1259-1268).

The adaptive response becomes effective over days or weeks, but ultimately provides the fine antigenic specificity required for complete elimination of the pathogen and the generation of immunologic memory. It is mediated principally by T and B cells that have undergone germline gene rearrangement and are characterized by specificity and long lasting memory. However, it also involves the recruitment of elements of the innate immune system, including professional phagocytes (macrophages, neutrophils etc.) and granulocytes (basophils, eosinophils etc.) that engulf bacteria and even relatively large protozoal parasites. Once an adaptive immune response has matured, subsequent exposure to the pathogen results in its rapid elimination due to highly specific memory cells have been generated that are rapidly activated upon subsequent exposure to their cognate antigen.

In certain embodiments, the compounds, conjugates and compositions provided herein elicit a cell mediated immune and/or a humoral immune response. In other embodiments, the immune response induces long lasting (e.g. neutralizing) antibodies and a cell mediated immunity that quickly responds upon exposure to the infectious agent.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognize or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response, a TH2 immune response and a TH17 response.

A TH1 immune response may include one or more of an increase in CTLs, an increase in one or more of the cytokines associated with a TH1 immune response (such as IL-2, IFN-γ, and TNF-0), an increase in activated macrophages, an increase in NK activity, or an increase in the production of IgG2a. Preferably, the enhanced TH1 immune response will include an increase in IgG2a production.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 and IgE production.

A Th17 immune response may include one or more of an increase in one or more of the cytokines associated with a TH17 immune response (such as IL-17, IL-22, IL-23, TGF-beta and IL-6), or an increase in humoral immunity and memory B cells.

In certain embodiments, the immune response is one or more of a TH1 immune response, a TH2 response and a TH17 response. In other embodiments, the immune response provides for an enhanced TH1 response, TH2 response, and/or TH17 response.

In certain embodiments, the enhanced immune response is one or both of a systemic and a mucosal immune response. In other embodiments, the immune response provides for one or both of an enhanced systemic and an enhanced mucosal immune response. In certain embodiments, the mucosal immune response is a TH1, TH2, or TH17 immune response. In certain embodiments, the mucosal immune response includes an increase in the production of lgA.

In certain embodiments the immunogenic compositions provided herein are used as vaccines, wherein such compositions include an immunologically effective amount of one or more antigens.

Compounds, conjugates, and compositions provided herein may be useful for eliciting or enhancing or modifying or suppressing in a host at least one immune response (e.g., a TH1-type T lymphocyte response, a TH2-type T lymphocyte response, a TH17-type T lymphocyte response, a cytotoxic T lymphocyte (CTL) response, an antibody response, a cytokine response, a lymphokine response, a chemokine response, and an inflammatory response). In certain embodiments the immune response may comprise at least production of one or a plurality of cytokines wherein the cytokine is selected from interferon-gamma (IFN-7), tumor necrosis factor-alpha (TNF-α), production of one or a plurality of interleukins wherein the interleukin is selected from IL-1, IL-2, IL-3, IL-4, IL-6, IL-8, IL-10, IL-12, IL-13, IL-16, IL-18 and IL-23, production one or a plurality of chemokines wherein the chemokine is selected from MIP-1α, MIP-1β, RANTES, IP-10, CCL4 and CCL5, and a lymphocyte response that is selected from a memory T cell response, a memory B cell response, an effector T cell response, a cytotoxic T cell response and an effector B cell response.

Pf bacteriophage are temperate phages that infect the bacterium Pseudomonas aeruginosa, (Pa) a pathogen associated with infections in wounds, burns, and nosocomial settings. There are at least seven Pf phages that have been identified including Pf1, Pf2, Pf3, Pf4 Pf5, Pf6 and Pf7. Interestingly, despite being a pathogen itself, Pf can “act as a virulence factor for P. aeruginosa and promote the pathogenesis of Pa infections in animal models. The presence of Pf in Pa is associated with worse skin and lung infections in humans” P. R. Secor, Front. Immuno. 11:244 February 2020 p1. and is shown to have significant effects on biofilm formation, antibiotic resistance, and motility, including the suppression of mammalian immunity at sites of bacterial infection. Moreover, Pf phage are now strongly implicated in promoting chronic Pa infections, such as those associated with cystic fibrosis. These chronic infections pose an enormous financial and clinical burden on the health care system.

Pf virions are composed of thousands of copies of a single major coat protein (CoaB in Pf) arranged in an elongated, helical symmetry, with minor coat proteins at either end and encasing a single-stranded circular DNA genome. Adsorption of Pf virion into the Pa periplasm is mediated by the minor coat protein CoaA on the end of the virion. Once the Pf phage has infected the cell, depending on various levels of host stress, the phage may initiate a chronic infection and produce progeny virions or the phage may initiate a lysogenic lifecycle and integrate into the bacterial chromosome as a prophage.

The fundamental structure of the virion is similar among the various strains of Pf phage. Generally, Pf phage CoaB protein is approximately 8.7 kD between and 40 and 55 amino acids in length. Following the cleaving of the leader sequence within the bacterial periplasm, there are three primary domains in the CoaB protein: an acidic domain, a hydrophobic domain and a basic domain. Due to its overall negative charge the acidic domain of the CoaB coat protein is exposed to environment, while the neutrally charged hydrophobic domain remains buried within phage virion structure. The basic domain, which is positively charged, interacts with negatively charged phage DNA inside the capsid. Thus, the exterior of the Pf phage coat is composed almost exclusively of the “exposed,” acidic regions of the CoaB proteins.

While the Pf phage CoaB proteins share some general characteristics, differences in the sequences of the coat protein among Pf phage variants can be found. The Pf phage variants can be organized into phylogenetic clades, based upon the differences in the amino acid sequence of the variant's CoaB protein, using commercially available software, for example ClustalW using default parameters (Pairwise Alignment: Alignment Speed=Slow, Open Gap Penalty=15, and Extend Gap Penalty=6.66; Multiple Alignment: Open Gap Penalty=15, and Extend Gap Penalty=6.66; Delay Divergent=30%, and Transitions=Weighted), as described by Thompson et al. in Current Protocols in Bioinformatics 2003, vol 00 (1) pp. 2.3.1-2.3.22 (doi.org/10.1002/0471250953.bi0203s00). software. By focusing the clade analysis on the “exposed” acidic epitope of the CoaB protein phylogenic clades of the acidic epitope of the coat protein can be discerned.

The Pf bacteriophage peptides of the conjugate can be of any length, including the full length of the CoaB coat protein. The peptide should be long enough to provide sufficient specificity for binding but not so long as to adversely impact the immunogenic composition, for example, by being too insoluble for formulation or promoting undesirable peptide folding. Preferably the Pf bacteriophage peptides are between 5 to 45 amino acids in length, or between 7-36, or between 12-25, or 14-22 amino acids in length.

The Pf bacteriophage peptide has a net charge at physiological pH (about 7.4). In some embodiments a Pf bacteriophage peptide with a formal net charge is advantageous, for example, for purposes of formulation. In some embodiments the net charge is a positive or is a negative charge. In some embodiments the net charge of the peptide is one of −3, −2 or −1, preferably −1 or −2. In some embodiments the net charge of the peptide is +1, +2 or +3, preferably +1 or +2 Net charge can be calculated using standard techniques known to those of ordinary skill in the art.

Disclosed herein is an immunogenic composition described for use in treating or preventing Pa infection in a patient in need thereof.

The present disclosure also includes die use of the immunogenic composition, as herein described, in the manufacture of a medicament for treating or preventing a Pa infection in a patient in need thereof.

The present disclosure also includes the method comprising the step of administering to a patient an effective amount of the immunogenic composition as herein described.

Persons skilled in the art will be familiar with the type of infections that are associated with Pa, including but not limited to infection associated with a wound, infection associated with a burn, nosocomial infection (including Pa infection associated with patient cauterization and patient ventilation and surgery), infection associated with radiation and chemotherapy cancer treatments, as well as with chronic Pa infection, such as is associated with cystic fibrosis patients and patients with AIDS

In a particular embodiment, the immunogenic conjugates are used to treat or prevent diseases and conditions associated with or mediated by Pf bacteriophage such as, for example, to treat subjects infected with or at risk of being infected with Pseudomonas aeruginosa. Accordingly, in one embodiment the immunogenic composition described herein is administered to a subject with a wound or a burn. In another embodiment the immunogenic composition described herein is administered to a subject at risk of a nosocomial-derived Pa infection, can be used to prevent, treat or slow the progression Pa in a subject In another embodiment the immunogenic composition described herein is administered to a subject diagnosed with or at risk of having cystic fibrosis. In another embodiment the immunogenic composition described herein is administered to a subject diagnosed with or at risk of having cancer. In another embodiment the immunogenic composition described herein is administered to a subject requiring radiation or chemotherapy. In another embodiment the immunogenic composition described herein is administered to a subject diagnosed with or at risk of HIV infection or AIDS. In another embodiment the immunogenic composition described herein is administered to a subject diagnosed with or at risk of diabetes.

Compounds, conjugates, and compositions provided herein may be useful for effecting in a host at least one of the following: inhibiting or reducing the capacity of a Pf bacteriophage to directly modulate an immune response of a host, as may be indicated by increased clearance of Pa from the host; facilitating opsonization of Pa as may be indicated by virions associated with the Pa being bound with an anti-Pf antibody; or interrupting or blocking the Pf lifecycle associated with Pa as may be indicated by decreased Pf viral load in a host.

### 5. Formulations and Modes of Administration

In another aspect of the invention, pharmaceutically acceptable compositions are provided, wherein these compositions comprise any of the compounds or conjugates as described herein, and optionally comprise a pharmaceutically acceptable carrier, adjuvant or vehicle. These compositions optionally further comprise one or more additional therapeutic agents.

In an aspect of the present invention, pharmaceutical compositions may be manufactured by processes well known in the art, e.g., by means of conventional mixing, dissolving, granulating, dragee-making, levigating, emulsifying, encapsulating, entrapping or lyophilizing processes.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl (e.g., phenyl/substituted phenyl) sulfonate.

As described herein, the pharmaceutically acceptable compositions of the invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds or conjugates of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The pharmaceutically acceptable compositions of this invention can be administered to humans and other animals orally, rectally, parenterally, intracistemally, intradermally, intranasally, intravaginally, intraperitoneally, intramuscularly, intravenously, intratumorally, topically (as by powders, ointments, or drops), bucally, sublingually, as an oral or nasal spray, or the like, depending on the severity of the disease being treated.

Preferred routes of administration for the disclosed immunogenic compositions include, for example, injection (e.g., subcutaneous, intramuscular, intravenous, parenteral, intraperitoneal, intrathecal). The injection can be in a bolus or a continuous infusion. Other preferred routes of administration include oral administration.

Pharmaceutical compositions for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions can also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It can also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle.

Liquid dosage forms for oral or nasal administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, cement, putty, thin film, and granules. In such solid dosage forms, the active compound can be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form can also comprise buffering agents.

Solid compositions of a similar type may also be employed as fillers in soft and hardfilled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or trans dermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In preferred embodiments, the compounds or conjugates of the described invention can be formulated either as pharmaceutically acceptable salts or free acids. Compounds may be formulated with a pharmaceutically acceptable vehicle for injection, inhalation, ingestion or other suitable form of administration. A pharmaceutically acceptable vehicle is a medium, solution or matrix that does not interfere with the immunomodulatory activity of the compound or conjugate and is not toxic to the patient and preferably lends significant physical and chemical stability to the API. Pharmaceutically acceptable vehicles include aqueous solution, liposomes, oil-in-water or water-in-oil emulsions, polymeric particles, block-copolymers, aqueous dispersions, microparticles, proteins solutions or biodegradable particles for timed release. For example, the vehicle may be a microsphere, nanoparticle or microparticle having a compound or conjugate of this invention in the matrix of the particle or adsorbed on the surface. The vehicle may also be an aqueous solution, buffered solution or micellar dispersion containing monoethanol amine, triethylamine, triethanolamine, or other chemical that renders the formulation alkaline. The vehicle may be a suspension containing aluminum hydroxide, aluminum phosphate, calcium hydroxide, or calcium phosphate where the compound or conjugate may be adsorbed to the metal surface. Vehicles may also include all solvents, buffers, dispersion media, vehicles, coatings, diluents, antibacterial and antifungal agents, mucoadhesives, mucopenetrants, absorption delaying agents, packing agents, suspensions, colloids, and the like. The use of such vehicles for APIs is well known to those skilled in the art. Except vehicles or agents which are incompatible with the API, their use in prophylactic or therapeutic compositions is considered.

In one embodiment compounds of the invention are formulated in 2% glycerol or 2% glycine as an isotonic nanodispersion with a pH in the range of 5 to 7.4. In another embodiment the compounds of the invention are formulated in the lipid bilayer of a liposome. These liposomes may also contain other compounds with immunomodulatory activity to achieve a co-formulation with the compounds of the invention. More generally the compounds of the invention may be encapsulated in a nano or microparticle, emulsion, or other suitable vehicle as described above and these may also contain other immunomodulatory compounds or excipients to enhance biological activity, improve stability or alter pharmacokinetics of the formulation in a favorable way.

The phrase “therapeutically effective amount” of the present compounds means sufficient amounts of the compounds to treat disorders, at a reasonable benefit/risk ratio applicable to any medical treatment. It is understood, however, that the total daily dosage of the compounds and compositions can be decided by the attending physician within the scope of sound medical judgment. The specific therapeutically effective dose level for any particular patient can depend upon a variety of factors including the disorder being treated and the severity of the disorder; activity of the specific compound employed; the specific composition employed; the age, body weight, general health and prior medical history, sex and diet of the patient; the time of administration, route of administration, and rate of excretion of the specific compound employed; the duration of the treatment; drugs used in combination or coincidental with the specific compound employed; and like factors well-known in the medical arts. For example, it is well within the skill of the art to start doses of the compound at levels lower than required to achieve the desired therapeutic effect and to gradually increase the dosage until the desired effect is achieved. Actual dosage levels of active ingredients in the pharmaceutical compositions can be varied to obtain an amount of the active compound(s) that is effective to achieve the desired therapeutic response for a particular patient and a particular mode of administration.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The adjuvant and vaccine compositions may include an “effective amount” of the disclosed compound or conjugate. In the context of an adjuvant or vaccine composition, an “effective amount” refers to an amount effective, at dosages and for periods of time necessary, to achieve the desired result (e.g., to potentiate an immune response to one or more antigens). The immune response can be measured, for example, by measuring antibody titers against an antigen, assessing the ability of a vaccine containing the compound to immunize a host in response to a disease or antigen challenge, etc. For example, administering an “effective amount” of a compound or composition to a subject increases one or more antibody titers by 10% or more over a nonimmune control, by 20% or more over a nonimmune control, by 30% or more over a nonimmune control, by 40% or more over a nonimmune control, by 50% or more over a nonimmune control, by 50% or more over a nonimmune control, by 70% or more over a nonimmune control, by 80% or more over a nonimmune control, by 90% or more over a nonimmune control, or by 100% or more over a nonimmune control.

Vaccine preparation is a well-developed art and general guidance in the preparation and formulation of vaccines is readily available from any of a variety of sources. One such example is New Trends and Developments in Vaccines, edited by Voller et al., University Park Press, Baltimore, Md., U.S.A. 1978.

Multiple administrations of the vaccine or immunogenic composition, including booster administrations may in some instances provide increased effectiveness. The optimal dosing regimen and conditions can be determined using standard procedures and techniques known to persons skilled in the art. For example, the compositions may be administered 1, 2, 3 or more times and at different periodic intervals.

In the treatment of certain medical conditions, repeated or chronic administration of compounds can be required to achieve the desired therapeutic response. “Repeated or chronic administration” refers to the administration of compounds daily (i.e., every day) or intermittently (i.e., not every day) over a period of days, weeks, months, or longer.

For adults, the doses are generally from about 0.00001 to about 100 mg/kg, desirably about 0.0001 to about 100 mg/kg body weight per day by inhalation, intranasal, intratumoral, sublingual, intradermal, or intrperitoneal, from about 0.00001 to about 100 mg/kg, desirably 0.0001 to 70 mg/kg, more desirably 0.5 to 10 mg/kg body weight per day by oral administration, and from about 0.00001 to about 50 mg/kg, desirably 0.0001 to 1 mg/kg body weight per day by intravenous administration.

Combination therapy includes administration of a single pharmaceutical dosage formulation containing one or more of the compounds and conjugates described herein and one or more additional pharmaceutical agents, as well as administration of the compounds, conjugates, and each additional pharmaceutical agent, in its own separate pharmaceutical dosage formulation. For example, a compound or conjugate described herein and one or more additional pharmaceutical agents, can be administered to the patient together, in a single oral dosage composition having a fixed ratio of each active ingredient, such as a tablet, capsule, or injectable; or each agent can be administered in separate oral dosage formulations. Where separate dosage formulations are used, the present compounds, conjugates and one or more additional pharmaceutical agents can be administered at essentially the same time (e.g., concurrently) or at separately staggered times (e.g., sequentially).

In the methods and uses described herein, the pharmaceutical combination of a conjugate and adjuvant, or a pharmaceutically acceptable salt, or composition thereof; and/or additional therapeutic agent may be administered/used simultaneously, separately, or sequentially, and in any order, and the components may be administered separately or as a fixed combination. For example, treatment according to the invention may comprise administration of a first active ingredient in free or pharmaceutically acceptable salt form and administration of a second active ingredient in free or pharmaceutically acceptable salt form, simultaneously or sequentially in any order, in jointly therapeutically effective amounts or effective amounts, e.g. in daily dosages corresponding to the amounts described herein. The individual active ingredients of the combination can be administered separately at different times during the course of therapy or concurrently in divided or single dosage forms. The instant invention is therefore to be understood as embracing all such regimes of simultaneous or alternating treatment and the term “administering” is to be interpreted accordingly. Thus, a pharmaceutical combination, as used herein, defines either a fixed combination in one dosage unit form or separate dosages forms for the combined administration where the combined administration may be independently at the same time or at different times. As a further example, conjugates and adjuvants or therapeutic agents may be administered/used simultaneously (e.g., through coinjection), or separately, or sequentially, such as administration of the conjugate followed by administration of the adjuvant or therapeutic agent, or vice versa.

The pharmaceutical compositions and formulations may include additional therapeutic agents. In some embodiments, the additional therapeutic agent is an adjuvant, an immunostimulant, an antiviral, an antibiotic, a therapeutic antibody, another vaccine, a chemotherapeutic agent, or a combination thereof.

The disclosed compounds may be included in kits comprising the compound, conjugate, adjuvant, or a pharmaceutically acceptable salt, a pharmaceutical composition, or both; and information, instructions, or both that use of the kit will provide treatment for medical conditions in mammals (particularly humans). The information and instructions may be in the form of words, pictures, or both, and the like. In addition or in the alternative, the kit may include the medicament, a composition, or both; and information, instructions, or both, regarding methods of application of medicament, or of composition, preferably with the benefit of treating or preventing medical conditions in mammals (e.g., humans).

The kits may contain one or more containers containing an additional therapeutic agent, including but not limited to those listed above. In certain embodiments, the kits may contain one or more containers containing an antigen(s), as described herein. In some embodiments the kits may be provided in the form of a vaccine composition as described herein, and optionally includes a syringe for injecting a subject with the vaccine composition.

### 6. Examples

### Example 1

### Preparation of Conjugates

Peptides of CoaB protein of filamentous Pf-family bacteriophage, with a sequence length of between 18 and 20 amino acids were used to covalently modify CRM-197 protein to form a monovalent conjugate protein (only one type/variant of peptide was conjugated to the protein) or a multivalent conjugate protein (two or more types/variants of peptides were conjugated to the protein).

The peptide was covalently linked to the carrier protein CRM-197 using N-γ-maleimidobutyryl-oxysuccinimide ester (GMBS). GMBS is a heterobifunctional crosslinker which modifies CRM-197 by reacting to the amine residues of the amino acid lysine at neutral pH. The resulting modified amino acid features then a new functional site that specifically reacts to the thiol residue of the amino acid cysteine of the peptide at neutral pH. There are 39 lysine residues in the sequence of CRM-197, however due to steric hindrance, not all residues are accessible to the crosslinker.

To promote successful protein modification, CRM-197 was diluted from a starting concentration of 2.1 mg/mL to 1.5 mg/mL using 0.2M NaPB pH=7.2, in order to maintain a neutral pH throughout the activation reaction with GMBS.

To activate CRM-197, GMBS was dissolved in DMSO to a concentration of 25 mg/mL. In order to more efficiently attach 10 copies of peptide to the protein, 15.6-17 copies of GMBS were first attached to CRM-197 by adding 85 equivalents of GMBS/DMSO solution to CRM-197 and allowing a conjugation time of 30 minutes at 25° C. The conjugation reaction was conducted in a temperature regulated water bath in the absence of any form of agitation.

Following the activation of CRM-197, the unreacted GMBS crosslinker was removed by ultrafiltration using 30 kDa Amicon Ultra centrifugal filters to allow the removal of residual GMBS through the filter membrane. The retained crosslinker-protein retentate was then washed 4× using fresh 0.2M sodium phosphate buffer (NaPB) pH=7.2 in order to minimize residual GMBS. Following purification of the activated CRM-197, a protein concentration of 1.5 mg/mL was adjusted, a theoretical concentration can be assumed, since protein losses during purification are negligible.

Table 1 shows a summary of mono-, di- and trivalent peptide-CRM-197 protein conjugates, that were prepared using bioconjugation chemistry. The average peptide copy #resembles the number of peptides that was attached to each molecule of protein CRM-197. The conjugate concentration is the average protein concentration determined by BCA assay.

TABLE 1

| | | | | Concen- |
|---|---|---|---|---|
| | Peptide | | Peptide | tration |
| | sequence | Type | copy# | (μg/mL) |
| Conjugate | GVIDTSAVESAITDG | monovalent | 10.75 | 2004.1 |
| A | QGDMC | | | |
| | (SEQ ID NO: 36) | | | |
| Conjugate | ESLLDETTKGVLAQA | monovalent | 10.13 | 1797.3 |
| B | STDGC | | | |
| | (SEQ ID NO: 37) | | | |
| Conjugate | GVIDTSAVEAAITEG | monovalent | 10.52 | 1716.8 |
| C | KGDMC | | | |
| | (SEQ ID NO: 38) | | | |
| Conjugate | SEQ ID NO: 36/ | divalent | 10.52 | 1675.4 |
| D | SEQ ID NO: 37 | | | |
| Conjugate | SEQ ID NO: 36/ | trivalent | 10.99 | 1856.6 |
| E | SEQ ID NO: 36/ | | | |
| | SEQ ID NO: 37 | | | |
| Conjugate | SEQ ID NO: 36/ | trivalent | 10.95 | 1747.3 |
| F | SEQ ID NO: 37/ | | | |
| | SEQ ID NO: 38 | | | |

In order to determine the level of GMBS modification, the molecular weight of the purified activated CRM was determined by MALDI TOF. The level of modification of CRM-197 with GMBS was determined using the difference of the molecular weight of CRM-197 and activated CRM. Purified activated CRM was frozen at −30° C. for later use.

In order to enable a facile reaction with the activated CRM-197, 40 molar equivalents of peptide were prepared for reaction. To prepare the monovalent peptide-CRM conjugate, the 1.5 mg/mL solution of activated CRM in 0.2M NaPB pH=7.2 was added directly to dry peptide. To prepare the multivalent peptide-CRM conjugate, an equimolar solution of peptides was prepared first in 0.2M NaPB pH=7.2, before activated CRM was added to the mixed peptide solution. The relative excess of peptide was held constant at 40 molar equivalents.

The activated CRM solution was added to the peptide and a reaction time of 2 hours was allowed at 25° C. Ultrafiltration was used to remove unreacted peptide from the peptide-CRM conjugate, utilizing 30 kDa Amicon Ultra centrifugal filters. The washing buffer and the reconstitution buffer was 10 mM PB at a pH of 7.2.

Following the purification of the final peptide-CRM conjugate, the protein solution was sterile filtered using a 0.22 μm PVDF syringe filter.

MALDI TOF MS was used to determine the molecular weight of the final peptide-CRM product. The difference between the molecular weight of activated CRM and peptide-CRM was used to derive the level of peptide addition to the protein.

Table 2 shows a summary of molecular weights determined by MALDI TOF. The weights were determined at all stages of peptide to CRM-197 bioconjugation, using GMBS as heterobifunctional crosslinker. First, the molecular weight of CRM-197, followed by the weight of purified activated CRM-197. The difference in molecular weights can be used to determine the average number of GMBS modification on the protein. Third, the molecular weight of purified peptide-CRM-197 conjugate. The difference to activated CRM-197 protein can be used to determine the average copy number of peptide added.

TABLE 2

| | MW (MALDI) | Difference | GMBS copy# | Peptide copy# |
|---|---|---|---|---|
| CRM | 58189.41 | | | |
| activated CRM | 60829.00 | 2639.59 | 15.81 | |
| Conjugate A | 81989.29 | 21160.29 | | 10.75 |
| Conjugate B | 81474.91 | 20645.91 | | 10.13 |
| Conjugate C | 81520.11 | 20691.11 | | 10.52 |
| Conjugate D | 81915.12 | 21086.12 | | 10.52 |
| CRM | 58040.11 | | | |
| activated CRM | 60918.31 | 2878.20 | 17.23 | |
| Conjugate D | 82797.01 | 21878.70 | | 10.99 |
| Conjugate E | 82.718.34 | 21800.03 | | 10.95 |

As expected, steric hindrance prevented some of the otherwise available functional sites on the activated CRM protein to be accessed and modified by peptide during the conjugation reaction. Substantially all the remaining, unreacted viable functional sites of activated CRM were hydrolyzed.

### Example 2

### Immune Activation by Conjugate A

CoaB peptide with the sequence GVIDTSAVESAITDGQGDMC (SEQ ID NO: 36) was conjugated to the carrier protein CRM-197 (Conjugate A) with a copy number of 9.9 was formulated and in some instances adjuvanted with the synthetic TLR4 adjuvant A in a 2.5% glycine formulation, as shown below.

Study design/Timeline: Female C57/BL/6 mice ages 6-8 weeks old were vaccinated with the noted antigen and adjuvant dose as listed in the study design below on days 0 and 14. Peripheral blood was collected 14 days following the first vaccination by submandibular bleeds. Five days post-second vaccination, spleens were collected from 5 out of 10 mice and processed for cell-mediated immunity assays. On day 28 (14 days post-second) blood was collected via cardiac puncture on the remaining 5 mice.

TABLE 3
Anti-Peptide A ELISA^{a,b}

| | Con- | | Adjuvant | | | | | | | | | |
|---|---|---|---|---|---|---|---|---|---|---|---|---|
| Group | jugate | | A | | Total IgG | | IgG1 | | IgG2b | | IgG2c | |
| # | A Dose | | Dose | | Mean | SD | Mean | SD | Mean | SD | Mean | SD |
| 1 | 0 | | 0 | | 0 | 0 | 0 | 0 | 0 | 0 | 0 | 0 |
| 2 | 1 | μg | 0.0001 | μg | 1.27 | 1.10 | 0.95 | 0.57 | 1.02 | 1.04 | 0.82 | 0.64 |
| 3 | 1 | μg | 0.001 | μg | 2.38 | 0.87 | 1.86 | 1.16 | 1.22 | 1.07 | 0 | 0 |
| 4 | 1 | μg | 0.01 | μg | 3.15 | 0.51 | 1.99 | 0.53 | 2.45 | 1.15 | 1.67 | 1.00 |
| 5 | 1 | μg | 0.1 | μg | 4.00* | 0.70 | 2.45 | 1.66 | 3.90** | 0.80 | 1.49 | 1.22 |
| 6 | 1 | μg | 1 | μg | 4.20*** | 0.51 | 3.60* | 0.51 | 4.31* | 0.41 | 3.37**** | 0.82 |
| 7 | 1 | μg | 10 | μg | 4.25** | 0.65 | 2.88* | 1.28 | 3.31 | 1.45 | 3.51**** | 0.72 |

^{a}Peptide A is GVIDTSAVESAITDGQGDM (SEQ ID NO: 3)
^{b}A mean value of 0 indicates that no anti-peptide specific antibody was detected with a serum dilution of 1:20.

Table 3 shows anti-peptide A serum antibody titers at 14 days post second and standard deviations from groups vaccinated with noted doses of Adjuvant A and Conjugate A. Asterisks denote statistical significance versus the antigen alone controls (Group 2, antigen+0.0001 ug adjuvant). Ordinary one-way ANOVA followed by Fisher's LSD were used to determine statistical analysis where *=p≤0.05, **=p≤0.01, ***=p≤0.001, ****=p≤0.0001.

All mice except from the naïve group received both 1 μg Conjugate A and various doses of Adjuvant A. The total IgG antibody response increased in an Adjuvant A dose-dependent manner with the highest observed titers at doses of 0.1 μg, 1 μg, 10 μg plus 1 μg antigen. Total IgG antibody responses also appear to plateau after the 0.1 μg dose of Adjuvant A. Based on this data the optimal dose of Adjuvant A may be 0.1 μg or 1 μg.

Table 3 also shows serum IgG1(A), IgG2b(B) and IgG2c (C) antibody titers from groups vaccinated with noted doses of Adjuvant A and 1 μg Conjugate A. Asterisks denote statistical significance versus the angien+0.0001 μg adjuvant group (Group 2). Ordinary one-way ANOVA followed by Fisher's LSD were used to determine statistical analysis where *=p≤0.05, **=p≤0.01, ***=p≤0.001, ****=p≤0.0001

Mice receiving 0.1 μg Adjuvant A had similar anti-Peptide A total IgG antibody titers as those groups receiving 1 μg and 10 μg of Adjuvant A. However, 0.1 μg Adjuvant A plus Conjugate A elicited lower anti-CoaB IgG1 and IgG2c antibody titers compared to groups receiving 1 μg or 10 μg of Adjuvant A, although these differences were not statistically significant. IgG1 antibody titers decreased in the group receiving 10 μg of Adjuvant A compared to group receiving 1 μg and the same pattern is exhibited for IgG2b antibody titers. Mice that received 1 μg of Adjuvant A had the highest IgG1 and IgG2b antibody response. Based on this data, 1 μg of Adjuvant A in combination with 1 μg of Conjugate A is an optimal dose for high anti-CoaB antibody titers of all isotypes.

### Example 3

### Immune Activation by Conjugate A

In this study, Conjugate A with a copy number of 10.7 was used. In some instances, synthetic TLR4 Adjuvant A in a 2.5% glycine was added and dose was selected based on data from previous studies. In other instances, Alydrogel® alum adjuvant was added. Doses were established by previous studies where a fixed antigen/alum ratio of 1:1.5 was optimal

Female C57/BL/6 mice ages 6-8 weeks old were vaccinated with the noted antigen and adjuvant dose as listed in the study design below on days 0, 14, and 28. Peripheral blood was collected 14 days following the first vaccination (14 days post-first “14dp1”) by submandibular bleeds. On day 28 (14 days post-second “14dp2”) peripheral blood was collected once more following the second vaccination by submandibular bleeds. On day 42 (14 days post-third “14dp3”), blood was collected via cardiac puncture on all mice, and spleens as well as lymph nodes were collected.

TABLE 4a
Anti-Peptide A ELISA^{a, b}

| | | | | Total IgG | |
|---|---|---|---|---|---|
| Group | Conjugate A | | | 14dp1 | |
| # | Dose | Adjuvant A Dose | Alum Dose | Mean | SD |
| 1 | 0 | | | 0 | 0 |
| 2 | 0.1 μg | | | 0 | 0 |
| 3 | 1 μg | | | 0.80 | 0.73 |
| 4 | 10 μg | | | 1.07 | 0.92 |
| 5 | 0.1 μg | | 1.65 μg | 0 | 0 |
| 6 | 1 μg | | 3 μg | 2.51^{c} | 0.76 |
| 7 | 10 μg | | 16.5 μg | 4.00^{c} | 0.17 |
| 8 | 0.1 μg | 1 μg | | 3.50^{c} | 0.56 |
| 9 | 1 μg | 1 μg | | 4.38^{c} | 0.33 |
| 10 | 10 μg | 1 μg | | 4.49^{c} | 0.43 |
| 11 | 0.1 μg | 1 μg | 1.65 μg | 2.46^{c} | 1.13 |
| 12 | 1 μg | 1 μg | 3 μg | 4.14^{c} | 0.36 |
| 13 | 10 μg | 1 μg | 16.5 μg | 4.27^{c} | 0.21 |

TABLE 4b
Anti-Peptide A ELISA^{a,b}

| | | | | | | Total | | Total | | | | | |
|---|---|---|---|---|---|---|---|---|---|---|---|---|---|
| | Con- | | | | | IgG | | IgG | | IgG1 | | IgG2b | |
| Group | jugate | | Adjuvant | Alum | | 14dp2 | | 14dp3 | | 14dp3 | | 14dp3 | |
| # | A Dose | | A Dose | Dose | | Mean | SD | Mean | SD | Mean | SD | Mean | SD |
| 1 | 0 | | | | | 0 | 0 | 0.68 | 0.44 | 0 | 0 | 0.82 | 0.46 |
| 2 | 0.1 | μg | | | | 0 | 0 | 2.10 | 2.13 | 1.62 | 1.80 | 1.42 | 1.07 |
| 3 | 1 | μg | | | | 1.82 | 0.82 | 3.29 | 1.57 | 2.95 | 1.42 | 2.19 | 1.03 |
| 4 | 10 | μg | | | | 1.38 | 0.87 | 3.51 | 0.95 | 3.09 | 1.04 | 1.62 | 0.89 |
| 5 | 0.1 | μg | | 1.65 | μg | 1.07 | 0.53 | 3.31 | 0.53 | 2.52 | 0.82 | 1.87 | 0.54 |
| 6 | 1 | μg | | 3 | μg | 2.44 | 0.60 | 3.70 | 0.65 | 3.41 | 0.71 | 2.16 | 0.84 |
| 7 | 10 | μg | | 16.5 | μg | 3.22 | 0.23 | 4.50 | 0.19 | 4.44 | 0.17 | 1.96 | 0.32 |
| 8 | 0.1 | μg | 1 μg | | | 3.44^{c} | 0.25 | 4.04^{d} | 0.69 | 2.73^{f} | 0.59 | 3.59^{c} | 0.79 |
| 9 | 1 | μg | 1 μg | | | 3.76^{c} | 0.14 | 4.47^{f} | 0.23 | 3.36 | 0.62 | 3.65^{d} | 0.40 |
| 10 | 10 | μg | 1 μg | | | 3.87^{c} | 0.35 | 4.29 | 0.71 | 3.47 | 0.59 | 3.48^{c} | 0.65 |
| 11 | 0.1 | μg | 1 μg | 1.65 | μg | 2.46^{c} | 1.54 | 3.58^{e} | 1.28 | 2.88^{f} | 1.16 | 2.71^{c} | 1.47 |
| 12 | 1 | μg | 1 μg | 3 | μg | 3.93^{c} | 0.45 | 4.69^{e} | 0.81 | 3.09 | 0.62 | 3.83^{d} | 0.77 |
| 13 | 10 | μg | 1 μg | 16.5 | μg | 4.26^{c} | 0.16 | 4.90^{e} | 0.11 | 3.79 | 0.37 | 4.15^{c} | 0.34 |

^{a}Peptide A is GVIDTSAVESAITDGQGDM (SEQ ID NO: 3)
^{b}A mean value of 0 indicates that no anti-peptide specific antibody was detected with a serum dilution of 1:20.
^{c}****
^{d}***
^{e}**
^{f}*

Anti-peptide A serum antibody titers in groups vaccinated with various doses of Conjugate A and Adjuvant A as well as alum are shown in Table 4a and 4b (14dp1, 14dp2 and 14dp3). Asterisks denote statistical significance versus the respective antigen alone control (group 2). A value of 0 indicates no peptide specific antibody was detected at a serum dilution of 1:20. Ordinary one-way ANOVA followed by Fisher's LSD were used to determine statistical analysis where*=p≤0.05, **=p≤0.01,***=p≤0.001 ****=p≤0.0001.

Groups that received Adjuvant A exhibited a significant boost in total IgG antibody response throughout all antigen doses at both 14dp1 and 14dp2 compared to groups that received the respective antigen dose alone (without adjuvant A). Groups receiving 1 μg of antigen and alum or 10 μg of antigen and alum compared to their respective antigen alone groups saw a modest antibody increase, however these increases were not statistically significant. Adding alum to Adjuvant A plus antigen did not lead to further increases in total IgG antibody titers (Groups 11-13). When comparing the group receiving 1 μg of antigen in combination with alum and Adjuvant A (Group 12), the antibody titers are statistically significantly higher than the group receiving 0.1 μg of antigen in combination with alum and Adjuvant A (Group 11). In addition, Group 12 receiving 1 μg of antigen in combination with alum and Adjuvant A had a slightly higher total antibody response compared to the 0.1 μg of antigen with Adjuvant A (Group 8), although this difference is not statistically significant. These data demonstrate that when both Adjuvant A and alum were used together as adjuvants, the high total antibody response was primarily driven by Adjuvant A with minimal increases (not significant) detected with the addition of alum.

After groups received a third injection, there were significantly higher total IgG antibody titer differences between groups receiving antigen in combination with Adjuvant A and groups receiving antigen alone. Collectively, these data demonstrate that two injections induce high total IgG antibody titers. Furthermore, adding alum to Adjuvant A-adjuvanted vaccines did not lead to a significant increase in total IgG antibody titers. Of note, high anti-peptide serum antibody titers (total IgG) were noted following a single vaccination (14dp1) with antigen+Adjuvant A suggesting a single-dose vaccine was effective in antigen naïve mice.

A third vaccination marginally boosted anti-CoaB peptide antibody titers. IgG2b overall were similar in magnitude and pattern as 14dp2 total IgG antibody titers. Anti-CoaB peptide IgG1 titers in groups having Adjuvant A with or without alum in combination with 0.1 μg of antigen exhibited higher antibody titers than the 0.1 μg antigen alone group. Anti-CoaB peptide IgG2b titers from groups receiving Adjuvant A with or without alum in combination with antigen (at all doses) also exhibited significantly higher antibody titers than the respective antigen alone group. These data suggest the high anti-CoaB peptide antibody response of these adjuvanted doses was independent of alum for both IgG1 and IgG2b. The anti-CoaB peptide IgG2b titers are highest at the 1 μg dose of Conjugate A in combination with Adjuvant A when compared to the other antigen doses in combination with Adjuvant A. The highest anti-CoaB peptide antibody response is from the group which was vaccinated with 10 μg of Conjugate A in combination with Adjuvant A and alum. Based on this collective data a high anti-CoaB peptide antibody titer can be achieved with 1 μg of Conjugate A in combination with Adjuvant A and a higher antibody titer can be achieved by increasing the antigen dose to 10 μg of Conjugate A and combining Adjuvant A and alum.

### Example 4

BALB/c and C57Bl6 mouse strain comparison study using Adjuvant A and Adjuvant B

In this study, Conjugate A with a copy number of 10.7 was used, and in some cases the conjugate was combined with a synthetic TLR4 adjuvant (Adjuvant A) in a 2.5% glycine formulation or with TLR7/8 adjuvant (Adjuvant B) in a glycerol formulation.

Female C57/BL/6 mice ages 6-8 weeks old or alternatively 6-8 week old Balb/C mice were vaccinated with Conjugate A and adjuvant dose as listed in the study design below on days 0 and 14. Peripheral blood was collected 14 days following the first vaccination by submandibular bleeds. On day 28 (14 days post-second) spleens and blood was collected via cardiac puncture on all mice.

TABLE 5
Anti-Peptide A ELISA^{a,b}

| | | Con- | | Ad- | | Ad- | | Total | | | |
|---|---|---|---|---|---|---|---|---|---|---|---|
| Group | | jugate | | juvant | | juvant | | IgG | | IgG1 | |
| # | Strain | A Dose | | A Dose | | B Dose | | Mean | SD | Mean | SD |
| 1 | Balb/C | 0 | | 0 | | 0 | | 0 | 0 | 0 | 0 |
| 2 | Balb/C | 1 | μg | 0 | | 0 | | 2.26 | 1.01 | 1.78 | 1.19 |
| 3 | Balb/C | 1 | μg | 0 | | 10 | μg | 3.90** | 0.97 | 2.92** | 1.52 |
| 4 | Balb/C | 1 | μg | 1 | μg | 0 | | 4.28*** | 0.75 | 4.03*** | 0.89 |
| 5 | Balb/C | 1 | μg | 1 | μg | 10 | μg | 4.09** | 2.03 | 3.66** | 0.72 |
| 6 | C57Bl6 | 0 | | 0 | | 0 | | 0 | 0 | 0 | 0 |
| 7 | C57Bl6 | 1 | μg | 0 | | 0 | | 2.37 | 1.33 | 2.28 | 1.74 |
| 8 | C57Bl6 | 1 | μg | 0 | | 10 | μg | 4.39*** | 0.37 | 1.41 | 1.29 |
| 9 | C57Bl6 | 1 | μg | 1 | μg | 0 | | 4.60**** | 0.80 | 2.88** | 1.62 |
| 10 | C57Bl6 | 1 | μg | 1 | μg | 10 | μg | 4.25*** | 0.67 | 3.14**** | 0.36 |

^{a}Peptide A is GVIDTSAVESAITDGQGDM (SEQ ID NO: 3)
^{b}A mean value of 0 indicates that no anti-peptide specific antibody was detected with a serum dilution of 1:20.

Table 5 shows Anti-Peptide A specific IgG antibody titers when adjuvanted with combinations of adjuvants in both mouse strains. Serum total IgG groups vaccinated with Conjugate A and adjuvanted with Adjuvant A and/or Adjuvant B. Asterisks denote statistical significance versus antigen alone in the respective mouse strain. A value of 0 indicates no peptide specific antibody was detected at a serum dilution of 1:20. Ordinary one-way ANOVA followed by Fisher's LSD were used to determine statistical analysis where*=p≤0.05, **=p≤0.01,***=p≤0.001 ****=p≤0.0001.

All mice vaccinated with Conjugate A and Adjuvant A or Adjuvant B had significantly higher anti-Peptide A antibody titers than groups receiving Conjugate A alone. Mice vaccinated with Conjugate A plus Adjuvant A (1 μg) trended towards higher titers than Adjuvant B (10 μg) but there was no significant difference between the titers of the compositions with the two adjuvants. Groups receiving Conjugate A in combination with both Adjuvant A and Adjuvant B did not respond with a higher antibody response than groups which had either adjuvant alone.

### Example 5

Multivalent Vaccine Using Peptides from Different Pf Phage Clades or Serotypes

Vaccines that contained multiple Pf phage coat protein peptides from various viral clades or serotypes lead to cross-reactive antibodies. Multivalent conjugation strategy and admixed multi-peptide strategy produce increased peptide specific antibody titers.

Three different phage Pf phage coat protein consensus peptides were conjugated to CRM. Peptide GVIDTSAVESAITDGQGDMC (SEQ ID NO: 36) was conjugated to carrier protein CRM-197 (Conjugate A). This conjugate had a copy number of 10.75. Peptide ESLLDETTKGVLAQASTDGC (SEQ ID NO: 37) was conjugated to CRM-197 (Conjugate B) and had a copy number of 10.13. Peptide GVIDTSAVEAAITEGKGDMC (SEQ ID NO: 38) was conjugated to CRM-197 (Conjugate C) and had a copy number of 10. For multivalent vaccines, SEQ ID NO: 36 and SEQ ID NO: 37 were conjugated to CRM-197 (Conjugate D) resulting in a total peptide copy number of 10.52. SEQ ID NO: 36, SEQ ID NO: 37, and SEQ ID NO: 38 were conjugated to CRM-197 (Conjugate E) resulting in a total peptide copy number of 10.99. Adjuvant A was added in a 2.5% glycine formulation.

### Anti-Peptide a Specific Antibody Titers

Female C57/BL6 mice, 6-8 weeks old, were vaccinated twice, IM, with 14 days between vaccinations. After the second vaccination, total IgG, IgG1 and IgG2b anti-Peptide A antibody titers from mice vaccinated with conjugates with or without adjuvant Adjuvant A were measured. Results are shown in Table 6. Asterisks denote statistical significance versus the respective antigen alone control. A value of 0 indicates no peptide specific antibody was detected at a serum dilution of 1:20. Ordinary one-way ANOVA followed by Fisher's LSD were used to determine statistical analysis where *=p≤0.05, **=p≤0.01, ***=p≤0.001, ****=p≤0.0001.

Mice receiving 1 μg Adjuvant A in combination with conjugates had significantly higher anti-Peptide A IgG antibodies than groups that received conjugates alone. Anti-Peptide A total IgG antibodies from serum from mice vaccinated with Conjugate C cross-reacted with Peptide A and exhibited high titers against Peptide A. As expected, no cross-reactive total IgG anti-Peptide A antibodies were generated from mice vaccinated with Conjugate B. Mice vaccinated with Conjugate D or Conjugates A+B (admixed) both had high total IgG anti-Peptide A antibodies but titers were lower than groups vaccinated with Conjugate A in combination with Adjuvant A or Conjugate C in combination with Adjuvant A. Groups vaccinated with Adjuvant A in combination with Conjugate E or admixed single-valent Conjugates A+B+C demonstrated high total IgG anti-Peptide A antibody responses similar in magnitude to those of mice vaccinated with monovalent Conjugate A, B, or C plus Adjuvant A. Total IgG Peptide A antibody responses were similar whether multivalent or admixed combination peptide-CRM antigens were used.

All groups receiving 1 μg Adjuvant A in combination with antigen had significantly higher IgG1 Peptide A antibodies than groups that received antigen alone. Mice vaccinated with Conjugate A exhibited the highest anti-Peptide A IgG1 antibodies when compared to all other groups. When comparing anti-Peptide A IgG1 antibody titers between groups vaccinated with Conjugate D, Conjugate E, Conjugate A+B (admixed), or Conjugate A+B+C (admixed), antibody titers were not significantly different.

All groups receiving 1 μg Adjuvant A in combination with antigen had significantly higher IgG2c anti-Peptide A antibodies than groups that received antigen alone. Similarly, groups vaccinated with Conjugate D, Conjugate E Conjugate A+B (admixed), or Conjugate A+B+C (admixed) demonstrate similar anti-Peptide B IgG2c antibody titers.

The conjugate doses used were based on a total CRM+conjugate concentration of 1 μg. (Each mouse was administered 1 ug of CRM (or CRM-conjugate) in 50 μl for each injection). As a result, the single-valent vaccine groups contained more peptide than the admixed or multivalent vaccine groups which potentially induced slightly lower titers observed in the multivalent and admixed groups.

TABLE 6
Peptide A ELISA^{d,e}

| Group | | Adjuvant | Total IgG | | IgG1 | | IgG2c | |
|---|---|---|---|---|---|---|---|---|
| # | Conjugateª | A^{c} | Mean | SD | Mean | SD | Mean | SD |
| 1 | N/A | − | 0 | 0 | 0 | 0 | 0 | 0 |
| 2 | A | − | 0 | 0 | 0 | 0 | 0 | 0 |
| 3 | B | − | 0 | 0 | 0 | 0 | 0.61 | 0.32 |
| 4 | C | − | 0 | 0 | 0 | 0 | 0 | 0 |
| 5 | E | − | 0 | 0 | 0 | 0 | 0.70 | 0.56 |
| 6 | A + B + C^{b} | − | 0 | 0 | 0 | 0 | 0 | 0 |
| 7 | D | − | 0 | 0 | 0 | 0 | 0 | 0 |
| 8 | A + B^{b} | − | 0 | 0 | 0 | 0 | 0 | 0 |
| 9 | A | + | 4.77**** | 0.52 | 3.71**** | 1.11 | 4.88**** | 0.66 |
| 10 | B | + | 0 | 0 | 0 | 0 | 0 | 0 |
| 11 | C | + | 5.35**** | 0.48 | 2.18**** | 1.23 | 3.65**** | 1.68 |
| 12 | E | + | 5.09**** | 0.41 | 3.18**** | 0.84 | 3.37**** | 1.36 |
| 13 | A + B + C^{b} | + | 4.77**** | 0.57 | 2.91**** | 1.24 | 3.22**** | 1.25 |
| 14 | D | + | 3.04**** | 1.36 | 2.31**** | 1.59 | 2.00** | 1.65 |
| 15 | A + B^{b} | + | 3.98**** | 0.48 | 2.93**** | 0.64 | 3.12**** | 1.67 |

^{a}All conjugates dose at 1 μg. In the case of admixed conjugates, the total combined dose of conjugate is 1 μg.
^{b}Conjugates admixed.
^{c}“−” = no Adjuvant A; “+” = 1 μg Adjuvant A.
^{d}Peptide A is GVIDTSAVESAITDGQGDM (SEQ ID NO: 3)
^{e}A mean value of 0 indicates that no anti-peptide specific antibody was detected with a serum dilution of 1:20.

### Anti-Peptide B Antibody Titers

Female C57/BL6 mice, 6-8 weeks old were vaccinated twice, IM, with 14 days between vaccinations. After the second vaccination, total IgG, IgG1 and IgG2b anti-Peptide B antibody titers from mice vaccinated with Conjugates A-E, with or without adjuvant Adjuvant A were measured. Results are shown in Table 7. Asterisks denote statistical significance versus the respective antigen alone control. A value of 0 indicates no peptide specific antibody was detected at a serum dilution of 1:20. Ordinary one-way ANOVA followed by Fisher's LSD were used to determine statistical analysis where *=p≤0.05, **=p≤0.01, ***=p≤0.001, ****=p≤0.0001.

Mice that were vaccinated with 1 μg Adjuvant A in combination with Conjugate B demonstrated significantly higher anti-Peptide B total IgG antibodies than groups which received Conjugate B alone. Mice vaccinated with either Conjugate A or Conjugate C in combination with Adjuvant A did not produce anti-Peptide B antibodies. Mice vaccinated with Conjugate D or Conjugates A+B (admixed), had high anti-Peptide B total IgG antibodies when Adjuvant A was present in the vaccine. Serum from mice vaccinated with the Conjugate D demonstrated slightly higher total IgG anti-Peptide B antibody titers than serum from mice vaccinated with admixed Conjugate A+B, when Adjuvant A is included in the vaccine. Mice vaccinated with Adjuvant A in combination with Conjugate A+B+C (admixed) had low anti-Peptide B total IgG response. The admixed strategy of Conjugates A+B+C in combination with Adjuvant A resulted in a higher anti-Peptide B total IgG response than Conjugate E.

Serum from mice vaccinated with Conjugate B in combination with 1 μg Adjuvant A demonstrated higher anti-Peptide B IgG1 than serum from mice vaccinated with Conjugate B alone. Serum from mice vaccinated with either Conjugate A or C in combination with Adjuvant A did not cross-react with Peptide B. Groups vaccinated with Adjuvant A in combination with Conjugate D or Conjugates A+B (admixed) had the highest anti-Peptide B IgG1 antibodies compared to all other groups. Anti-Peptide B IgG1 titers from the multivalent group were slightly elevated compared to anti-Peptide B IgG1 titers from the admixed group. Serum from mice vaccinated with Adjuvant A in combination with Conjugate E or Conjugates A+B+C (admixed) had lower anti-Peptide B IgG1 antibodies compared to other groups vaccinated with conjugates in combination with Adjuvant A, likely due to ⅓ dose of peptide used in the combination groups. Mice receiving 1 μg Adjuvant A in combination with Conjugate B had higher anti-Peptide B IgG2c antibodies than the group with no adjuvant. Conjugate B was most immunogenic as a bi-valent vaccine in combination with Peptide A (Conjugate D), resulting in a higher anti-Peptide B antibody response than the monovalent conjugate containing only Peptide B. However, when used as a triple-valent vaccine (Conjugate E) responses were greatly reduced. This suggests some optimization may be necessary to obtain the correct peptide copy number and combination using both multivalent and admixed approaches to complete coverage and immunity to Pseudomonas aeruginosa.

TABLE 7
Anti-Peptide B ELISA^{d,e}

| Group | | Adjuvant | Total IgG | | IgG1 | | IgG2c | |
|---|---|---|---|---|---|---|---|---|
| # | Conjugateª | A^{c} | Mean | SD | Mean | SD | Mean | SD |
| 1 | N/A | − | 0 | 0 | 0 | 0 | 0 | 0 |
| 2 | A | − | 0 | 0 | 0 | 0 | 0 | 0 |
| 3 | B | − | 0 | 0 | 0 | 0 | 0 | 0 |
| 4 | C | − | 0 | 0 | 0 | 0 | 0 | 0 |
| 5 | E | − | 0 | 0 | 0 | 0 | 0 | 0 |
| 6 | A + B + C^{b} | − | 0 | 0 | 0 | 0 | 0 | 0 |
| 7 | D | − | 0 | 0 | 0 | 0 | 0 | 0 |
| 8 | A + B^{b} | − | 0 | 0 | 0 | 0 | 0 | 0 |
| 9 | A | + | 0 | 0 | 0 | 0 | 0 | 0 |
| 10 | B | + | 2.02**** | 2.16 | 0.81**** | 0.88 | 1.43** | 1.85 |
| 11 | C | + | 0 | 0 | 0 | 0 | 0 | 0 |
| 12 | E | + | 0 | 0 | 0 | 0 | 0 | 0 |
| 13 | A + B + C^{b} | + | 1.56* | 1.48 | 0 | 0 | 0 | 0 |
| 14 | D | + | 3.72**** | 1.56 | 1.31**** | 1.50 | 2.60**** | 1.82 |
| 15 | A + B^{b} | + | 1.51* | 1.46*** | 0 | 0 | 1.19* | 1.28 |

^{a}All conjugates dose at 1 μg. In the case of admixed conjugates, the total combined dose of conjugate is 1 μg.
^{b}Conjugates admixed.
^{c}“−” = no Adjuvant A; “+” = 1 μg Adjuvant A.
^{d}Peptide B is ESLLDETTKGVLAQASTDG (SEQ ID NO: 22)
^{e}A mean value of 0 indicates that no anti-peptide specific antibody was detected with a serum dilution of 1:20.

### Anti-Peptide C Antibody Titers

Female C57/BL6 mice, 6-8 weeks old were vaccinated twice, IM, with 14 days between vaccinations. After the second vaccination, total IgG, IgG1 and IgG2b anti-Peptide C antibody titers from mice vaccinated with Conjugates A-E, with or without Adjuvant A, were measured. Results are shown in Table 8. Asterisks denote statistical significance versus the respective antigen alone control. A value of 0 indicates no peptide specific antibody was detected at a serum dilution of 1:20. Ordinary one-way ANOVA followed by Fisher's LSD were used to determine statistical analysis where *=p≤0.05, **=p≤0.01, ***=p≤0.001, ****=p≤0.0001.

Mice vaccinated with 1 μg Adjuvant A in combination with Conjugate C demonstrated significantly higher anti-Peptide C total IgG antibodies than groups which received antigen alone. Serum anti-Peptide C total IgG antibodies from mice vaccinated with Conjugate A with Adjuvant A exhibited high titers, but no total IgG anti-Peptide C antibodies were generated from mice vaccinated with Conjugate B, with or without Adjuvant A. Mice vaccinated with Conjugate D or E, with Adjuvant A, both had high total IgG anti-Peptide C antibody titers. However, multivalent or admixed conjugates resulted in anti-Peptide C titers that were lower than groups vaccinated with monovalent Conjugate A or C in combination with Adjuvant A. Groups vaccinated with Adjuvant A in combination with Conjugate E or admixed Conjugates A+B+C, had high total IgG anti-Peptide C antibody responses and to a similar magnitude as mice vaccinated with monovalent Conjugate C. When comparing groups vaccinated with conjugates in the multivalent form versus admixed form, total IgG anti-Peptide C antibody responses were similar.

Mice vaccinated with 1 μg Adjuvant A in combination with antigen had significantly higher anti-Peptide C IgG1 antibodies than groups receiving antigen alone except for mice vaccinated with Conjugate B which generated no measurable anti-Peptide C antibody titers. Mice vaccinated with Adjuvant A in combination with monovalent Conjugate A or Conjugate C had high anti-Peptide C IgG1 antibodies. Mice vaccinated with Adjuvant A in combination with Conjugate E or admixed Conjugates A+B+C had serum IgG1 anti-Peptide C titers of similar magnitude when compared to serum from mice vaccinated with the monovalent Conjugates A and C. Serum from mice vaccinated with the bivalent conjugates (admixed or multivalent) had overall lower anti-Peptide C IgG1 and IgG2c antibody levels, likely due to the lower overall peptide dose.

TABLE 8
Anti-Peptide C ELISA^{d,e}

| Group | | Adjuvant | Total IgG | | IgG1 | | IgG2c | |
|---|---|---|---|---|---|---|---|---|
| # | Conjugateª | A^{c} | Mean | SD | Mean | SD | Mean | SD |
| 1 | N/A | − | 0 | 0 | 0 | 0 | 0 | 0 |
| 2 | A | − | 0 | 0 | 0 | 0 | 0 | 0 |
| 3 | B | − | 0 | 0 | 0 | 0 | 0 | 0 |
| 4 | C | − | 0 | 0 | 0 | 0 | 0 | 0 |
| 5 | E | − | 0 | 0 | 0 | 0 | 0 | 0 |
| 6 | A + B + C^{b} | − | 0 | 0 | 0 | 0 | 0 | 0 |
| 7 | D | − | 0 | 0 | 0 | 0 | 0 | 0 |
| 8 | A + B^{b} | − | 0 | 0 | 0 | 0 | 0 | 0 |
| 9 | A | + | 4.875 | 0.5439 | 3.254 | 1.258 | 4.482 | 0.6661 |
| 10 | B | + | 0 | 0 | 0 | 0 | 0 | 0 |
| 11 | C | + | 5.444 | 0.4317 | 3.631 | 1.348 | 4.726 | 0.6432 |
| 12 | E | + | 4.939 | 0.3327 | 3.64 | 0.6134 | 4.296 | 0.6182 |
| 13 | A + B + C^{b} | + | 4.847 | 0.5252 | 3.133 | 1.241 | 4.069 | 0.7667 |
| 14 | D | + | 2.999 | 1.623 | 0.869 | 1.044 | 1.959 | 1.595 |
| 15 | A + B^{b} | + | 4.035 | 0.5256 | 1.938 | 1.578 | 2.162 | 1.833 |

^{a}All conjugates dose at 1 μg. In the case of admixed conjugates, the total combined dose of conjugate is 1 μg.
^{b}Conjugates admixed.
^{c}“−” = no Adjuvant A; “+” = 1 μg Adjuvant A.
^{d}Peptide C is GVIDTSAVEAAITEGKGDM (SEQ ID NO: 4)
^{e}A mean value of 0 indicates that no anti-peptide specific antibody was detected with a serum dilution of 1:20.

Overall these data clearly demonstrate that mice vaccinated with a multivalent peptide vaccine (admixed individual conjugates or co-conjugated to the same CRM) targeting different Pf phage clades generates a robust immune response to multiple clades at the same time. Adjuvant A in combination with multivalent conjugates significantly boosted the immune response to all peptide-CRM conjugates. Peptide A and Peptide C share 84.21% sequence identity, while Peptide A and Peptide B or Peptide B and Peptide C share only 5.26% sequence identity. A multivalent vaccine targeting the major clades of Pf phage in Pseudomonas aeruginosa disclosed herein is a viable vaccine candidate for the treatment or prevention of Pseudomonas aeruginosa infection.

### Example 6

A wealth of pre-clinical literature exists on the efficacy of vaccines against bacterial pathogens (see Mendoza N, Ravanfar P, Satyaprakash A, Pillai S, Creed R. Existing antibacterial vaccines. Dermatol Ther 2009; 22:129-42 for a review). However, there is no approved vaccine that is effective against Pa infections due to mutations or diversity of the target antigen. Microbes mutate rapidly, which can result in vaccine escape mutations (Weissman D, Alameh M G, de Silva T, et al. D614G Spike Mutation Increases SARS CoV-2. Susceptibility to Neutralization. Cell Host Microbe 2021; 29:23-31 e4.; Krammer F. The human antibody response to influenza A virus infection and vaccination. Nat Rev Immunol 2019; 19:383-97.) For example, mutations in bacterial surface antigens such as the OspA surface protein of Borrelia burgdorferi (Lyme disease) can lead to vaccine escape (Sole M, Bantar C, Indest K, et al. Borrelia burfgdorferi escape mutants that survive in the presence of antiserum to the OspA vaccine are killed when complement is also present. Infect Immun 1998; 66:2540-6.) Viruses such as influenza mutate at an even faster rate than bacteria, readily acquiring escape mutations that necessitate seasonal vaccinations (Petrova V N, Russell C A. The evolution of seasonal influenza viruses. Nat Rev Microbiol 2018; 16:47-60.) The Pf prophage mutates at a rate comparable to ssRNA viruses, ˜1,000× faster than the rest of the bacterial chromosome (McElroy K E, Hui J G, Woo J K, et al. Strain-specific parallel evolution drives short-term diversification during Pseudomonas aeruginosa biofilm formation. P Natl Acad Sci USA 2014; 111:E1419-27.).

Interestingly, the Pf phage gene encoding the target CoaB antigen has low sequence variability in nature. There are at least 4,955 Pa isolates in the pseudomonas.com database (Winsor G L, Griffiths E J, Lo R, Dhillon B K, Shay J A, Brinkman F S. Enhanced annotations and features for comparing thousands of Pseudomonas genomes in the Pseudomonas genome database Nucleic Acids Res 2016; 44:D646-53). When the entire Pf genome is examined, Pf phage can be divided into two major lineages (Fiedoruk K, Zakrzewska M, Daniluk T, Piktel E, Chmielewska S, Bucki R. Two Lineages of Pseudomonas aeruginosa Filamentous Phages: Structural Uniformity over Integration Preferences. Genome Biol Evol 2020; 12:1765-81.). Amongst these Pf genomes, the CoaB gene is well conserved.

Phage variants were organized into clades using commercially available software as described above, specifically, ClustalW with default parameters (Pairwise Alignment: Alignment Speed=Slow, Open Gap Penalty=15, and Extend Gap Penalty=6.66; Multiple Alignment: Open Gap Penalty=15, and Extend Gap Penalty=6.66; Delay Divergent=30%, and Transitions=Weighted), as described by Thompson et al. in Current Protocols in Bioinformatics 2003, vol 00 (1) pp. 2.3.1-2.3.22 (doi.org/10.1002/0471250953.bi0203s00). software.

Out of 4,955 Pa isolates in the pseudomonas.corn database, CoaB isoforms group into only two major clades, CoaBa and CoaBb. And within each clade, sequence variability is also low; as each clade contains only two subclades CoaBa1/CoaBa2 and CoaBb1/CoaBb2. Clade consensus sequences and subclade consensus sequences are shown in Table YY. The consensus CoaBa hapten sequence (GSVIDTSAVESAITDGQGD) was present at 2,400 occurrences while consensus CoaBb hapten sequence (AESLLDETTKEVLTQAGTDG) was present at 1,779 occurrences. Collectively, out of the 4,955 total Pa strains, the two lead hapten sequences are present in 4,179 Pa strains (84%). These observations indicate that despite the high mutation rate of the Pf phage genome, surprisingly, the gene encoding the CoaB epitope is itself evolutionarily constrained and remarkably stable.

TABLE 9

| | | | | | | | | | | | | | | | | | | | | |
|---|---|---|---|---|---|---|---|---|---|---|---|---|---|---|---|---|---|---|---|---|
| CLADE A | | | | | | | | | | | | | | | | | | | | |
| CoaB a1 | | G | V | I | D | T | S | A | V | E | S | A | I | T | D | G | Q | G | D | |
| CoaB a2 | G | S | V | I | D | T | S | A | V | E | A | A | I | T | E | G | K | G | D | |
| Clade A | G | S* | V | I | D+ | T* | S* | A | V | E+ | S* | A | I | T* | D+ | G | Q′ | G | D+ | M# |
| Consensus | | | | | | | | | | | | | | | | | | | | |
| CoaB-a | | | | | | | | | | | | | | | | | | | | |
| CLADE B | | | | | | | | | | | | | | | | | | | | |
| CoaB b1 | A | D | S | L | I | D | E | T | T | K | E | V | L | T | Q | A | G | T | D | G |
| CoaB b2 | A | E | S | L | I | D | E | T | T | K | G | V | L | A | Q | A | S | T | D | G |
| Clade B | A | E+ | S* | L | L | D+ | E+ | T* | T* | K″ | E | V | L | T | Q′ | A | G | T* | D+ | G |
| Consensus | | | | | | | | | | | | | | | | | | | | |
| CoaB-b | | | | | | | | | | | | | | | | | | | | |

(*) = hydroxylic;
(+) = acidic;
(′) = amidic;
(″) = basic;
(#) = sulfur containing

Table 10 presents CoaB epitope amino acid sequence alignment of target antigens CoaBa1, CoaBa2, CoaBb1, and CoaBb2. The CoaB epitope offers an ideal multivalent vaccine target.

TABLE 10

| | | | | | | | | | | | | | | | | | | | | | |
|---|---|---|---|---|---|---|---|---|---|---|---|---|---|---|---|---|---|---|---|---|---|
| CoaB | G | − | V | I | D+ | T* | S* | A | V | E+ | S* | A | I | T* | D+ | G | Q′ | G | D+ | − | Isoform |
| a1 | | | | | | | | | | | | | | | | | | | | | A |
| CoaB | G | S* | V | I | D+ | T* | S* | A | V | E+ | A | A | I | T* | E+ | G | K″ | G | D+ | − | |
| a2 | | | | | | | | | | | | | | | | | | | | | |
| CoaB | A | D+ | S* | L | I | D+ | E+ | T* | T* | K″ | E+ | V | L | T* | Q′ | A | G | T* | D+ | G | Isoform |
| b1 | | | | | | | | | | | | | | | | | | | | | |
| CoaB | A | E+ | S* | L | I | D+ | E+ | T* | T* | K″ | G | V | L | A | Q′ | A | S* | T* | D+ | G | B |
| b2 | | | | | | | | | | | | | | | | | | | | | |

### Example 7

### Modified Phage CoaB Peptide Sequences and Conjugates

Phage CoaB peptide sequences shown in Table 11 were commercially synthesized. Select amino acid sequences were modified to have a cysteine residue at the C-terminal of the sequence. Certain of the peptides with the cysteine modification at the C-terminal were subsequently coupled to CRM197 using a GMBS linker strategy according to the process described above (in Example 1). Table 11 shows which peptide sequences were conjugated to CRM197. For example, SEQ ID 3, SEQ. ID. 4 and SEQ. ID. 22, were each modified with a cysteine to provide SEQ. ID. 36, SEQ. ID. 38 and SEQ. ID. 37 respectively and coupled to CRM197. Additionally, certain peptide sequences were modified with a cysteine residue at the N-terminal, the C-terminal or in the middle of the sequence (see. Ref. Nos. c5.1, 5.1c, and 5.cd respectively) which may provide varied presentations of the epitope peptide when conjugated to a carrier.

TABLE 11

| Reference | Sequence and | |
|---|---|---|
| No. | (SEQ. ID.) | Conjugate |
| 1.1 | GVIDTSAVESAITDGQGDM | |
| | (SEQ ID: 3) | |
| 1.1c | GVIDTSAVESAITDGQGDMC | yes |
| | (SEQ ID: 36) | |
| 1.2 | GVIDTSAVEAAITEGKGDM | |
| | (SEQ ID: 4) | |
| 1.2c | GVIDTSAVEAAITEGKGDMC | yes |
| | (SEQ ID: 38) | |
| 0.1 | DSLIDETTKEVLTQAGTDG | |
| | (SEQ ID: 48) | |
| 0.1c | DSLIDETTKEVLTQAGTDGC | yes |
| | (SEQ ID: 62) | |
| 2.1 | AESLLDETTKGVLAQASTDG | |
| | (SEQ ID: 49) | |
| 2.1c | AESLLDETTKGVLAQASTDGC | yes |
| | (SEQ ID: 63) | |
| 3.1 | DYSSLTSDVDFSTIATG | |
| | (SEQ ID: 50) | |
| 3.1c | GWDYSSLTSDVDFSTIATGC | yes |
| | (SEQ ID: 64) | |
| 4.1 | AEGDIDTTKALAYIAGGLT | |
| | (SEQ ID: 52) | |
| 4.1c | AEGDIDTTKALAYIAGGLTC | yes |
| | (SEQ ID: 65) | |
| 5.1 | ESLLDETTKGVLAQASTDG | |
| | (SEQ ID: 22) | |
| 5.1c | ESLLDETTKGVLAQASTDGC | yes |
| | (SEQ ID: 37) | |
| c5.1 | CESLLDETTKGVLAQASTDG | |
| | (SEQ ID: 53) | |
| 5.cl | ESLLDETTKCVLAQASTDG | |
| | (SEQ ID: 54) | |
| 0.1k Biotin | DSLIDETTKEVLTQAGTDGK | |
| | (SEQ ID: 55) + Biotin | |
| 1.1k Biotin | GVIDTSAVESAITDGQGDMK | |
| | (SEQ ID: 56) + Biotin | |

### Example 8

Anti-CoaB Phage Polyclonal Antibodies Inhibit P. aeruginosa Growth In Vitro

The indicated bacterial strains were incubated in 50 μL serum collected from naïve or vaccinated mice. Growth was measured every 15 minutes by optical density at 600 nm (OD600). Pa strain PAO1 is infected by a Pf phage strain Pf4 whereas Pa ΔPf4 was genetically cured of its Pf4 infection. Results are the mean of quadruplicate measurements shown in Table 12.

TABLE 12

| Sample | Mean P. aeruginosa growth (OD600) | SD |
|---|---|---|
| PAO1 + CoaBa pAb | 3.11 | 0.11 |
| ΔPf4 + CoaBa pAb | 5.95 | 1.66 |
| PAO1 + naïve serum | 4.2 | 1.29 |
| ΔPf4 + naïve serum | 5.98 | 1.39 |

### Example 9

### Adjuvant System for the CoaB-CRM Conjugate

The lead CoaB vaccines consist of a peptide hapten conjugated to CRM197. To determine whether TLR7 and TLR4 adjuvants would provide a beneficial effect on antibody titres or provide a dose sparing effect, female C57/BL/6 mice aged 6-8 weeks old (10 per group) were vaccinated intramuscularly with 1 μg of CoaBa-CRM conjugate in combination with indicated dose of (Adjuvant B), a TLR 7/8 adjuvant, and or (Adjuvant A) a TLR4 adjuvant. A booster with respective dose of adjuvant combination with 1 g of CoaBa-CRM conjugate was administered 14 days post primary. ELISA data is from serum collected 14 days post-secondary vaccination boost. Interestingly, 1 μg Adjuvant A alone produced the best dose sparing results. The vaccine dose experiments demonstrated that the TLR4 agonist Adjuvant A produced the highest antibody titers against the CoaBa1 hapten and was dose-sparing (Table 13).

TABLE 13

| Sample | Mean Log10 total serum IgG titer | SD |
|---|---|---|
| Naïve | 0.01 | 0.01 |
| 1 μg conjugate | 2.83 | 1.77 |
| 0.1 μg Adj B | 2.78 | 0.87 |
| 0.01 μg Adj A | 3.74 | 1.02 |
| 0.01 μg Adj A + 0.1 μg Adj B | 4.82 | 1.43 |
| 1 μg Adj B | 3.21 | 1.8 |
| 0.1 μg Adj A | 4.55 | 0.95 |
| 0.1 μg Adj A + 1 μg Adj B | 4.65 | 0.76 |
| 10 μg Adj B | 2.97 | 0.74 |
| 1 μg Adj A | 5.12 | 0.68 |
| 1 μg Adj A + 10 μg Adj B | 4.97 | 0.87 |
| Positive Control | 4.57 | 0.24 |
| Negative Control | 0.46 | 0.15 |

### Example 10

Anti-CoaBa IgG Subtypes Induced with TLR4 Agonist

Female C57/BL/6 mice aged 6-8 weeks old (5 per group) were vaccinated intramuscularly with 1 τg of CoaBa-CRM conjugate in combination with indicated dose of TLR4 agonist Adjuvant A. A booster with respective dose of adjuvant combination with 1 μg of CoaBa-CRM conjugate was administered 14 days post primary. ELISA data are from serum collected 14 days postsecondary vaccination boost. (A) Total IgG, (B) IgG1, (C) IgG2a isotypes are shown. In dose experiments, IgG subtype analysis showed that adjuvanting CoaBa with Adjuvant A elicited both IgG1 and IgG2b subclasses, suggesting that this vaccine formulation could provide protection against PA via both neutralization and antibody-mediated effector functions involving Fc gamma receptors (Table 14).

TABLE 14

| | Mean | | Mean | | Mean | |
|---|---|---|---|---|---|---|
| | total IgG | SD | IgG1 | SD | IgG2b | SD |
| Naive | 0.50 | 0.00 | 0.50 | 0.00 | 0.50 | 0.00 |
| 0.0001 μg Adj A | 1.27 | 1.23 | 0.95 | 0.64 | 1.02 | 1.16 |
| 0.001 μg Adj A | 2.38 | 0.97 | 1.86 | 1.30 | 1.22 | 1.20 |
| 0.01 μg Adj A | 3.15 | 0.57 | 1.99 | 0.59 | 2.45 | 1.29 |
| 0.1 μg Adj A | 4.00 | 0.78 | 2.45 | 1.85 | 3.90 | 0.90 |
| 1 μg Adj A | 4.19 | 0.57 | 3.60 | 0.57 | 4.31 | 0.46 |
| 10 μg Adj A | 4.25 | 0.73 | 2.88 | 1.43 | 3.31 | 1.62 |
| Positive | 4.38 | 0.06 | 3.67 | 0.21 | 3.99 | 0.03 |
| negative | 0.50 | 0.00 | 0.50 | 0.00 | 0.50 | 0.00 |

### Example 11

CoaBa1 and CoaBa2 Conjugate Vaccines Produce within-Clade Cross Reactive Immunity

Female C57/BL/6 mice aged 6-8 weeks old (8 per group) were vaccinated intramuscularly with 1 μg of CoaBa-CRM or 1 g of CoaBb-CRM conjugate in combination with 1 g dose of TLR 4 agonist Adjuvant A. A booster with 1 μg dose of Adjuvant A adjuvant combination with 1 μg of respective conjugate was administered 14 days post primary. ELISA data is from serum collected 14 days post-secondary vaccination boost. CoaBa1 within-clade cross reactive immunity against coaBa2 was determined. (Tables 15 and 16). Clade A CoaBa cross-reactive immunity against Clade B CoaBb epitope was determined. (Table 17).

Immunity against CoaB subtypes produces within-clade crossreactive immunity; vaccinating with CoaBa1-CRM197 produced cross-reactivity to CoaBa2-CRM197 (Table 15), and CoaBa2-CRM197 produced cross-reactivity to CoaBa1-CRM197 (Table 16). Similarly, a multivalent CoaBa1-CRM197-CoaBa2 vaccine produced immunity against both clade A subtypes (Tables 15 and 16). However, vaccinating with CoaBb1 did not produce cross-reactive immunity to clade A subtypes CoaBa1. (Table 17). These results indicate that a multivalent vaccine composed of haptens targeting the CoaBa and CoaBb consensus sequences will broadly recognize the CoaB epitopes found in the diverse Pa isolates that cause human infection.

TABLE 15

| | Total IgG | |
|---|---|---|
| | ELISA plate | |
| | coated with | |
| Sample | CoaBa1 | SD |
| Naïve | 0.50 | 0.00 |
| CoaBa1 | 0.50 | 0.00 |
| CoaBa1 + Adj A | 4.87 | 0.54 |
| CoaBa2 | 0.50 | 0.00 |
| CoaBa2 + Adj A | 5.44 | 0.43 |
| CoaBa1/CoaBa2 admixed | 0.50 | 0.00 |
| CoaBa1/CoaBa2 admixed + Adj A | 3.90 | 0.39 |
| CoaBa1/CoaBa2 multivalent | 0.50 | 0.00 |
| CoaBa1/CoaBa2 multivalent + Adj A | 1.970203625 | 0.9 |

TABLE 16

| | Total IgG | |
|---|---|---|
| | ELISA plate coated | |
| Sample | with CoaBa2 | SD |
| Naïve | 0.5 | 0 |
| CoaBa2 | 0.5 | 0 |
| CoaBa2 + Adj A | 4.93 | 0.51 |
| CoaBa1 | 0.5 | 0 |
| CoaBa1 + Adj A | 5.3 | 0.4 |
| CoaBa1/CoaBa2 admixed | 0.50 | 0.00 |
| CoaBa1/CoaBa2 admixed + Adj A | 4.03 | 0.53 |
| CoaBa1/CoaBa2 multivalent | 0.50 | 0.00 |
| CoaBa1/CoaBa2 multivalent + Adj A | 3.00 | 1.62 |

TABLE 17

| | Total | |
|---|---|---|
| | IgG | |
| | ELISA | |
| | plate | |
| | coated | |
| | with | |
| Sample | CoaBb1 | SD |
| Naïve | 0.5 | 0 |
| CoaBa1 | 0.5 | 0 |
| CoaBa1 + Adj A | 0.5 | 0 |
| CoaBb1 | 0.5 | 0 |
| CoaBb1 + Adj A | 2.36 | 1.66 |

The foregoing discussion discloses and describes merely exemplary embodiments of the invention. One skilled in the art will readily recognize from such discussion and from the accompanying drawings and claims, that various changes, modifications and variations can be made therein without departing from the spirit and scope of the invention as defined in the following claims.

1. An immunogenic composition comprising:
a first Pf bacteriophage peptide, or derivative thereof;
a second Pf bacteriophage peptide, or derivative thereof; wherein the second Pf bacteriophage peptide, or derivative thereof, is different from the first bacteriophage peptide or derivative thereof; and
one or more carriers;
wherein the Pf bacteriophage peptides, or derivatives thereof, are independently coupled to the one or more carriers.

2. The immunogenic composition of claim 1 comprising:
(i) a first conjugate comprising the first Pf bacteriophage peptide, or derivative, coupled to a first carrier; and
a second conjugate comprising the second Pf bacteriophage peptide, or derivative, coupled to a second carrier; or
(ii) an immunogenic conjugate comprising the first and second Pf bacteriophage peptides, or derivatives thereof, coupled to a first carrier.

3. (canceled)

4. The immunogenic composition of claim 1, wherein:
(i) each Pf bacteriophage peptide is a Pf4 bacteriophage peptide;
(ii) each Pf bacteriophage peptide comprises an acidic region of a CoaB coat protein of a Pf bacteriophage;
(iii) each Pf bacteriophage peptide, or derivative thereof, independently comprises from 20-25% acidic residues; or
(vi) each Pf bacteriophage peptide, or derivative thereof, is between 12-25 amino acids in length.

5-7. (canceled)

8. The immunogenic composition of claim 1, wherein the first Pf bacteriophage peptide, or derivative, comprises or consists of a peptide of formula (A)
A1-A2-A3-A4-A5-A6-A7-A8-A9-A10-All-A12-A13-A14-A15-A16-A17-A18-(A)
wherein
A9, A14, and A18 are each independently an acidic amino acid residue;
A4 is an acidic or neutral amino acid residue;
A1-A3, A5-A8, A10-A13, A15, and A17 are each independently a neutral amino acid residue; and
A16 is a neutral or basic amino acid residue;
wherein the first Pf bacteriophage peptide, or derivative, is attached at A18,
wherein:
(i) A1-A3, A5-A8, A10-A13, A15, and A17 are each independently a glycine, alanine, serine, valine, isoleucine, threonine, or glutamine residue; and
A16 is a glutamine or lysine residue; or
(ii) A1 is a glycine, alanine, or serine residue;
A2 is a valine residue;
A3 is an isoleucine residue;
A5 is a threonine residue;
A6 is a serine residue;
A7 is an alanine or serine residue;
A8 is a valine residue;
A10 is a serine, alanine, or a glutamine residue;
All is an alanine residue;
A12 is an isoleucine residue;
A13 is a threonine residue;
A15 is a glycine residue;
A16 is a glutamine or lysine residue; and
A17 is a glycine or serine residue.

9-10. (canceled)

11. The immunogenic composition of claim 8, wherein:
(i) A4 is an acidic amino acid residue;
(ii) A9, A14, and A18 are each independently an aspartic acid or glutamic acid residue;
(iii) A9 is a glutamic acid residue;
(iv) A14 is an aspartic acid residue or a glutamic acid residue;
(v) A18 is an aspartic acid residue; or
(vi) A4 is an aspartic acid residue or a glycine residue.

12-19. (canceled)

20. The immunogenic composition of claim 1, wherein the first Pf bacteriophage peptide, or derivative, comprises or consists of a peptide DTSAVEX¹AITX²GX³GD (SEQ ID NO: 34) wherein:
X¹ is a neutral amino acid residue;
X² is an acidic amino acid residue; and
X³ is a neutral or basic amino acid residue;
or
X¹ is a serine, alanine, or glutamine residue;
X² is an aspartic acid or glutamic acid residue; and
X³ is a glutamine or lysine residue.

21. (canceled)

22. The immunogenic composition of claim 20, wherein:
(i) the first Pf bacteriophage peptide, or derivative, comprises or consists of a peptide X^{a}X^{b}DTSAVEX¹AITX²GX³GD (SEQ ID NO: 57) wherein:
X^{a} and X^{b} are each independently a neutral amino acid residue;
(ii) the first Pf bacteriophage peptide, or derivative, comprises or consists of a peptide X^{a}X^{b}DTSAVEX¹AITX²GX³GDM (SEQ ID NO: 58) wherein:
X^{a} and X^{b} are each independently a neutral amino acid residue; or
(iii) the first Pf bacteriophage peptide, or derivative, comprises or consists of a peptide YX^{a}X^{b}DTSAVEX¹AITX²GX³GD (SEQ ID NO: 59) wherein:
Y, X^{a} and X^{b} are each independently a neutral amino acid residue.

23-24. (canceled)

25. The immunogenic composition of claim 24, wherein Y is a glycine, alanine, or serine residue or a glycine-serine diamino acid residue.

26. (canceled)

27. The immunogenic composition of claim 22, wherein:
X^{a} and X^{b} are each independently a valine or isoleucine residue; or
X^{a} is a valine residue and X^{b} is an isoleucine residue.

28. (canceled)

29. The immunogenic composition of claim 1, wherein the first Pf bacteriophage peptide, or derivative, comprises or consists of a peptide selected from the group consisting of:

| (SEQ ID NO: 3) | |
|---|---|
| | GVIDTSAVESAITDGQGDM; |
| (SEQ ID NO: 4) | |
| | GVIDTSAVEAAITEGKGDM; |
| (SEQ ID NO: 6) | |
| | GVIDTSAVEAAITDGQGDM; |
| (SEQ ID NO: 7) | |
| | AVIDTSAVESAITDGQGDM; |
| (SEQ ID NO: 8) | |
| | SVIDTSSVEQAITDGKSDM; |
| (SEQ ID NO: 39) | |
| | GSVIDTSAVESAITDGQGDM; |
| (SEQ ID NO: 40) | |
| | GVIDTSAVESAITEGQGDM; |
| (SEQ ID NO: 41 | |
| | GSVIDTSAVEAAITEGKGDM. |
| (SEQ ID NO: 42) | |
| | GSVIDTSAVESAITDGQGD; |
| (SEQ ID NO: 43) | |
| | GVIDTSAVESAITDGOGD; |
| | and |
| (SEQ ID NO: 44) | |
| | GSVIDTSAVEAAITEGKGD. |

30. The immunogenic composition of claim 1, wherein the second Pf bacteriophage peptide, or derivative, comprises or consists of a peptide of formula (B)
B2-B3-B4-B5-B6-B7-B8-B9-B10-B11-B12-B13-B14-B15-B16-B17-B18-B19-B20-B21- (B)
wherein
B3, B7, B8, and B20 are independently an acidic amino acid residue;
B12 is an acidic or neutral amino acid residue;
B2, B4-B6, B9, B10, B13-B19, and B21 are each independently a neutral amino acid residue; and
B11 is a basic amino acid residue;
wherein the second Pf bacteriophage peptide, or derivative, is attached at B21; or
wherein:
(i) B2, B4-B6, B9, B10, B13-B19, and B21 are each independently a phenylalanine, alanine, serine, leucine, isoleucine, threonine, valine, glutamine, or glycine residue; and
B11 is a lysine residue; or
(ii) B2 is an alanine residue:
B4 is a serine residue;
B5 is a leucine residue:
B6 is a leucine or isoleucine residue;
B9 is threonine residue:
B10 is a threonine residue;
B11 is a lysine residue:
B13 is a valine or alanine residue;
B14 is a leucine residue:
B15 is a threonine or alanine residue;
B16 is a glutamine residue;
B17 is an alanine residue;
B18 is a glycine or serine residue;
B19 is a threonine residue; and
B21 is a glycine residue.

31-32. (canceled)

33. The immunogenic composition of claim 30, wherein:
(i) B12 is an acidic amino acid residue or a neutral amino acid residue;
(ii) B3, B7, B8, and B20 are each independently an aspartic acid or glutamic acid residue;
(iii) B3 is an aspartic acid residue or a glutamic acid residue;
(iv) B7 is an aspartic acid residue;
(v) B8 is a glutamic acid residue;
(vi) B20 is an aspartic acid residue; or
(vii) B12 is a glutamic acid residue or a glycine residue.

34-42. (canceled)

43. The immunogenic composition of claim 1, wherein the second Pf bacteriophage peptide, or derivative, comprises or consists of a peptide X⁴SLX⁵DETTKX⁶VLX⁷QAX⁸TD (SEQ ID NO: 35) wherein
X⁵, X⁷, and X⁸ are independently a neutral amino acid residue;
X⁴ is an acidic amino acid residue; and
X⁶ is a neutral or acidic amino acid residue; or
wherein:
(i) X⁴ is an aspartic acid or glutamic acid residue;
X⁵ is a leucine or isoleucine residue;
X⁶ is glycine or glutamic acid residue;
X⁷ is threonine or alanine residue; and
X⁸ is glycine or serine residue; or
(ii) the second Pf bacteriophage peptide, or derivative, comprises or consists of a peptide X⁴SLX⁵DETTKX⁶VLX⁷QAX⁸TDG (SEQ ID NO: 60) or
the second Pf bacteriophage peptide, or derivative, comprises or consists of a peptide AX⁴SLX⁵DETTKX⁶VLX⁷QAX⁸TDG (SEQ ID NO: 61).

44. The immunogenic composition of claim 43, wherein
X⁴ is an aspartic acid or glutamic acid residue;
X⁵ is a leucine or isoleucine residue;
X⁶ is glycine or glutamic acid residue;
X⁷ is threonine or alanine residue; and
X⁸ is glycine or serine residue.

45-46. (canceled)

47. The immunogenic composition of claim 1, wherein the second Pf bacteriophage peptide, or derivative, comprises or consists of a peptide selected from the group consisting of:

| (SEQ ID NO: 20) | |
|---|---|
| | FADSLIDETTKEVLTQAGTDG; |
| (SEQ ID NO: 21) | |
| | FAESLLDETTKGVLAQASTDG; |
| (SEQ ID NO: 22) | |
| | ESLLDETTKGVLAQASTDG; |
| (SEQ ID NO: 23) | |
| | FAESLLDETTKGALAQASTDG; |
| (SEQ ID NO: 24) | |
| | TKEVLTQAGTDG; |
| (SEQ ID NO: 25) | |
| | KEVLTQAGTDG; |
| (SEQ ID NO: 45) | |
| | AESLLDETTKEVLTQAGTDG; |
| (SEQ ID NO: 46) | |
| | ADSLIDETTKEVLTQAGTDG; |
| | and |
| (SEQ ID NO: 47) | |
| | AESLIDETTKGVLAQASTDG. |

48. The immunogenic composition of claim 1, wherein:
each Pf bacteriophage peptide, or derivative thereof, is coupled to the carrier by a linker moiety bonded to a linker-binding moiety, each linker-binding moiety also being bonded to a terminal amino acid of a Pf bacteriophage peptide, or derivative thereof;
each linker moiety is a heterobifunctional linker moiety bonded to a lysine of the carrier;
each linker-binding moiety is a cysteine and the thiol of the cysteine is bonded to the linker moiety and
each carrier is independently a carrier protein selected from keyhole limpet hemocyanin (KLH), cross-reacting material (CRM) of diphtheria toxin, tetanus toxoid, meningococcal outer membrane protein complex, diphtheria toxoid, ExoProteinA (ExoA), or H. influenzae protein D.

49-55. (canceled)

56. The immunogenic composition of claim 51, wherein at least one linker moiety is 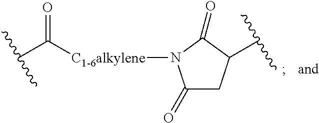
and
the at least one linker moiety bonds to the linker-binding moiety at the 3-position of the succinimide.

57. (canceled)

58. The immunogenic composition of claim 1, further comprising one or more adjuvants.

59. A vaccine comprising the immunogenic composition of claim 1.

60. A method of inducing or enhancing an immune response comprising administering to a subject in need thereof, an effective amount of the immunogenic composition or vaccine of claim 1, with an effective amount of one or more adjuvants; and
wherein the method reduces or prevents Pseudomonas aeruginosa biofilm formation in the subject.

61-66. (canceled)
